(19) United States
(12) Patent Application Publication
(10) Pub. No.: US 2024/0400893 A1
(43) Pub. Date: Dec. 5, 2024

(54) WATER-SOLUBLE TETRAHYDROPYRENE BASED FLUORESCENT POLYMERS

(71) Applicant: Beckman Coulter, Inc., Brea, CA (US)

(72) Inventors: Arunkumar Easwaran, Miami, FL (US); Massimiliano Tomasulo, Miami, FL (US); Sergei Gulnik, San Jose, CA (US)

(21) Appl. No.: 18/696,125

(22) PCT Filed: Sep. 30, 2022
(86) PCT No.: PCT/US2022/077406

§ 371 Date: Mar. 27, 2024

Related U.S. Application Data
(60) Provisional application No. 63/250,986, filed on Sep. 30, 2021.

(51) Int. Cl. C09K 11/06; C09B 69/10; G01N 33/533

(52) U.S. Cl. CPC C09K 11/06; C09B 69/109; G01N 33/533; C09K 2211/1416

(57) ABSTRACT

The present invention provides water-soluble tetrahydropyrene based fluorescent polymers, water-soluble tetrahydropyrene based tandem fluorescent polymers, water-soluble fluorescent polymer complexes, and their use in methods for detecting an analyte in a sample.

This application is being filed on Sep. 30, 2022, as a PCT International Patent application and claims the benefit of and priority to U.S. Provisional Application Ser. No. 63/250,986, filed Sep. 30, 2021, which is incorporated by reference herein in its entirety.

### FIELD OF THE DISCLOSURE

This disclosure relates to water-soluble tetrahydropyrene based fluorescent polymers, water-soluble fluorescent polymer complexes, and their use in methods for detecting analytes in a sample.

### BACKGROUND OF THE DISCLOSURE

Water-soluble fluorescent polymers can be used in a variety of biological applications by generating signals which can be monitored in real time and provide simple and rapid methods for the detection of biological targets and events.

Brightness of a dye is an overall contribution from the extinction coefficient (ε, measure of the amount of light absorbed at a particular wavelength) and fluorescence quantum yield (Φ, measure of the light emitted in the form of radiation from its singlet excited state). Most of the reported organic violet dyes such as coumarin, BODIPY, cyanine, squaraine, etc. are single molecules and show relatively low extinction coefficient in the range of 10,000-70,000 M⁻¹ cm⁻¹ at 405 nm. It has been shown that molecules having multiple chromophores exhibit higher ε value due to the overall contribution from different chromophores. There are various reports on dendrimeric, polymeric backbone and other scaffold approaches where a single molecule contains multiple chromophores.

Many reported polymeric dyes are hydrophobic and were first applied in material applications such as light emitting diodes, solar cells, flat panel displays, etc. Further efforts in polymeric dye technology have targeted improving compatibility with aqueous conditions, dye brightness, and refining spectra properties for additional uses. Only a few reports deal with water-soluble fluorescent polymers for biological applications which are excitable with a 405 nm and 355 nm laser. Therefore, identification of novel water-soluble polymeric cores is needed to expand the availability of water-soluble polymeric dyes for use in biological applications, including for the detection of analytes and use in diagnostic kits etc.

The present disclosure addresses these and other disadvantages of prior art polymers and complexes and methods for detecting analytes in a sample.

### BRIEF SUMMARY OF THE DISCLOSURE

The present disclosure generally provides novel, water-soluble tetrahydropyrene based fluorescent polymers, their complexes, and methods for detecting analytes in a sample using complexes comprising the water-soluble fluorescent polymers conjugated to binding agents.

The disclosure provides a water-soluble fluorescent polymer having the structure of Formula I:

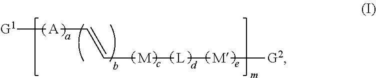

wherein:
A is selected from the group consisting of

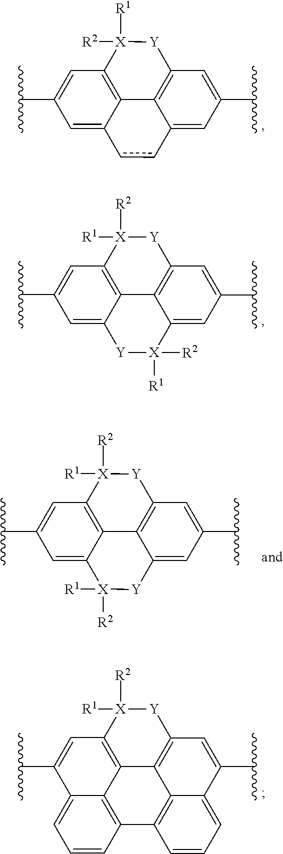

each X is independently selected from the group consisting of a C and Si;
each Y is independently selected from the group consisting of CH₂, CR¹R², and SiR¹R²;
each R¹ is independently selected from the group consisting of a water-solubilizing moiety, alkene, alkyne, cycloalkyl, haloalkyl, (hetero)aryloxy, (hetero)arylamino, PEG, carboxylic acid, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

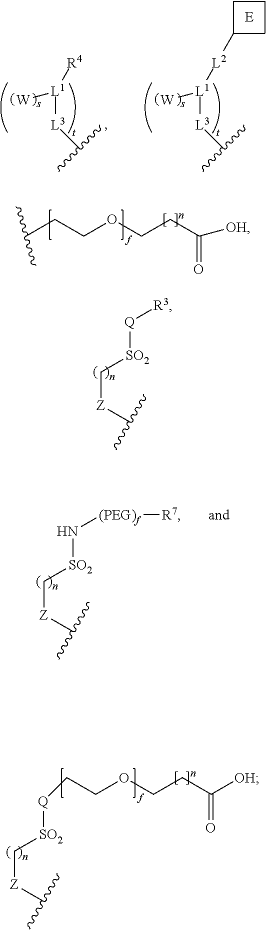

each R² is independently selected from the group consisting of a water-solubilizing moiety, a linker moiety, H, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

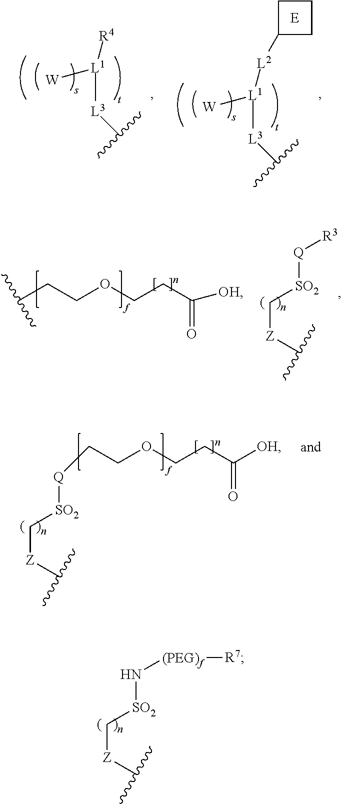

each R³ is independently a water-solubilizing moiety;
Q is a bond, NR⁴, or —CH₂;
Z is CH₂, O, or NR⁴;
W is a water solubilizing moiety;
R⁴ is independently selected the group consisting of H, a PEG group, a water-solubilizing moiety, a linker moiety, a chromophore, a linked chromophore, a functional group, a linked functional group, a functional tag, a linked functional tag, a substrate, a linked substrate, a binding agent, a linked binding agent, L²-E, halogen, hydroxyl, alkylamino, alkyl, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂—Q—R³, a C₂-C₁₈ (hetero)aryl group, amide, amine, carbamate, carboxylic acid, carboxylate ester, maleimide, activated ester, N-hydroxysuccinimidyl, hydrazine, hydrazide, hydrazone, azide, alkyne, aldehyde, and thiol, or protected groups thereof, optionally conjugated to a chromophore, substrate, functional tag, or binding agent;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
at least one of R¹, R², R³, or R⁴ comprises a water-solubilizing moiety;
each optional M and M′ is independently a polymer modifying unit evenly or randomly distributed along the polymer main chain and is optionally substituted with one or more optionally substituted R¹, R², R³, or R⁴ groups;
each E is independently a chromophore, acceptor dye, substrate, functional tag, or binding agent;
each optional L is a linker, optionally conjugated to a chromophore, acceptor dye, substrate, functional tag, or binding agent;
L¹, L², and L³ are each independently selected linker moieties;
G¹ and G² are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, optionally conjugated to a chromophore, acceptor dye, substrate, functional tag, or binding agent;
a, c, d, and e define the mol % of each unit within the structure which each can be evenly or randomly repeated and where each a is a mol % from 10 to 100%, each c is a mol % from 0 to 90%, each d is a mol % from 0 to 25%, and each e is a mol % from 0 to 90%;
each b is independently 0 or 1;
each f is independently an integer from 0 to 50;
m is an integer from 1 to about 10,000;
each n is independently an integer from 1 to 20;
each s is independently 1 or 2; and
each t is independently 1, 2, or 3.

In some cases, the water-soluble fluorescent polymer of the invention has a structure selected from the group consisting of Formulas (Ia)-(Id):

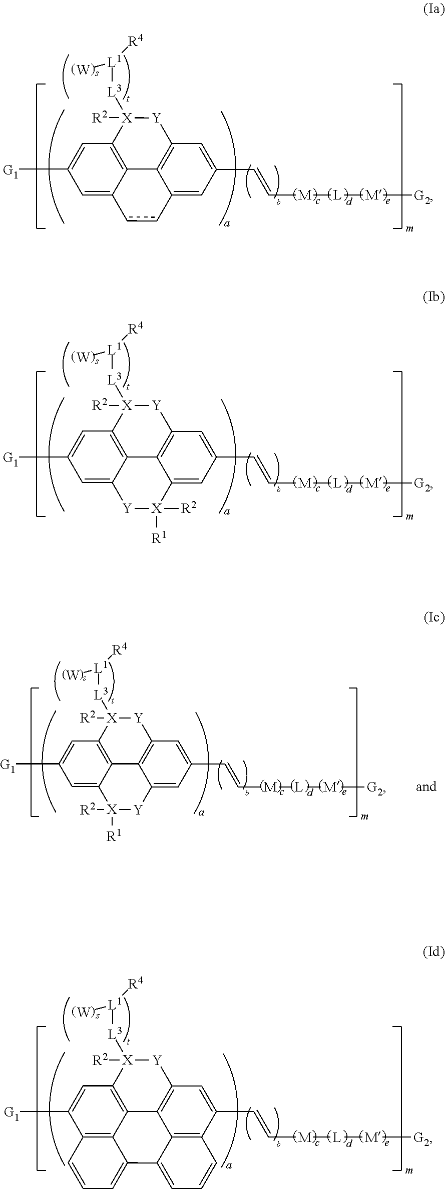

wherein
each of X, Y, R¹, R², R⁴, M, M′, L, L¹, L³, W¹, G¹, G², a, b, c, d, e, m, s, and t are as described above.

In some cases, the water-soluble fluorescent polymer of the invention has a structure selected from the group consisting of Formulas (IIa)-(IId):

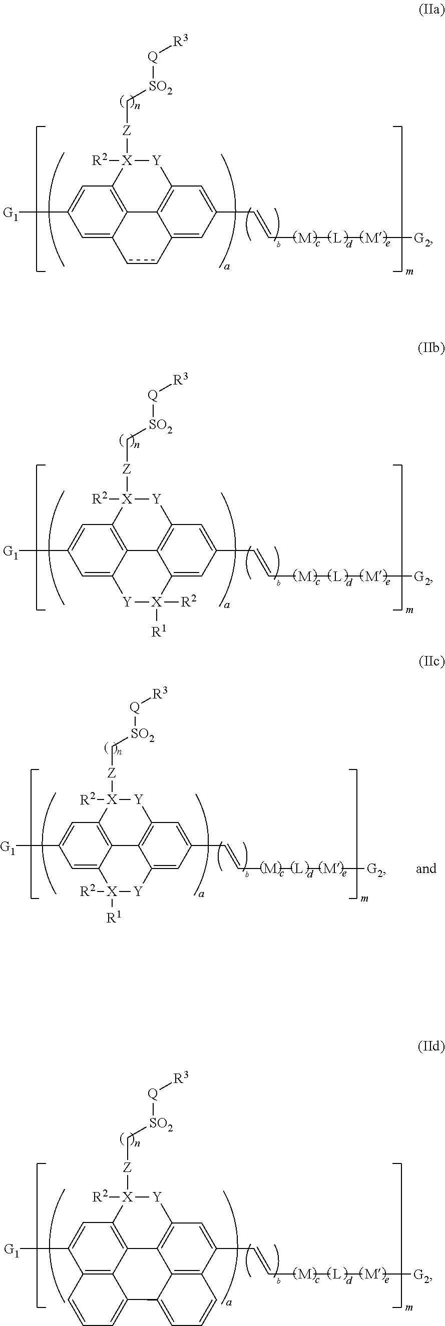

wherein
each of X, Y, R¹, R², R³, Q, Z, R⁴, M, M′, R⁵, L, G¹, G², a, b, c, d, e, m, and n are as described above.

In some cases, the water-soluble fluorescent polymer of the invention has a structure selected from the group consisting of Formulas (IIIa)-(IIId):

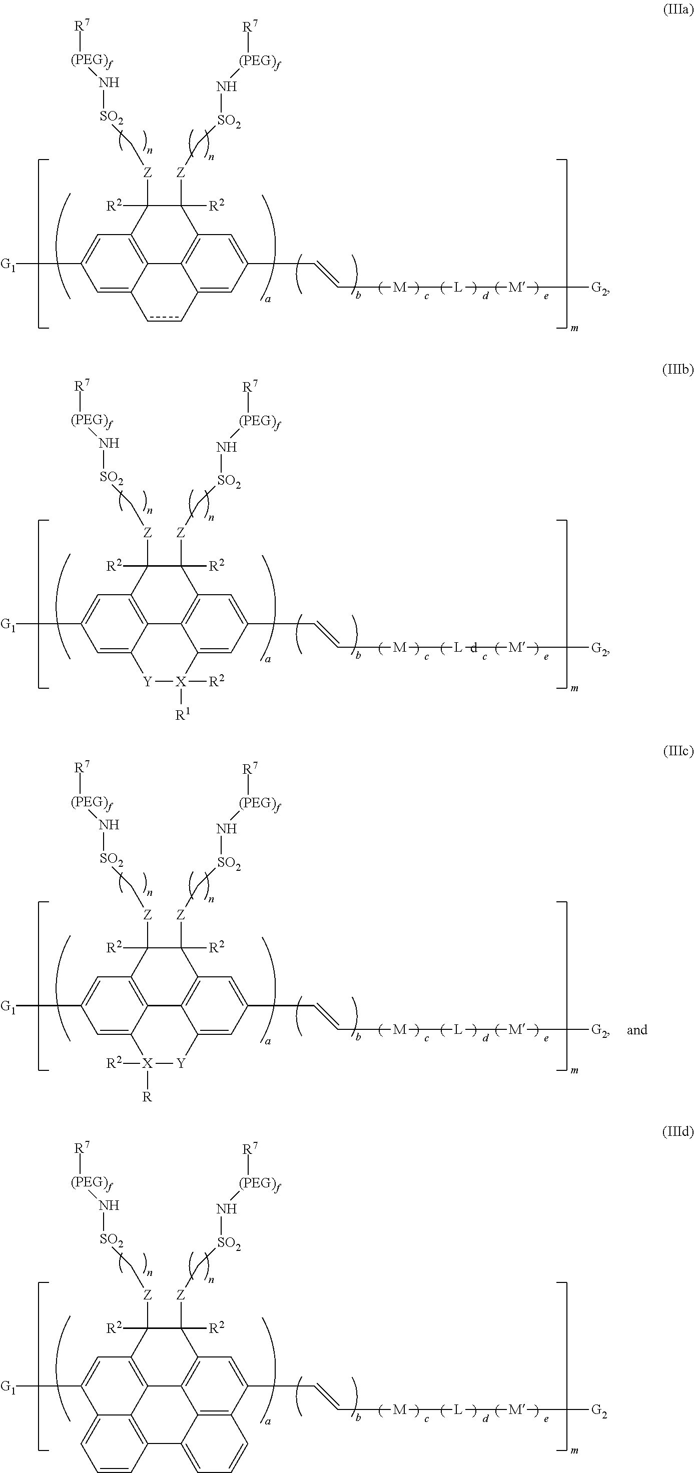

wherein,
each of X, Y, R¹, R², R³, Q, Z, R⁴, M, R⁵, R⁷, L, G¹, G², a, b, c, d, e, f, m, and n are as described above.

In some cases, the water-soluble fluorescent polymer of the invention a structure selected from the group consisting of Formulas (IVa)-(IVd):

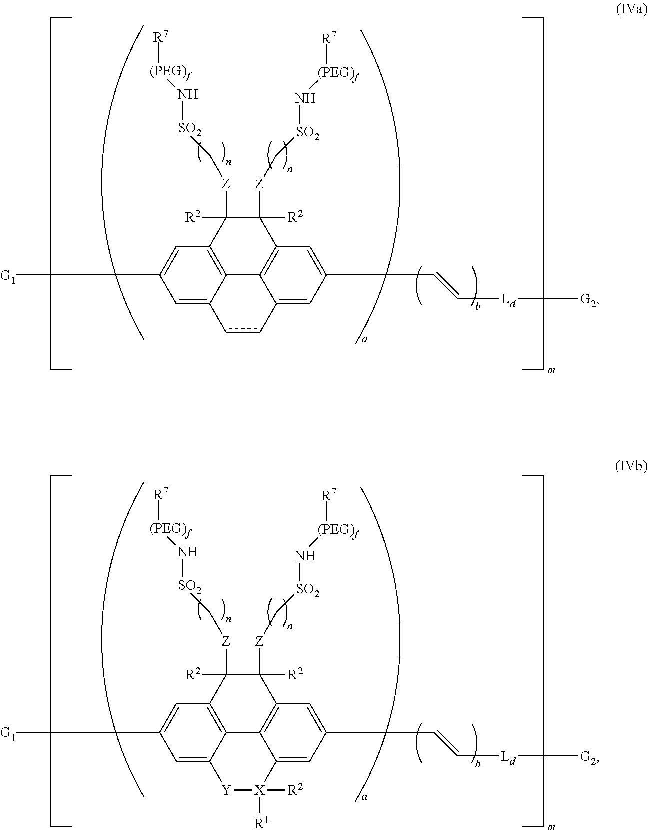

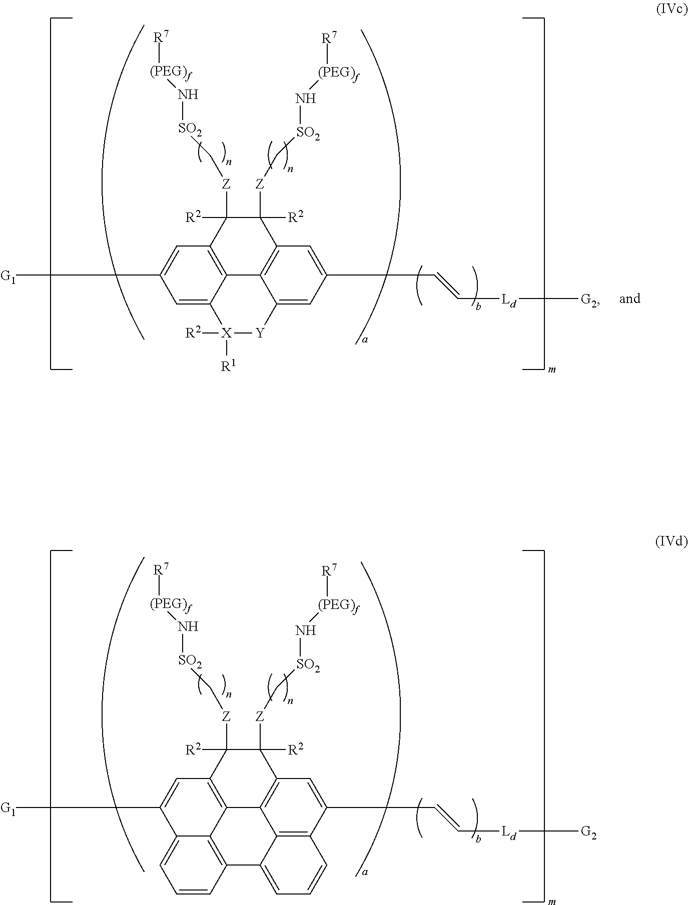

wherein,
each of X, Y, R¹, R², R³, Q, Z, R⁴, R⁷, L, G¹, G², a, b, d, f, m, and n are as described above.

In some cases, the water-soluble fluorescent polymer of the invention has the structure selected from the group consisting of Formulas (Va)-(Vd):

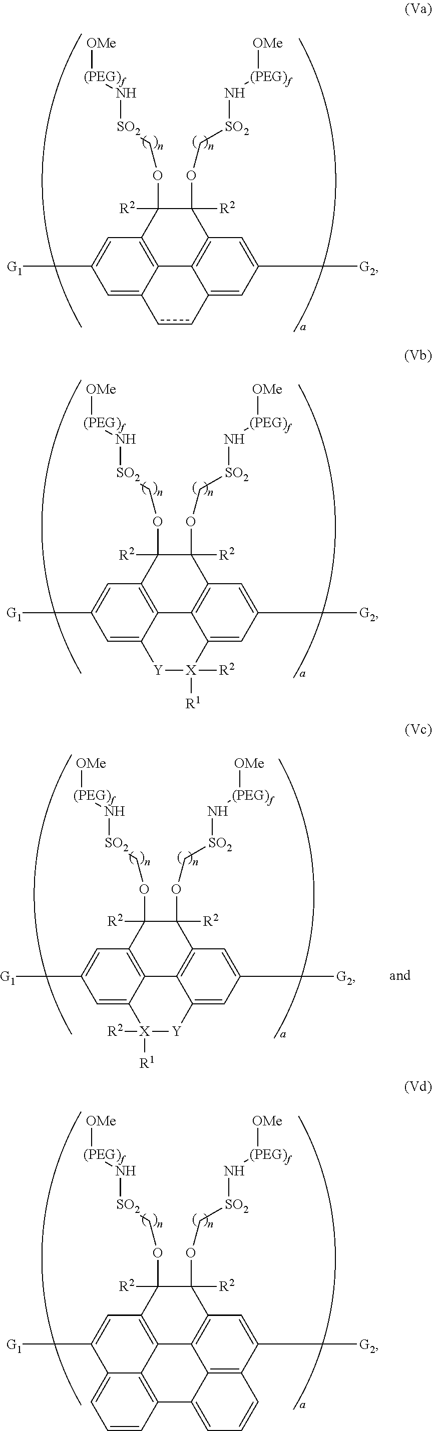

wherein
each of X, Y, R¹, R², R³, Q, Z, R⁴, G¹, G², a, f, and n are as described above.

In some cases, the water-soluble fluorescent polymer of the invention has the structure selected from the group consisting of Formula (VIa) and (VIb):

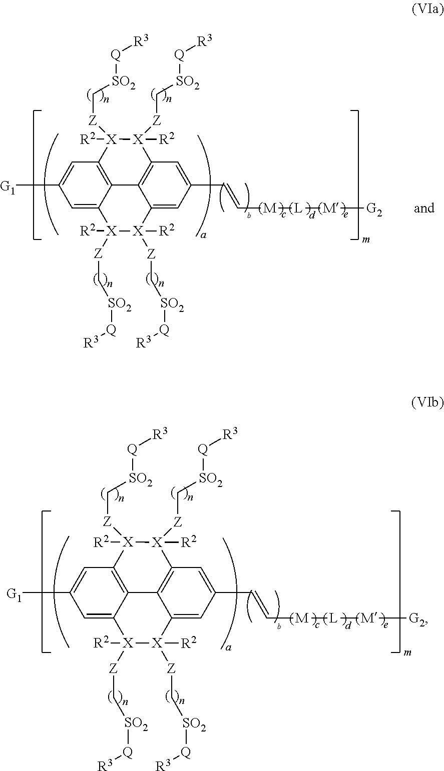

wherein
each of X, R¹, R², R³, Q, Z, R⁴, M, M′, R⁵, L, G¹, G², a, b, c, d, e, m, and n are as described above.

In some cases, the water-soluble fluorescent polymer of the invention has the structure selected from the group consisting of Formula (VIIa) and (VIIb),

wherein:

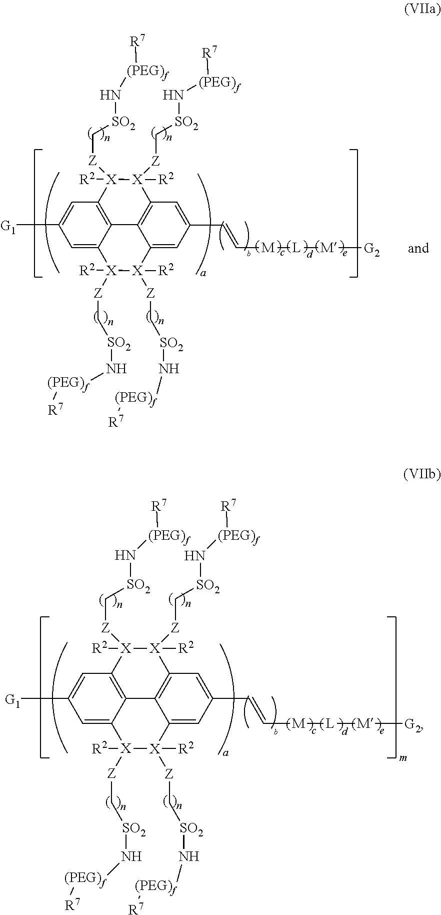

wherein
each of X, R¹, R², R³, M, M′, Q, Z, R⁴, R⁷, L, G¹, G², a, b, d, e, f, m, and n are as described above.

In some cases, the water-soluble fluorescent polymer of the invention has the structure selected from the group consisting of Formula (VIIIa) and (VIIIb):

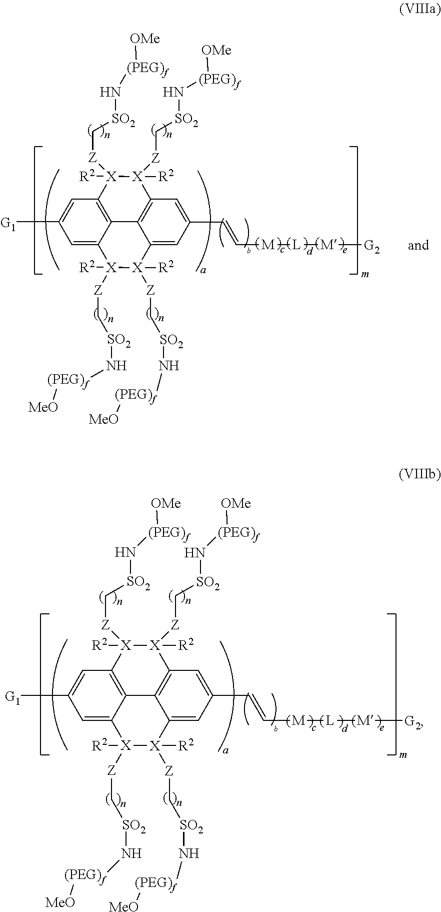

wherein
each of X, R¹, R², R³, M, M′, Q, Z, R⁴, R⁷, G¹, G², a, b, c, d, e, f, and n are as described above.

In some cases, the water-soluble fluorescent polymer of the invention is a water-soluble fluorescent copolymer and has the structure of Formula (IX):

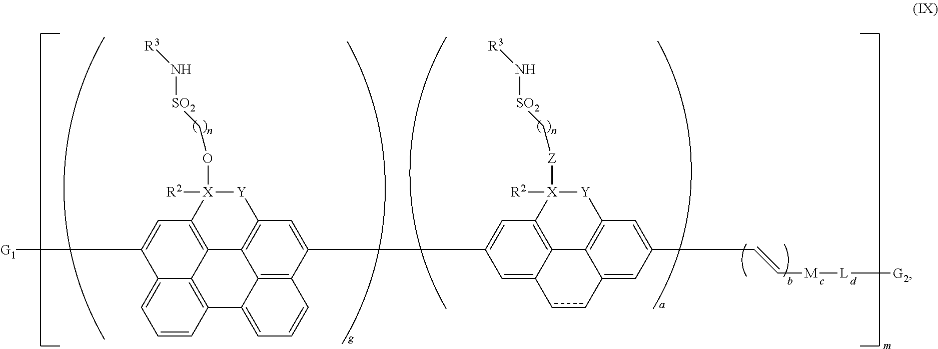

wherein
each of X, Y, R¹, R², R³, Z, R⁴, M, R⁵, L, G¹, G², a, b, c, d, m, and n are as described above.

In some cases, the water-soluble fluorescent polymer of the invention is a water-soluble fluorescent copolymer and has the structure of Formula (X).

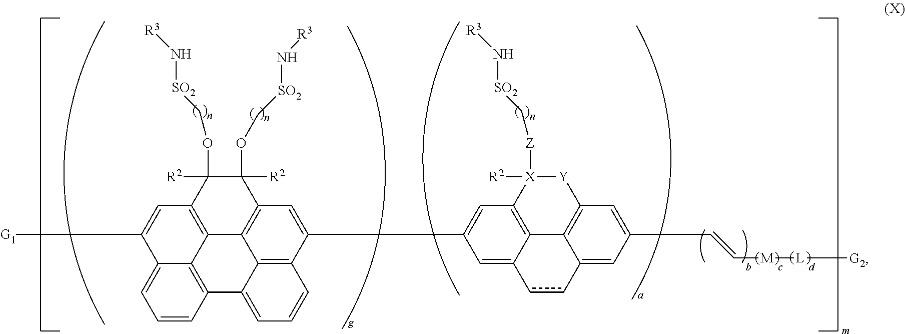

wherein
each of X, Y, R¹, R², R³, Q, Z, R⁴, M, R⁵, L, G¹, G², a, b, c, d, m, and n are as described above.

In some cases, the water-soluble fluorescent polymer of the invention is a water-soluble fluorescent copolymer and has the structure of Formula (XI):

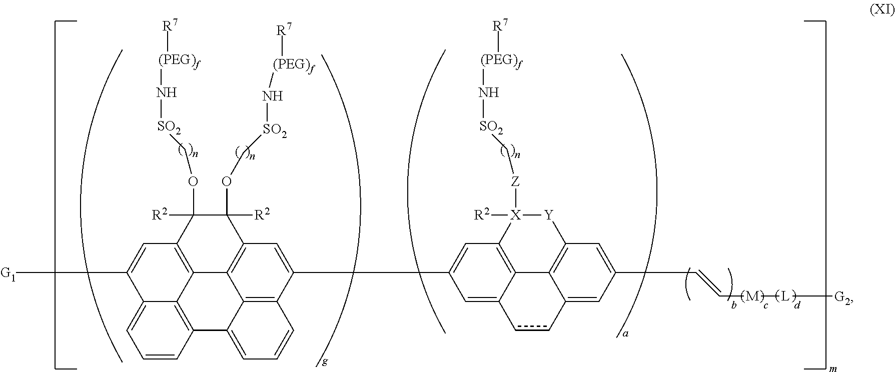

wherein
each of X, Y, R¹, R², R³, Q, Z, R⁴, M, R⁵, R⁷, L, G¹, G², a, b, c, d, f, m, and n are as described above.

In some cases, the water-soluble fluorescent polymer of the invention is a water-soluble fluorescent copolymer and has the structure of Formula (XII):

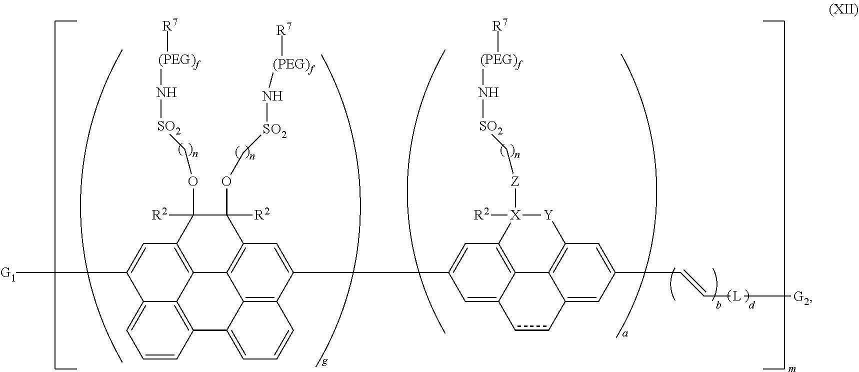

wherein
each of X, Y, R¹, R², R³, Q, Z, R⁴, M, R⁵, R⁷, L, G¹, G², a, b, c, d, f, m, and n are as described above.

In some cases, the water-soluble fluorescent polymer of the invention is a water-soluble fluorescent copolymer and has the structure of Formula (XIII):

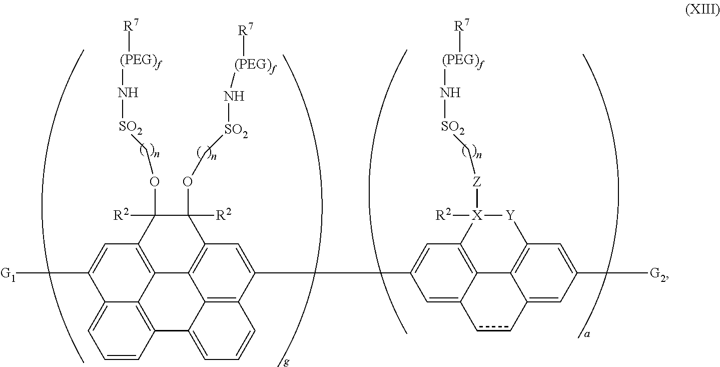

wherein
each of X, Y, R¹, R², R³, Q, Z, R⁴, M, R⁵, L, G¹, G², a, b, c, d, f, m, and n are as described above.

In some cases, the water-soluble fluorescent polymer of the invention is a water-soluble fluorescent copolymer and has the structure of Formula (XIV):

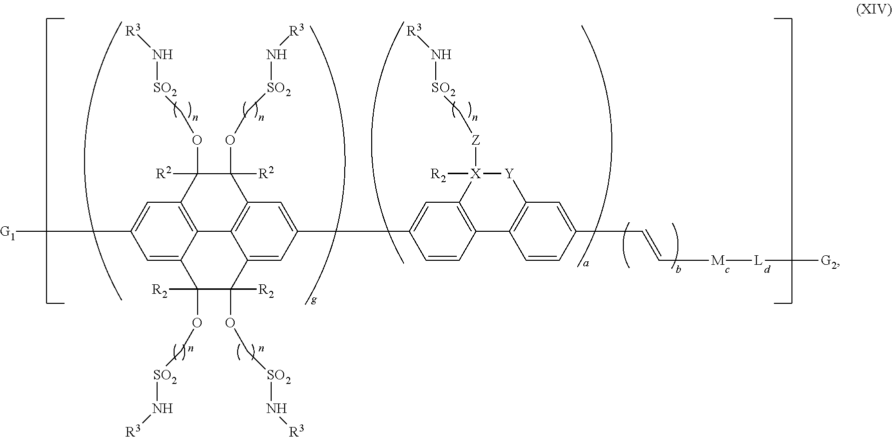

wherein
each of X, Y, R¹, R², R³, Q, Z, R⁴, M, R⁵, L, G^{L}, G², a, b, c, d, m, and n are as described above.

In some cases, the water-soluble fluorescent polymer of the invention is a water-soluble fluorescent copolymer and has the structure of Formula (XV):

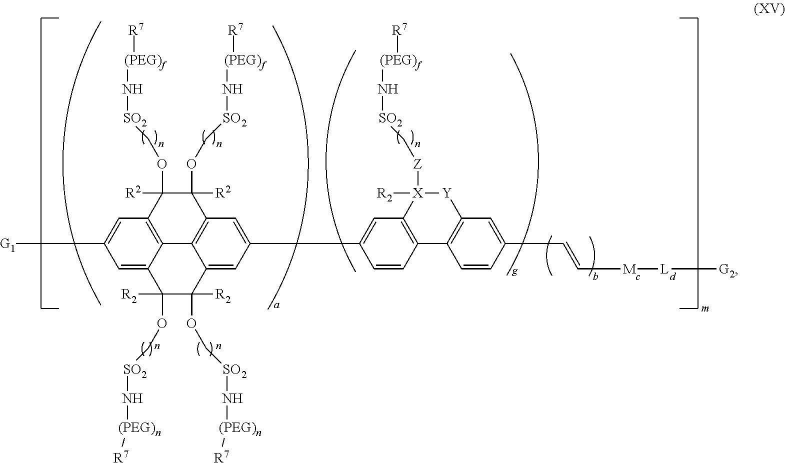

wherein
each of X, Y, R¹, R², R³, Q, Z, R⁴, M, R⁵, R⁷, L, G¹, G², a, b, c, d, f, m, and n are as described above.

In some cases, the water-soluble fluorescent polymer of the invention is a water-soluble fluorescent copolymer and has the structure of Formula (XVI):

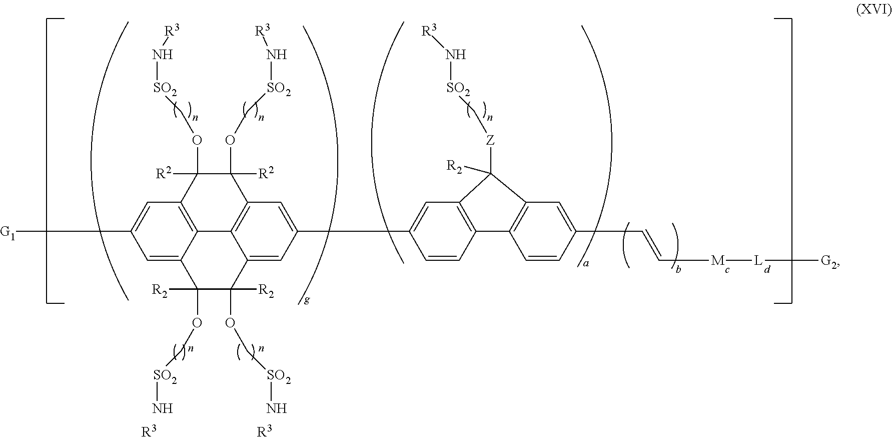

wherein
each of X, R¹, R², R³, Q, Z, R⁴, M, R⁵, L, G¹, G², a, b, c, d, m, and n are as described above.

In some cases, the water-soluble fluorescent polymer of the invention is a water-soluble fluorescent copolymer and has a structure of Formula (XVII):

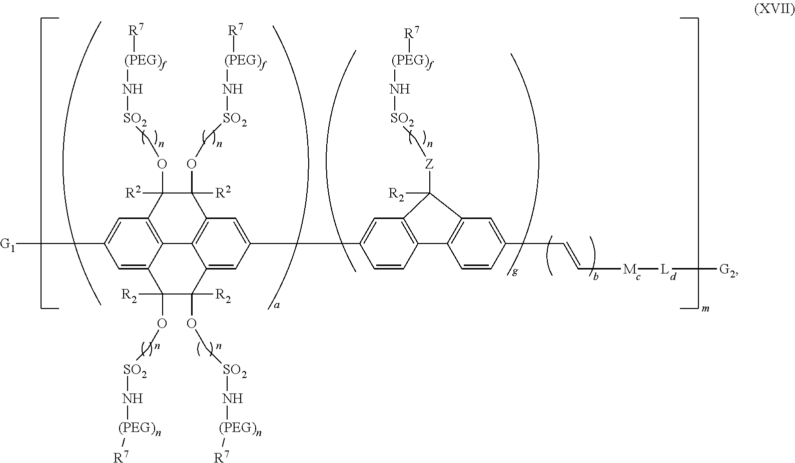

wherein
each of X, R¹, R², R³, Q, Z, R⁴, M, R⁵, R⁷, L, G¹, G², a, b, c, d, f, m, and n are as described above.

In some cases, each “L” is independently selected from the group consisting of

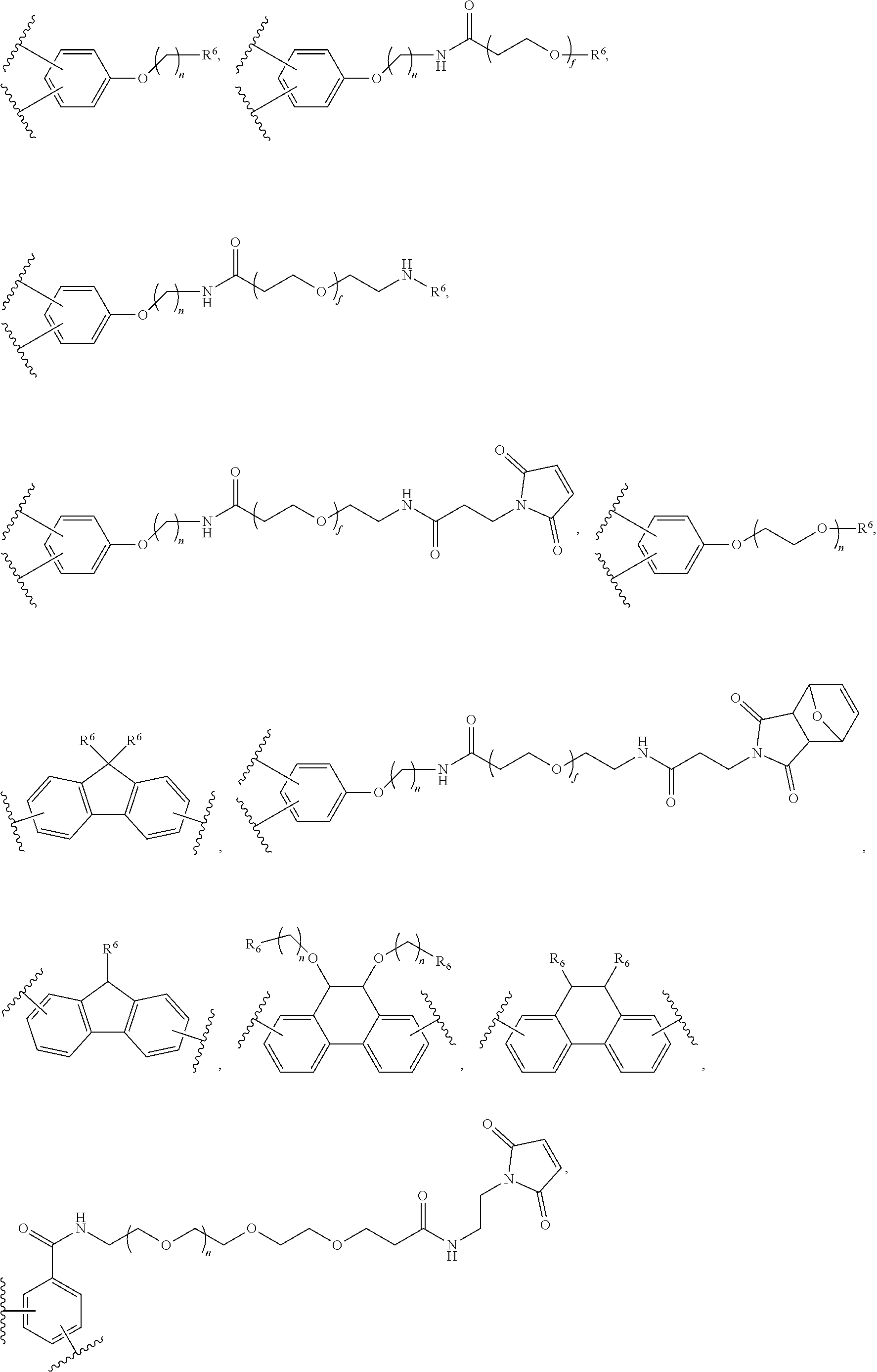

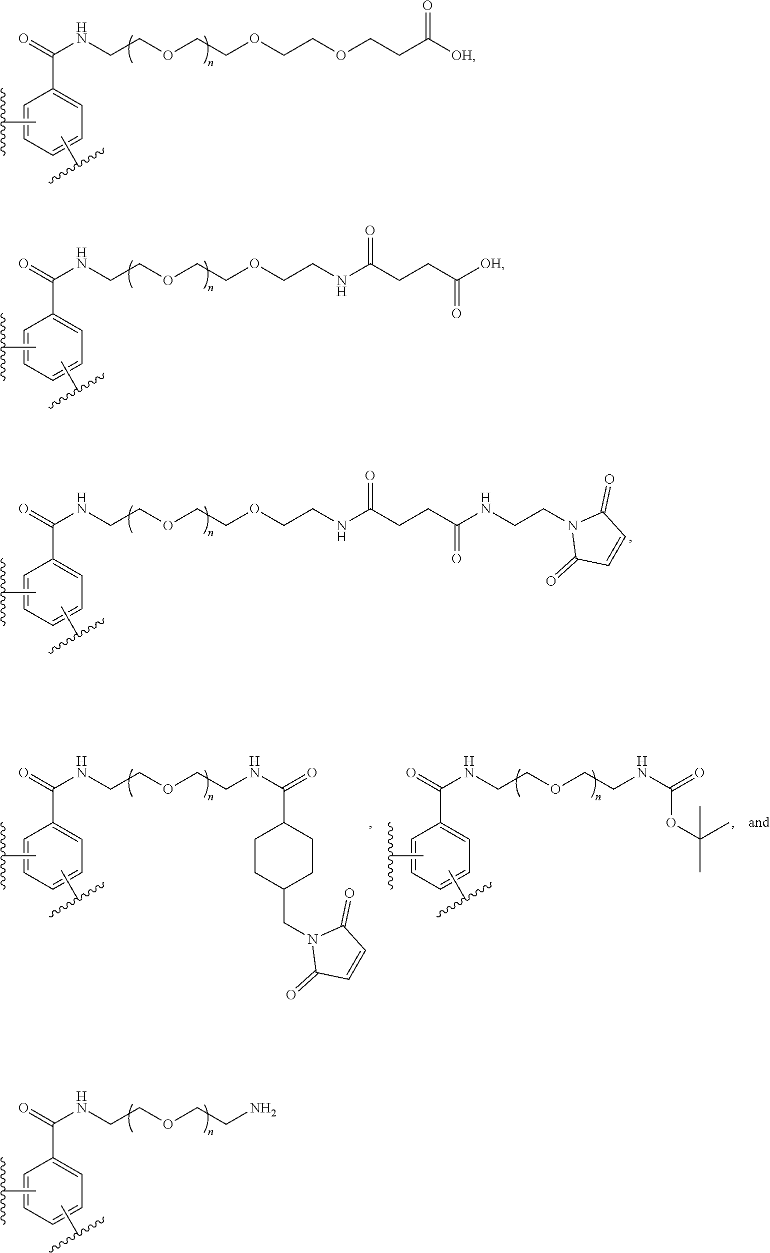

wherein,
each R⁶ is independently selected from the group consisting of H, OH, SH, NHCOO-t-butyl, (CH₂)ₙCOOH, (CH₂)ₙCOOCH₃, (CH₂)ₙCOOR¹⁵, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}OR¹⁵, (CH₂)ₙNHCO(CH₂CH₂O)_{f}CH₃, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OR¹⁵, (CH₂)ₙ(OCH₂CH₂)_{f}OCH₃, N-maleimide, halogen, azide, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino, benzyl optionally substituted with one or more halogen, hydroxyl, C₁-C₁₂ alkoxy, or (OCH₂CH₂)_{f}OCH₃,

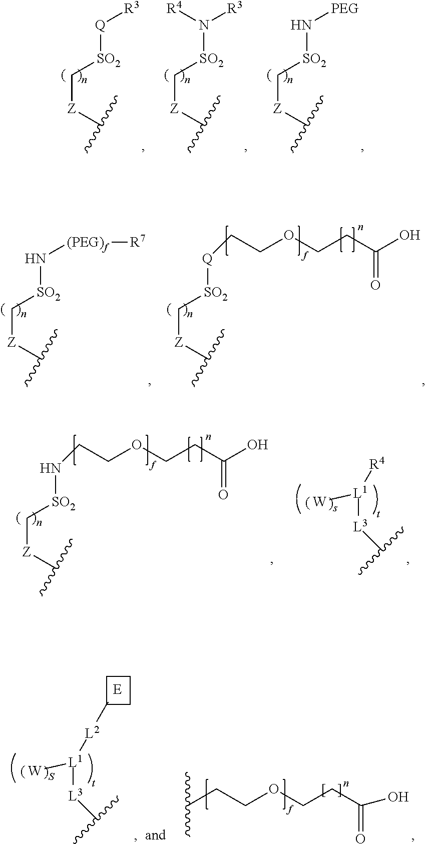

wherein
each of R³, R⁴, R⁷, L¹, L², L³, E, Z, Q, f, n, s, and t are each as described above.

In some embodiments, the present invention provides a method for detecting an analyte in a sample comprising providing a sample that is suspected of containing the analyte; and contacting the sample with a binding agent conjugated to a water-soluble fluorescent polymer having the structure of any of Formulas I-XVII as disclosed above, wherein the binding agent is capable of interacting with the analyte. In some embodiments, the method further comprises, applying a light source to the sample that can excite the polymer; and detecting whether light is emitted from the conjugated water-soluble fluorescent polymer complex (i.e., binding agent conjugated to the water-soluble fluorescent polymer).

In some embodiments, the binding agent is an antibody, antibody fragment, protein, peptide, affinity ligand, antibody, antibody fragment, carbohydrate, lipid, nucleic acid or an aptamer. In some embodiments, the binding agent is an antibody.

In some embodiments, the method is configured for flow cytometry. In some embodiments, the conjugated water-soluble fluorescent polymer complex consists essentially of a water-soluble fluorescent polymer and a binding agent. In some embodiments, the conjugated water-soluble fluorescent polymer complex consists essentially of a water-soluble fluorescent polymer and an antibody. In some embodiments, the binding agent is bound to a substrate. In some embodiments, the water-soluble fluorescent polymer is bound to a substrate. In some embodiments, the analyte is a protein expressed on a cell surface.

In some embodiments, the method is configured for cell sorting. In some embodiments, the binding agent is bound to a substrate. In some embodiments, the analyte is a protein expressed on a cell surface.

In some embodiments, the method is configured as an immunoassay. In some embodiments, the method further comprises providing additional binding agents for detecting additional analytes simultaneously.

In some embodiments, the present invention provides for a kit comprising any water-soluble fluorescent polymer having the structure of any of Formulas I-XVII as disclosed above.

### BRIEF DESCRIPTION OF THE DRAWINGS

FIG. 1 shows a representative procedure for tandem dye formation and antibody conjugation from carboxylic water-soluble fluorescent polymer.

FIG. 2 shows representative physical and chemical characteristics of the water-soluble fluorescent polymer complexes of the invention including absorption, fluorescence, brightness, molecular weight, polydispersity, and dye to protein ratio. Representative ranges of parameters in some instances are shown in the table.

### DETAILED DESCRIPTION OF THE DISCLOSURE

### I. General

The present disclosure provides novel, water-soluble fluorescent polymers and methods for detecting analytes in a sample using conjugated water-soluble fluorescent polymer complexes comprising the water-soluble fluorescent polymers conjugated to binding agents. The water-soluble fluorescent polymers of the present disclosure demonstrate significantly increased brightness compared to other dyes.

### II. Definitions

The abbreviations used herein have their conventional meaning within the chemical and biological arts.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “functional tag” refers to substituent that can be recognized by antibodies or other binding agents. The functional tag can be used, for example, with colorimetric reagents, chemiluminescent reagents, or the like for convenient identification, isolation, and/or quantification. The functional tag can be a biotin, a digoxigenin, a peptide tag such as a FLAG peptide, an oligonucleotide, or a polynucleotide. As used herein, the term “FLAG peptide” refers to an oligopeptide or a polypeptide containing the amino acid sequence Asp-Tyr-Lys-Asp-Asp-Asp-Asp-Lys (i.e., DYKDDDDK). FLAG peptides and variants thereof are described for example, in U.S. Pat. No. 4,703,004 to Hopp, et al., which patent is incorporated herein by reference. Other peptides that can be used in place of a FLAG peptide include, but are not limited to, HA peptide tags containing the sequence Tyr-Pro-Tyr-Asp-Val-Pro-Asp-Tyr-Ala (i.e., YPYDVPDYA), His₆ peptide tags containing the sequence His-His-His-His-His-His (i.e., HHHHHH), and Myc peptide tags containing the sequence Glu-Gln-Lys-Leu-Ile-Ser-Glu-Glu-Asp-Leu (i.e., EQKLISEEDL). The peptide tags can be recognized by antibodies or other binding agents for use with colorimetric reagents, chemiluminescent reagents, or the like for convenient identification and/or quantification. Nucleotides (e.g., RNA, single-stranded DNA, or double-stranded DNA) can be recognized by a complementary primer or other complementary nucleotide as described, for example, in WO 2016/019929 (Navratil, et al.), which publication is incorporated herein by reference. As used herein, the term “digoxigenin” refers to 3-[(3S,5R,8R,9S,10S,12R,13S,14S,17R)-3,12,14-trihydroxy-10,13-dimethyl-1,2,3,4,5,6,7,8,9,11, 12,15,16,17-tetradecahydrocyclopenta[a]-phenanthren-17-yl]-2H-furan-5-one (CAS Registry No. 1672-46-4) and substituted analogs thereof. As used herein, the term “biotin” refers to 5-[(3aS,4S,6aR)-2-oxohexahydro-1H-thieno[3,4-d]imidazol-4-yl]pentanoic acid (CAS Registry No. 58-85-5) and substituted analogs thereof.

As used herein, the term “ammonium” by itself or as part of another substituent refers to a cation having the formula NHR₃⁺ where each R group, independently, is hydrogen or a substituted or unsubstituted alkyl, aryl, aralkyl, or alkoxy group. Preferably, each of the R groups is hydrogen.

As used herein, the term “oligoether” is understood to mean an oligomer containing structural repeat units having an ether functionality. As used herein, an “oligomer” is understood to mean a molecule that contains one or more identifiable structural repeat units of the same or different formula.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein some or all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

As used herein, the term “alkyl” by itself or as part of another substituent refers to a straight or branched, saturated, aliphatic radical having the number of carbon atoms indicated. Alkyl groups can be optionally substituted alkyl groups. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together. As used herein, the term “lower alkyl” by itself or as part of another substituent refers to an C₁-C₆ alkyl including, but is not limited to, methyl, ethyl, propyl, isopropyl, butyl, isobutyl, sec-butyl, tert-butyl, pentyl, isopentyl, hexyl, etc.

As used herein, the term “cycloalkyl” by itself or as part of another substituent refers to a saturated or partially unsaturated, monocyclic, fused bicyclic or bridged polycyclic ring assembly containing from 3 to 12 ring atoms, or the number of atoms indicated monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane.

As used herein, the term “haloalkyl” by itself or as part of another substituent refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “halogen” by itself or as part of another substituent refers to fluorine, chlorine, bromine and iodine.

As used herein, the term “alkoxy” by itself or as part of another substituent refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

As used herein, the term “alkene” or “alkenyl” by itself or as part of another substituent refers to either a straight chain, branched chain, or cyclic hydrocarbon, having at least one double bond between two carbon atoms. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkene group is typically monovalent, but can be divalent, such as when the alkenyl group links two moieties together.

As used herein, the term “alkyne” or “alkynyl” by itself or as part of another substituent refers to either a straight chain or branched hydrocarbon, having at least one triple bond between two carbon atoms. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acyl group can optionally also include heteroatoms within the meaning herein. Examples of acyl groups include, but are not limited to, a nicotinoyl group (pyridyl-3-carbonyl) acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

As used herein, the term “aryl” by itself or as part of another substituent refers to cyclic aromatic hydrocarbon groups that do not contain heteroatoms in the aromatic ring assembly. “Aryl” groups can be a monocyclic or fused bicyclic, tricyclic or greater, aromatic ring assembly containing 6 to 16 ring carbon atoms. For example, aryl may be, but is not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred as aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

As used herein, the term “aryloxy” by itself or as part of another substituent refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “(hetero)aryloxy” as used herein means an —O-heteroaryl group, wherein heteroaryl is as defined below. The term “(hetero)aryloxy” is used to indicate the moiety is either an aryloxy or (hetero)aryloxy group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

As used herein, the term “heteroaryl” by itself or as part of another substituent refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

In some embodiments, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′—and —S(O)₂NR′—is selected from hydrogen or unsubstituted (C₁-C₆)alkyl.

As used herein, the terms “polyethylene glycol” or “PEG” refer to the family of biocompatible water-solubilizing linear polymers based on the ethylene glycol monomer unit described by the formula —(CH₂—CH₂—O—)ₙ— or a derivative thereof. In some embodiments, “n” is 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, carboxylic acid, carboxylate ester, acyloxy, and amido terminal and/or substituent groups.

As used herein, the term “amine” by itself or as part of another substituent as used herein refers to an alkyl groups as defined within, having one or more amino groups. The amino groups can be primary, secondary or tertiary. The alkyl amine can be further substituted with a hydroxy group. Amines useful in the present disclosure include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present disclosure.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “amide” refers to a functional group having a carbonyl group attached to an amine group, having the general formula RC(═O)NR′R″, where R, R′, and R″ represent organic groups or hydrogen atoms. The term “amido” refers to a substituent containing an amide group.

As used herein, the term “(hetero)arylamino” by itself or as part of another substituent refers an amine radical substituted with an aryl group (e.g., —NH-aryl). An arylamino may also be an aryl radical substituted with an amine group (e.g., -aryl-NH₂). Arylaminos may be substituted or unsubstituted.

As used herein, the term “carbamate” by itself or as part of another substituent refers to the functional group having the structure —NR″CO₂R′, where R′ and R″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

As used herein, the term “carboxylic acid” by itself or as part of another substituent refers to a structure R—COOH where R is a carbon-containing group of atoms.

As used herein, the term “carboxylate” by itself or as part of another substituent refers to the conjugate base of a carboxylic acid, which generally can be represented by the formula RCOO⁻. For example, the term “magnesium carboxylate” refers to the magnesium salt of the carboxylic acid The term “carboxylate ester” as used herein by itself or as part of another substituent refers to a compound derived from a carboxylic acid, which generally can be represented by the formula RCOOR′ where R′ can be an alkyl, alkene, alkyne, haloalkyl, heteroalkyl, cycloalkyl, aryl, heteroaryl, (unsubstituted aryl)alkyl, and (unsubstituted aryl)oxy-alkyl or other carbon-containing group of atoms. R′ can optionally contain functional groups.

As used herein, the term “sulfonate functional group” or “sulfonate” either by itself or as part of another substituent refers to both the free sulfonate anion (—S(═O)₂O—) and salts thereof. Therefore, the term sulfonate encompasses sulfonate salts such as sodium, lithium, potassium and ammonium sulfonate.

As used herein, the term “sulfonamido” by itself or as part of another substituent refers to a group of formula —SO₂NR— where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “sulfonamide” by itself or as part of another substituent refers to a group of formula —SO₂NR₂ where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “sulfinamide” by itself or as part of another substituent refers to a group of formula —SONR₂— where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “activated ester” or “active esters” by itself or as part of another substituent refers to carboxyl-activating groups employed in peptide chemistry to promote facile condensation of a carboxyl group with a free amino group of an amino acid derivative. Descriptions of these carboxyl-activating groups are found in general textbooks of peptide chemistry, for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

As used herein, the terms “hydrazine” and “hydrazide” by themselves or as part of another substituent refer to compounds that contain singly bonded nitrogens, one of which is a primary amine functional group.

As used herein, the term “aldehyde” by itself or as part of another substituent refers to a chemical compound that has a —CHO group.

As used herein, the term “thiol” by itself or as part of another substituent refers to a compound that contains the functional group composed of a sulfur-hydrogen bond. The general chemical structure of the thiol functional group is R—SH, where R represents an alkyl, alkene, aryl, or other carbon-containing group of atoms.

As used herein, the term “silyl” by itself or as part of another substituent refers to Si(R^{z})₃ wherein each R^{z} independently is alkyl, aryl or other carbon-containing group of atoms.

As used herein, the term “diazonium salt” by itself or as part of another substituent refers to a group of organic compounds with a structure of R—N₂⁺X⁻, wherein R can be any organic group (e.g., alkyl or aryl) and X is an inorganic or organic anion (e.g., halogen).

As used herein, the term “triflate” by itself or as part of another substituent also referred to as trifluoromethanesulfonate, is a group with the formula CF₃SO₃.

As used herein, the term “boronic acid” by itself or as part of another substituent refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. In some embodiments, the boronic ester moiety is a 5-membered ring. In some other embodiments, the boronic ester moiety is a 6-membered ring. In some other embodiments, the boronic ester moiety is a mixture of a 5-membered ring and a 6-membered ring.

As used herein, the term “maleimide” by itself or as part of another substituent refers a structure

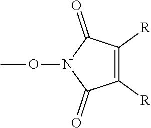

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “hydrazone” by itself or as part of another substituent refers to a structure

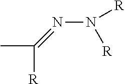

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “azide” by itself or as part of another substituent refers to a structure-N₃

As used herein, the term “N-hydroxysuccinimidyl” by itself or as part of another substituent refers to a structure

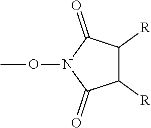

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphoramide” by itself or as part of another substituent refers to a structure

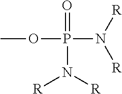

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphonamidate” by itself or as part of another substituent refers to a structure

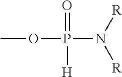

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphinamide” by itself or as part of another substituent refers to a structure

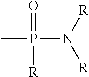

where R can be, for example, a water solubilizing moiety, hydrogen, alkyl, alkene, alkyne, cycloalkyl, haloalkyl, aryl, or other and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

The term “chromophore” refers to a part (atom or group of atoms) of a compound in which the electronic transition responsible for a given spectral band is approximately localized. The chromophore may be any suitable chromophore, fluorophore, lumiphore, or acceptor dye. The chromophore may have a functional group (e.g., a carboxylate moiety, an amino moiety, a haloalkyl moiety, or the like) that can be covalently bonded. Examples of suitable chromophores include, but are not limited to, those described in U.S. Pat. Nos. 7,687,282; 7,671,214; 7,446,202; 6,972,326; 6,716,979; 6,579,718; 6,562,632; 6,399,392; 6,316,267; 6,162,931; 6,130,101; 6,005,113; 6,004,536; 5,863,753; 5,846,737; 5,798,276; 5,723,218; 5,696,157; 5,658,751; 5,656,449; 5,582,977; 5,576,424; 5,573,909; and 5,187,288, which patents are incorporated herein by reference in their entirety.

Typical fluorophores may include, for example, fluorescent dyes, semiconductor nanocrystals, lanthanide chelates, polynucleotide-specific dyes, and green fluorescent protein.

Exemplary fluorescent dyes may include, for example, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2® Cy3®, Cy3.5®. Cy5®, Cy5.5®, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br.sub.2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, conjugates thereof, combinations thereof, and the like. Exemplary lanthanide chelates may include europium chelates, terbium chelates, and samarium chelates, and the like.

A wide variety of fluorescent semiconductor nanocrystals (“SCNCs”) are known in the art; methods of producing and utilizing semiconductor nanocrystals are described in: PCT Publ. No. WO 99/26299 published May 27, 1999, inventors Bawendi et al.; U.S. Pat. No. 5,990,479 issued Nov. 23, 1999 to Weiss et al.; and Bruchez et al., Science 281:2013, 1998. Semiconductor nanocrystals can be obtained with very narrow emission bands with well-defined peak emission wavelengths, allowing for a large number of different SCNCs to be used as signaling chromophores in the same assay, optionally in combination with other non-SCNC types of signaling chromophores.

Exemplary polynucleotide-specific dyes may include acridine orange, acridine homodimer, actinomycin D, 7-aminoactinomycin D (7-AAD), 9-amino-6-chloro-2-methoxyacridine (ACMA), BOBO™-1 iodide (462/481), BOBO™-3 iodide (570/602), BO-PRO™-1 iodide (462/481), BO-PRO™-3 iodide (575/599), 4′,6-diamidino-2-phenylindole, dihydrochloride (DAPI), 4′,6-diamidino-2-phenylindole, dihydrochloride (DAPI), 4′,6-diamidino-2-phenylindole, dilactate (DAPI, dilactate), dihydroethidium (hydroethidine), dihydroethidium (hydroethidine), dihydroethidium (hydroethidine), ethidium bromide, ethidium diazide chloride, ethidium homodimer-1 (EthD-1), ethidium homodimer-2 (EthD-2), ethidium monoazide bromide (EMA), hexidium iodide, Hoechst 33258, Hoechst 33342, Hoechst 34580, Hoechst S769121, hydroxystilbamidine, methanesulfonate, JOJO™-1 iodide (529/545), JO-PRO™-1 iodide (530/546), LOLO™-1 iodide (565/579), LO-PRO™-1 iodide (567/580), NeuroTrace™ 435/455, NeuroTrace™ 500/525, NeuroTrace™ 515/535, NeuroTrace™ 530/615, NeuroTrace™ 640/660, OliGreen, PicoGreen® ssDNA, PicoGreen® dsDNA, POPO™-4 iodide (434/456), POPO™-3 iodide (534/570), PO-PRO™-1 iodide (435/455), POPRO™-3 iodide (539/567), propidium iodide, RiboGreen®, SlowFade®, SlowFade® Light, SYBR® Green I, SYBR® Green II, SYBR® Gold, SYBR® 101, SYBR® 102, SYBR® 103, SYBR® DX, TO-PRO®-1, TO-PRO®-3, TO-PRO®-5, TOTO®-1, TOTO®-3, YO-PRO®-1 (oxazole yellow), YO-PRO®-3, YOYO™-1, YOYO™-3, TO, SYTOX® Blue, SYTOX® Green, SYTOX® Orange, SYTO® 9, SYTO® BC, SYTO® 40, SYTO® 41, SYTO® 42, SYTO® 43, SYTO® 44, SYTO® 45, SYTO® Blue, SYTO® 11, SYTO® 12, SYTO® 13, SYTO® 14, SYTO® 15, SYTO® 16, SYTO® 20, SYTO® 21, SYTO® 22, SYTO® 23, SYTO® 24, SYTO® 25, SYTO® Green, SYTO® 80, SYTO® 81, SYTO® 82, SYTO® 83, SYTO® 84, SYTO® 85, SYTO® Orange, SYTO® 17, SYTO® 59, SYTO® 60, SYTO® 61, SYTO® 62, SYTO® 63, SYTO® 64, SYTO® Red, netropsin, distamycin, acridine orange, 3,4-benzopyrene, thiazole orange, TOMEHE, daunomycin, acridine, pentyl-TOTAB, and butyl-TOTIN. Asymmetric cyanine dyes may be used as the polynucleotide-specific dye. Other dyes of interest include those described by Geierstanger, B. H. and Wemmer, D. E., Annu. Rev. Vioshys. Biomol. Struct. 1995, 24, 463-493, by Larson, C. J. and Verdine, G. L., Bioorganic Chemistry Nucleic Acids, Hecht, S. M., Ed., Oxford University Press: New York, 1996; pp 324-346, and by Glumoff, T. and Goldman, A. Nucleic Acids in Chemistry and Biology, 2.sup.nd ed., Blackburn, G. M. and Gait, M. J., Eds., Oxford University Press: Oxford, 1996, pp 375-441. The polynucleotide-specific dye may be an intercalating dye, and may be specific for double-stranded polynucleotides. Other dyes and fluorophores are described at www.probes.com (Molecular Probes, Inc.).

The term “green fluorescent protein” refers to both native Aequorea green fluorescent protein and mutated versions that have been identified as exhibiting altered fluorescence characteristics, including altered excitation and emission maxima, as well as excitation and emission spectra of different shapes (Delagrave, S. et al. (1995) Bio/Technology 13:151-154; Heim, R. et al. (1994) Proc. Natl. Acad. Sci. USA 91:12501-12504; Heim, R. et al. (1995) Nature 373: 663-664). Delgrave et al. isolated mutants of cloned Aequorea victoria GFP that had red-shifted excitation spectra. Bio/Technology 13:151-154 (1995). Heim, R. et al. reported a mutant (Tyr66 to His) having a blue fluorescence (Proc. Natl. Acad. Sci. (1994) USA 91:12501-12504).

In some embodiments, the water-soluble fluorescent polymer or water-soluble fluorescent polymer complex has one or more, two or more, three or more, four or more, 1-25, 2-20, 3-15, or 4-12 chromophores, such as chromophores, fluorophores, lumophores, acceptor dyes, and the like.

The term “linker,” “linker moiety,” or “linkage” refers to a “linking moiety” that connects two groups and has a backbone of 100 atoms or less in length. A linker moiety or linkage (e.g., L, L¹, L², L³, and the like) may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some embodiments, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some embodiments, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

A linker moiety can be attached to “L” or to “A”, as taught in US Published Application No. 2020/0190253A1, which is incorporated herein by reference in its entirety. A linker moiety can comprise a sulfonamide, disulfonamide, a selenomide, a sulfinamide, a sultam, a disulfinamide, an amide, a seleninamide, a phosphonamide, a phosphinamide, a phosphonamidate, or a secondary amine.

As described therein, and as each pertains to a linker moiety, the term “sulfonamide,” refers to a moiety —S(O)₂NR—; the term “disulfonamide,” refers to a moiety —S(O)₂NRS(O)₂—; the term “selenonamide,” refers to a moiety —Se(O)₂NR—; the term “sulfinamide,” refers to a moiety —S(O)NR—; the term “disulfinamide,” refers to a moiety —S(O)NRS(O)—; the term “seleninamide,” refers to a moiety —Se(O)NR—; the term “phosphonamide,” refers to a moiety —NR—PR(O)NR—; the term “phosphinamide,” refers to a moiety —PR(O)NR—; and the term “phosphonamidate,” refers to a moiety —O—PR(O)NR—; and the term “sultam” refers to a cyclic sulfonamide (e.g., wherein the R group is bonded to the sulfur atom via an alkylene moiety); wherein for each term the R group is independently H, alkyl, haloalkyl, or aryl.

The subject water-soluble fluorescent polymers feature termini on the conjugated polymer chains that can include a functional group that provides for bioconjugation. In some cases, such functionality is referred to as an end linker. With these end linkers, a covalent bond can be formed to attach a biomolecule such as a protein, peptide, affinity ligand, antibody, antibody fragment, polynucleotide, or aptamer. For example, polymeric dye-labeled antibodies find use in flow cytometry as reagents exhibiting high brightness. Additionally, orthogonal functional groups can be installed along the conjugated polymer chain that can be used for either bioconjugation or the attachment of acceptor signaling chromophores in donor acceptor polymeric tandem dyes.

The phrase “conjugated water-soluble fluorescent polymer” refers to a water-soluble fluorescent polymer having a binding agent conjugated thereto.

“ [symbol] ” represents either a single or double bond.

The phrase “binding agent” refers to any molecule or complex of molecules capable of specifically binding to a target analyte.

A binding agent of the invention includes for example, a protein (e.g., an antibody or an antibody fragment), a small organic molecule, a carbohydrate (e.g., a polysaccharide), an oligonucleotide, a polynucleotide, a lipid, an affinity ligand, an aptamer, or the like. In some embodiments, the binding agent is an antibody, antibody fragment, protein, peptide, affinity ligand, antibody, antibody fragment, carbohydrate, lipid, nucleic acid, or an aptamer. In some embodiments, the binding agent is an antibody or fragment thereof. Specific binding in the context of the present invention refers to a binding reaction which is determinative of the presence of a target analyte in the presence of a heterogeneous population. Thus, under certain assay conditions, the specified binding agents bind preferentially to a particular protein or isoform of the particular protein and do not bind in a significant amount to other proteins or other isoforms present in the sample.

In some cases, the antibody includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. Aspects of IVIG are described, for example, in US. Pat. Appl. Pub. Nos. 2010/0150942; 2004/0101909; 2013/0177574; 2013/0108619; and 2013/0011388.

In some cases, the antibody is a monoclonal antibody of a defined sub-class (e.g., IgG1, IgG2, IgG3, or IgG4). If combinations of antibodies are used, the antibodies can be from the same subclass or from different subclasses. For example, the antibodies can be IgG1 antibodies. In some embodiments, the monoclonal antibody is humanized.

As used herein, the term “specific binding” or “specifically binds” refers to the ability of a binding agent to preferentially bind to a particular target analyte that is present, e.g., in a homogeneous mixture of different analytes. In some instances, a specific binding interaction will discriminate between desirable and undesirable analytes in a sample with a specificity of 10-fold or more for a desirable analyte (i.e., target analyte) over an undesirable analyte, such as 100-fold or more, or 1000-fold or more. In some cases, the affinity between a capture agent and analyte when they are specifically bound in a capture agent/analyte complex is at least 10⁻⁸ M, at least 10⁻⁹ M, such as up to 10⁻¹⁰ M.

The phrase “water-soluble fluorescent polymer complex” refers to a water-soluble fluorescent polymer of the invention conjugated with a binding agent.

The phrase “protected group” (also referred to as “protecting group”) refers to a chemical moiety that renders an functional group unreactive, but is also removable so as to restore the functional group. Examples of amine protecting groups include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. These and other protecting groups for amines, carboxylic acids, alcohols, and further functional groups can be added to and removed from polymers of the present disclosure using known techniques as described, for example, by Green and Wuts (Protective Groups in Organic Synthesis, 4^{th}Ed. 2007, Wiley-Interscience, New York).

The term “substrate” refers to a solid material having a variety of configurations. The substrate can be, for example, a sheet, bead, or other structure, such as a plate with wells, a polymer, particle, a semiconductor surface, nanotubes, a fibrous mesh, hydrogels, porous matrix, a pin, a microarray surface, a chromatography support, and the like. In some instances, the substrate is selected from the group consisting of a particle, a planar solid substrate, a fibrous mesh, a hydrogel, a porous matrix, a pin, a microarray surface and a chromatography support.

The term “water” as used herein refers to any aqueous solution that is primarily water and is compatible with physiological conditions. In some instances, the aqueous solution contains more than 50% water, such as more than 60% water, more than 70% water, more than 80% water, more than 90% water, or more than 95% water. The term “water” includes, for example, biological buffers and other aqueous solutions that may contain additives such as salts, detergents, stabilizers, and other water-soluble components, for example, sugars, proteins, amino acids, and nucleotides. In some instances, “water” may be an aqueous solution containing up to 10% miscible organic solvent (e.g., up to 10% DMSO in water). The term “water” does not include pure solvents or solvent combinations different from water, such as pure alcohols, for example pure methanol or ethanol, pure ethers, for example pure diethyl ether or tetrahydrofuran, or any other pure solvent either miscible or not miscible with water.

The term “water-solubilizing moiety” as used herein by itself or part of another group refers to any hydrophilic group that is well solvated in aqueous environments, for example such as under physiological conditions, and is capable of increasing the water solubility of the molecule to which it is attached. The increase in water solubility of the molecule can vary depending upon the moiety attached. In some instances, the increase in water solubility (as compared to the solubility of the molecule without the moiety attached) is 2 fold or more, 5 fold or more, 10 fold or more, 25 fold or more, 50 fold or more, or 100 fold or more. “Water-solubilizing moiety” includes moieties, such as, but not limited to, PEG groups, carboxy groups including but not limited to carboxylic acids, carboxylates, polyvinyl alcohol, glycols, peptides, polyphosphates, polyalcohols, sulfonates, phosphonates, boronates, amines, ammoniums, sulfoniums, phosphonium, alcohols, zwitterionic derivatives, carbohydrates, nucleotides, polynucleotides, polyethylene glycols (PEGs), substituted PEG groups, substituted carboxy groups including but not limited to substituted carboxylic acids and substituted carboxylates, substituted glycols, substituted peptides, substituted polyphosphates, substituted polyalcohols, substituted sulfonates, substituted phosphonates, substituted boronates, substituted amines, substituted ammoniums, substituted sulfoniums, substituted phosphonium, alcohols, substituted zwitterionic derivatives, substituted carbohydrates, substituted nucleotides, substituted polynucleotides, and combinations thereof.

The term “water-soluble polymer” (WSP) as used herein refers to a polymer having solubility in “water” as used herein of 1 mg/mL or more, such as 3 mg/mL or more, 10 mg/mL or more, 20 mg/mL or more, 30 mg/mL or more, 40 mg/mL or more, 50 mg/mL or more, 60 mg/mL or more, 70 mg/mL or more, 80 mg/mL or more, 90 mg/mL or more, 100 mg/mL or more, or even more. It is understood that water soluble polymers may, under certain conditions, form discrete water-solvated nanoparticles in aqueous systems and can be resistant to aggregation.

### III. Compositions

### Polymers

The water-soluble fluorescent polymers of the invention have the structure as shown in Formula I:

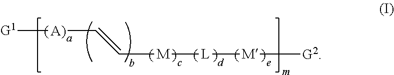

A can be

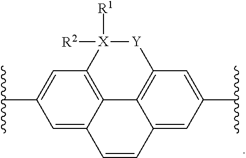

### A can be

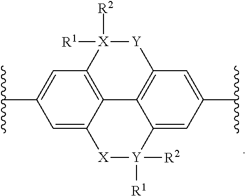

### A can be

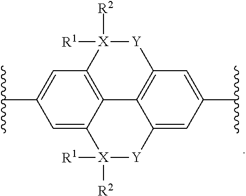

### A can be

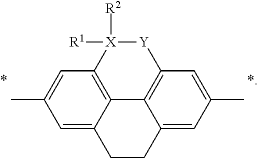

### A can be

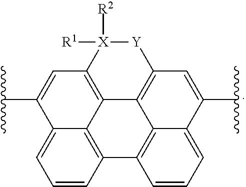

### A can be

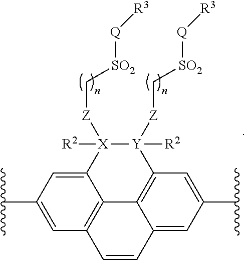

### A can be

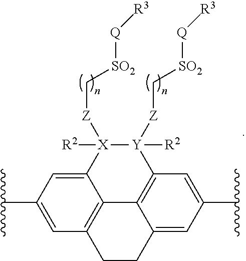

### A can be

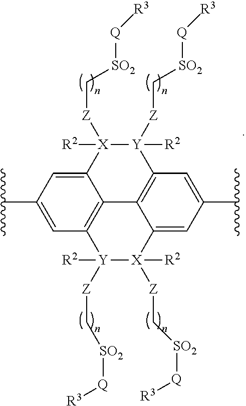

### A can be

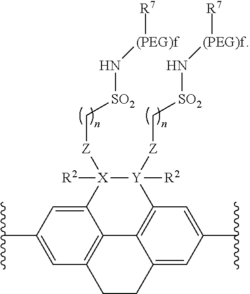

### A can be

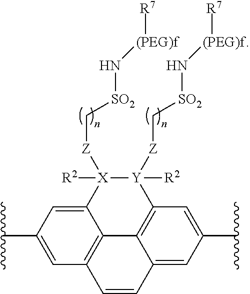

### A can be

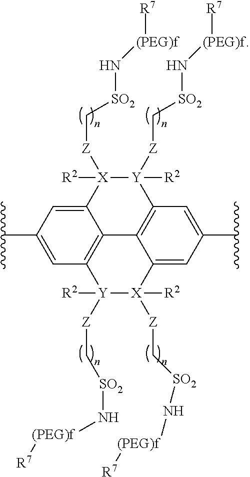

### A can be

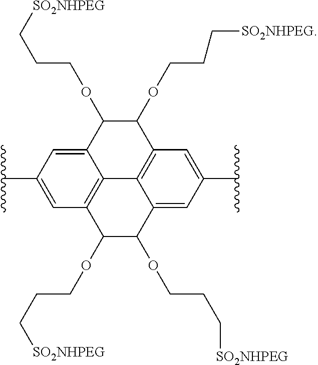

### A can be

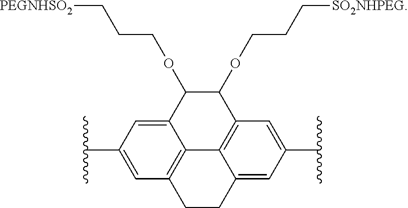

### A can be

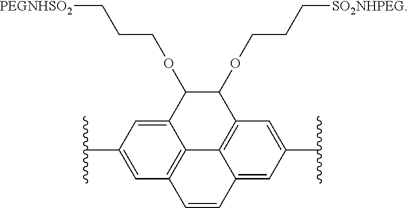

X can be C. X can be Si. Each X can be different. Both X can be the same.

Y can be CH₂. Y can be CR¹R². Y can be SiR¹R². Each Y can be different. Each Y can be the same.

R¹ can be a water-solubilizing moiety. R¹ can be alkene. R¹ can be C₁₋₁₀-alkene. R¹ can be methene, ethene, n-propene, i-propene, n-butene, i-butene, or t-butene. R¹ can be alkyne. R¹ can be C₁₋₁₀-alkyne. R¹ can be methyne, ethyne, n-propyne, i-propyne, n-butyne, i-butyne, or t-butyne. R¹ can be cycloalkyl. R¹ can be haloalkyl. R¹ can be (hetero) aryloxy. R¹ can be (hetero) arylamino. R¹ can be PEG. R¹ can be carboxylic acid. R¹ can be ammonium alkyl salt. R¹ can be ammonium alkyloxy salt. R¹ can be ammonium oligoether salt. R¹ can be sulfonate alkyl salt. R¹ can be sulfonate alkoxy salt. R¹ can be sulfonate oligoether salt. R¹ can be sulfonamido oligoether. R¹ can be sulfonamide. R¹ can be sulfinamide. R¹ can be phosphonamidite. R¹ can be phosphinamide. R¹ can be

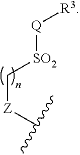

### R¹ can be

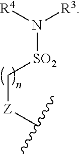

### R¹ can be

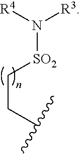

### R¹ can be

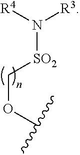

### R¹ can be

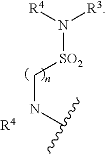

### R¹ can be

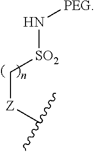

### R¹ can be

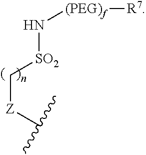

### R¹ can be

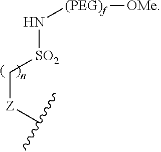

### R¹ can be

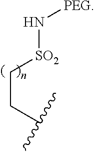

### R¹ can be

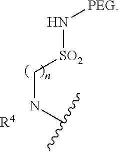

### R¹ can be

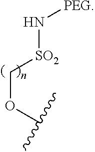

### R¹ can be

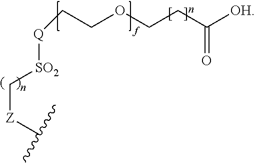

### R¹ can be

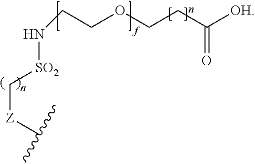

### R¹ can be

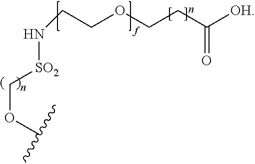

### R¹ can be

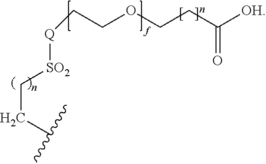

### R¹ can be

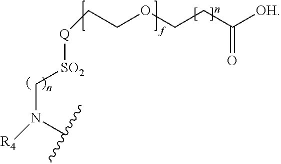

### R¹ can be

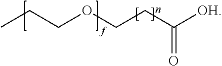

### R¹ can be

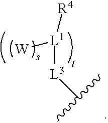

### R¹ can be

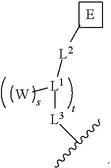

Each instance of R¹ can be different. All instances of R¹ can be the same.

R² can be a water-solubilizing moiety. R² can be a linker moiety. R² can be C₁₋₁₀-alkyl. R² can be methyl, ethyl, n-propyl, i-propyl, n-butyl, i-butyl, or t-butyl. R² can be alkene. R² can be C₁₋₁₀-alkene. R² can be methene, ethene, n-propene, i-propene, n-butene, i-butene, or t-butene. R² can be alkyne. R² can be C₁₋₁₀-alkyne. R² can be methyne, ethyne, n-propyne, i-propyne, n-butyne, i-butyne, or t-butyne. R² can be cycloalkyl. R² can be haloalkyl. R² can be alkoxy. R² can be (hetero)aryloxy. R² can be aryl. R² can be (hetero)arylamino. R² can be PEG. R² can be sulfonamide-PEG. R² can be phosphoramide-PEG. R² can be ammonium alkyl salt. R² can be ammonium alkyloxy salt. R² can be ammonium oligoether salt. R² can be sulfonate alkyl salt. R² can be sulfonate alkoxy salt. R² can be sulfonate oligoether salt. R² can be sulfonamido oligoether. R² can be sulfonamide. R² can be sulfinamide. R² can be phosphoamidite. R² can be phosphinamide. R² can be

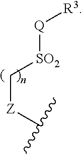

### R² can be

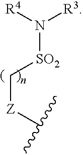

### R² can be

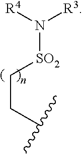

### R² can be

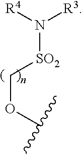

### R² can be

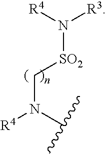

### R² can be

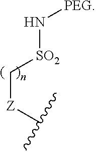

### R² can be

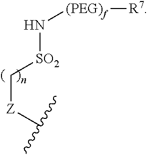

### R² can be

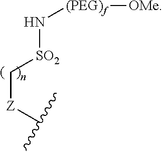

### R² can be

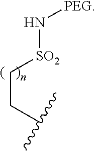

### R² can be

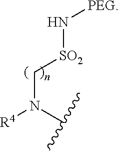

### R² can be

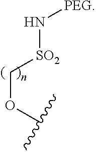

### R² can be

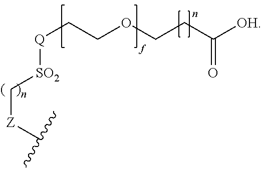

### R² can be

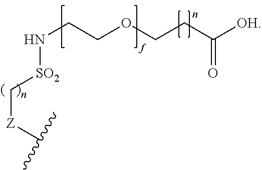

### R² can be

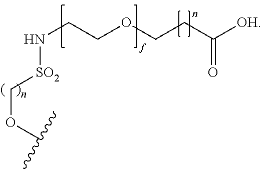

### R² can be

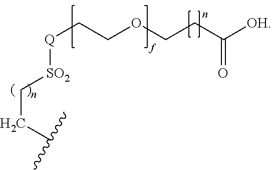

### R² can be

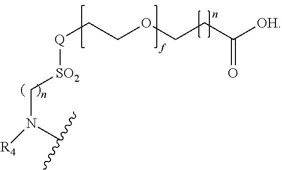

### R² can be

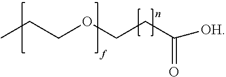

### R² can be

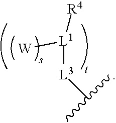

### R² can be

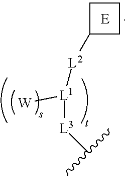

Each instance of R² can be different. All instances of R² can be the same.

R³ can be a WSP. R³ can be PEG or modified PEG polymer. The modified PEG polymer can be of 6-30 monomeric units, such as 6-24 or 10-30, 10-24 or 10-20, 12-24, 12-20, 12-16 or 16-20 monomeric unit. The modified PEG polymer can be terminated with a carboxlic acid or carboxylate ester. R³ can be

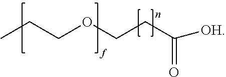

R³ can be alkyl. R³ can be a polymer comprising 6-24 monomeric units.

R⁴ can be H. R⁴ can be a PEG group. R⁴ can be a water-solubilizing moiety. R⁴ can be a linker moiety. R⁴ can be a chromophore or a linked chromophore. R⁴ can be a functional group or a linked functional group. R⁴ can be a functional tag or a linked functional tag. R⁴ can be a substrate or a linked substrate. R⁴ can be a binding agent or a linked binding agent. R⁴ can be L²-E. R⁴ can be halogen. R⁴ can be hydroxyl. R⁴ can be alkylamino. R⁴ can be substituted or unsubstituted C₁-C₁₂ alkyl. R⁴ can be C₂-C₁₂ alkene. R⁴ can be C₂-C₁₂ alkyne. R⁴ can be C₃-C₁₂ cycloalkyl. R⁴ can be C₁-C₁₂ haloalkyl. R⁴ can be C₁-C₁₂ alkoxy. R⁴ can be C₂-C₁₈ (hetero)aryloxy. R⁴ can be C₂-C₁₈ (hetero)arylamino. R⁴ can be (CH₂)_{x′}(OCH₂—CH₂)_{y′}OR⁹, wherein each R⁹ is C₁-C₈ alkyl, x′ is independently an integer from 0-20 and each y′ is independently an integer from 0-50. R⁴ can be Z—(CH₂)ₙ—SO₂—Q—R³. R⁴ can be a C₂-C₁₈ (hetero)aryl group. R⁴ can be an amide. R⁴ can be an amine. R⁴ can be a carbamate. R⁴ can be carboxylic acid. R⁴ can be carboxylate ester. R⁴ can be a maleimide. R⁴ can be an activated ester. R⁴ can be N-hydroxysuccinimidyl. R⁴ can be hydrazine. R⁴ can be hydrazide. R⁴ can be hydrazone. R⁴ can be an azide. R⁴ can be an alkyne. R⁴ can be an aldehyde. R⁴ can be a thiol. Independently, each R⁴ can comprise a protected group. Independently, each R⁴ can optionally be conjugated to a substrate, chromophore, functional tag, or binding agent.

Q can be a bond. Q can be NR⁴. Q can be —CH₂.

Z can be CH₂. Z can be O. Z can be NR⁴.

W can be a water-solubilizing moiety. W can be a water-solubilizing moiety selected from the group consisting of an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, an oligo(ethylene glycol), and a poly(ethylene glycol).

E can be a chromophore. E can be an acceptor dye. E can be a substrate. E can be a functional tag. E can be a binding agent.

L¹ can be a linker moiety. L¹ can be a linker moiety independently selected from the group consisting of a covalent bond, substituted or unsubstituted C₁₋₈ alkylene, substituted or unsubstituted 2- to 8-membered heteroalkylene, oligo(ethylene glycol), ether, thioether, tertiary amine, a straight or branched alkylene group, and cyclic group. L¹ can be a sulfonamide. L¹ can be a sulfinamide. L¹ can be a disulfonamide. L¹ can be a disulfinamide. L¹ can be a sultam. L¹ can be an amide. L¹ can be a secondary amine. L¹ can be a phosphonamide. L¹ can be a phosphinamide. L¹ can be a phosphonamidite. L¹ can be a selenonamide. L¹ can be a seleninamide. L¹ can independently be a sulfonamide, an amide, a secondary amine, or a phosphonamide

L² can be a linker moiety. L² can be linker moiety independently selected from the group consisting of a covalent bond, hydrogen, a protecting group, substituted or unsubstituted C₁₋₈ alkylene, substituted or unsubstituted 2- to 8-membered heteroalkylene, oligo(ethylene glycol), ether, thioether, tertiary amine, a straight or branched alkylene group, and cyclic group. L² can be a linear or branched, saturated or unsaturated C₁₋₃₀ alkylene group, wherein one or more carbon atoms in the C₁₋₃₀ alkylene group is optionally and independently replaced by O, S, NR^{a}; or two or more groupings of adjacent carbon atoms in the C₁₋₃₀ alkylene are optionally and independently replaced by —NR^{a}(CO)— or —(CO)NR^{a}—, wherein each R^{a} is independently selected from the group consisting of H and substituted or unsubstituted C₁₋₆ alkyl.

L³ can be a linker moiety. L³ can be a covalent bond. L³. L³ can be a linker moiety independently selected from the group consisting of a covalent bond, substituted or unsubstituted C₁₋₈ alkylene, substituted or unsubstituted 2- to 8-membered heteroalkylene, oligo(ethylene glycol), ether, thioether, tertiary amine, a straight or branched alkylene group, and cyclic group. L³ can be a trivalent arylalkyl moiety having a first point of attachment to a first L¹ moiety; a second point of attachment to a second L¹ moiety; and a third point of attachment to an A monomer.

R⁷ can be H. R⁷ can be C₁-C₁₂ alkyl. R⁷ can be C₂-C₁₂ alkene. R⁷ can be C₂-C₁₂ alkyne. R⁷ can be C₃-C₁₂ cycloalkyl. R⁷ can be C₁-C₁₂haloalkyl. R⁷ can be C₁-C₁₂ alkoxy. R⁷ can be C₂-C₁₈ (hetero)aryloxy. R⁷ can be C₂-C₁₈ (hetero)arylamino. R⁷ can be C₂-C₁₂ carboxylic acid. R⁷ can be C₂-C₁₂ carboxylate ester. R⁷ can be C₁-C₁₂ alkoxy.

f can independently be an integer from 0 to 50.

n can independently be an integer from 1 to 20.

s can be 1 or 2.

t can be 1, 2, 3 or 4.

The water-soluble fluorescent polymers of the present disclosure can contain polymer modifying units, represented in Formula I as M and M′, that are capable of altering the polymer band gap. The polymer modifying units M and M′ can each independently be an absorbance modifying co-monomer. M and/or M′ can each independently be selected from the group consisting of a substituted or unsubstituted 1,4-phenyl, a substituted or unsubstituted 1,3-phenyl, a substituted or unsubstituted 4,4′-biphenyl, a substituted or unsubstituted 2,5-pyridyl, a substituted or unsubstituted 2,6-pyridyl, substituted or unsubstituted 9,10-dihydrophenanthrene, substituted or unsubstituted fluorene, substituted or unsubstituted binaphthyl. M and/or M′ can be evenly or randomly distributed along the polymer main chain. Each M and/or M′ is optional. M and/or M′ can be optionally substituted with one or more optionally substituted R¹, R², R³, or R⁴ groups.

M and/or M′ can be

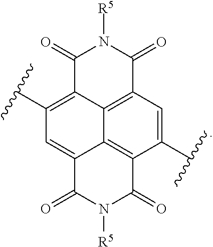

M and/or M′ can be

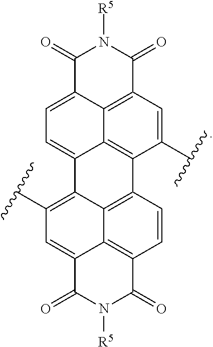

M and/or M′ can be

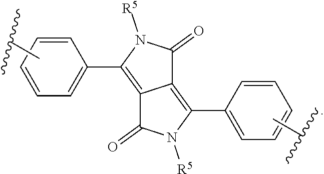

M and/or M′ can be

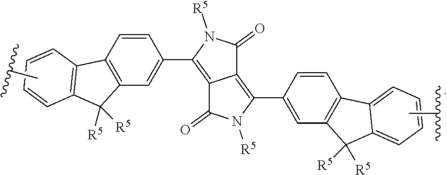

M and/or M′ can be

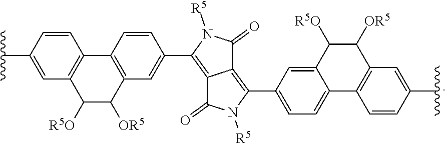

M and/or M′ can be

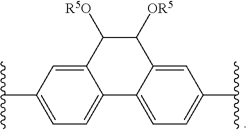

M and/or M′ can be

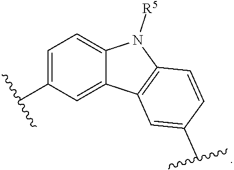

M and/or M′ can be

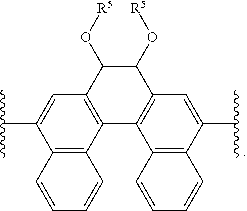

M and/or M′ can be

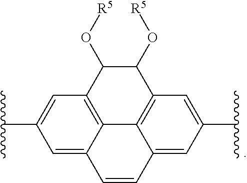

M and/or M′ can be

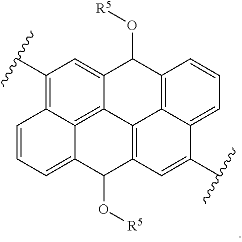

M and/or M′ can be

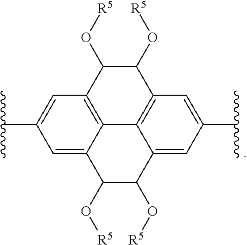

M and/or M′ can be

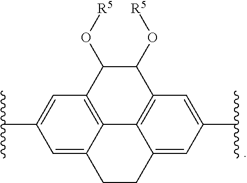

M and/or M′ can be

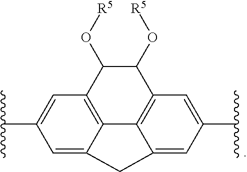

M and/or M′ can be

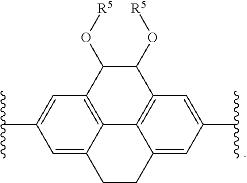

M and/or M′ can be

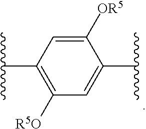

M and/or M′ can be

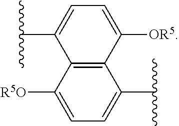

M and/or M′ can be

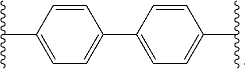 [SMILES: *c1ccc(-c2ccc(*)cc2)cc1]

M and/or M′ can be

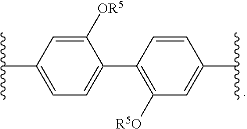

M and/or M′ can be

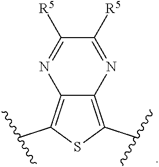

M and/or M′ can be

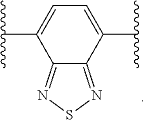 [SMILES: *c1ccc(*)c2nsnc12]

M and/or M′ can be

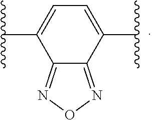 [SMILES: *c1ccc(*)c2nonc12]

M and/or M′ can be

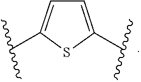 [SMILES: *c1ccc(*)s1]

M and/or M′ can be

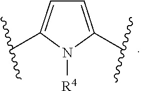

### M can be

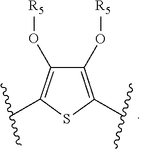

M and/or M′ can

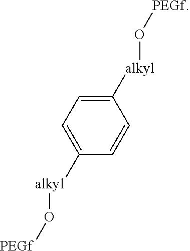

### M can be

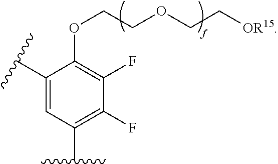

M and/or M′ can be

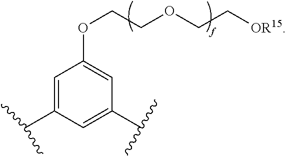

M and/or M′ can be

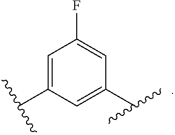 [SMILES: *c1cc(*)cc(F)c1]

### M can be

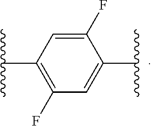 [SMILES: *c1cc(F)c(*)cc1F]

M and/or M′ can be

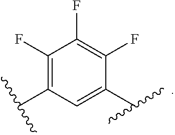 [SMILES: *c1cc(*)c(F)c(F)c1F]

M and/or M′ can be

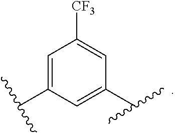 [SMILES: *c1cc(*)cc(C(F)(F)F)c1]

M and/or M′ can be

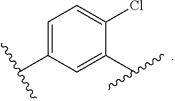 [SMILES: *c1ccc(Cl)c(*)c1]

M and/or M′ can be

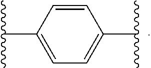 [SMILES: *c1ccc(*)cc1]

### M can be

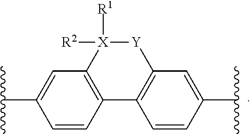

M and/or M′ can be

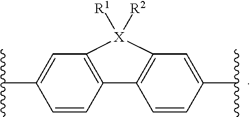

M and/or M′ can be

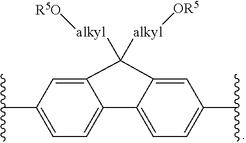

M and/or M′ can be

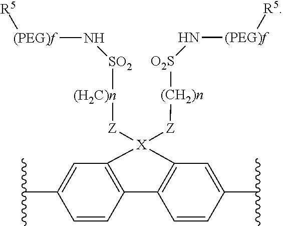

M and/or M′ can be

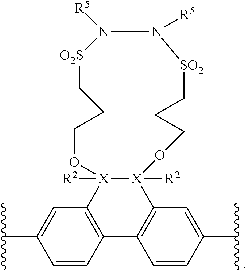

M and/or M′ can be

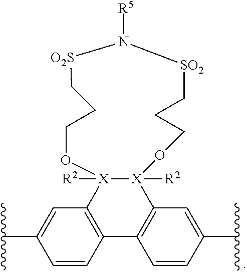

M and/or M′ can be

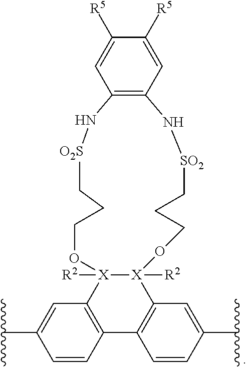

M and/or M′ can be

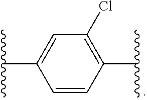 [SMILES: *c1ccc(*)c(Cl)c1]

M and/or M′ can be

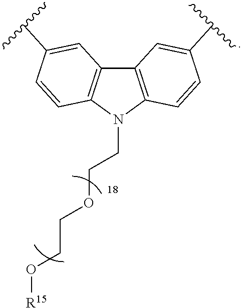

M and/or M′ can be

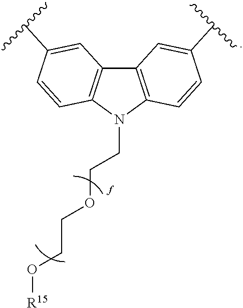

M and/or M′ can be

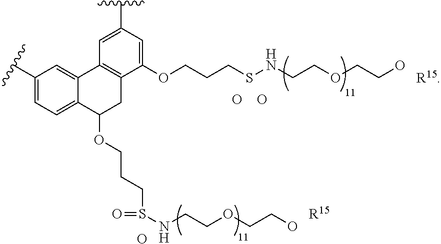

M and/or M′ can be

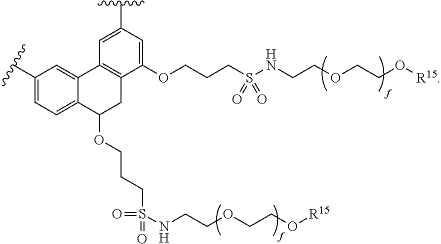

M and/or M′ can be

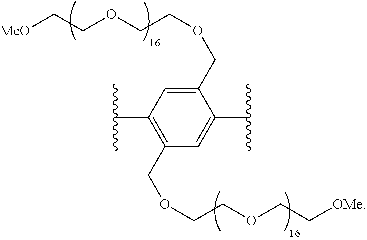

M and/or M′ can be

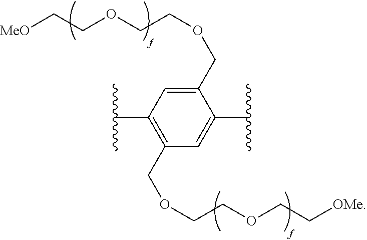

M and/or M′ can be

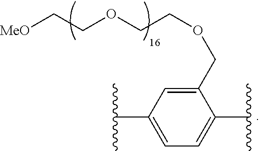

M and/or M′ can be

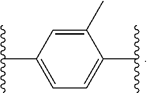 [SMILES: *c1ccc(*)c(C)c1]

M and/or M′ can be

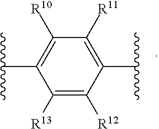

M and/or M′ can be

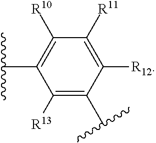

M and/or M′ can be

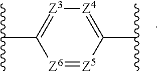

M and/or M′ can be

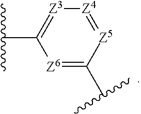

M and/or M′ can be

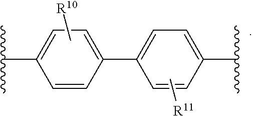

M and/or M′ can be

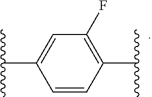 [SMILES: *c1ccc(*)c(F)c1]

M and/or M′ can be

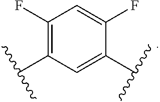 [SMILES: *c1cc(*)c(F)cc1F]

M and/or M′ can be

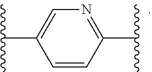 [SMILES: *c1ccc(*)nc1]

M and/or M′ can be

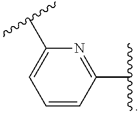 [SMILES: *c1cccc(*)n1]

M and/or M′ can be

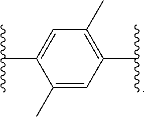 [SMILES: *c1cc(C)c(*)cc1C]

M and/or M′ can be

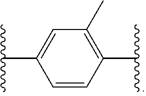 [SMILES: *c1ccc(*)c(C)c1]

M and/or M′ can be

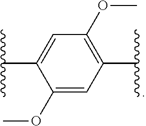 [SMILES: *c1cc(OC)c(*)cc1OC]

M and/or M′ can be

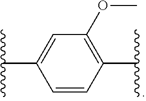 [SMILES: *c1ccc(*)c(OC)c1]

M and/or M′ can be

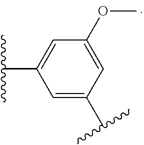 [SMILES: *c1cc(*)cc(OC)c1]

M and/or M′ can be

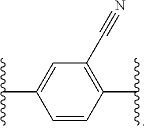 [SMILES: *c1ccc(*)c(C#N)c1]

M and/or M′ can be

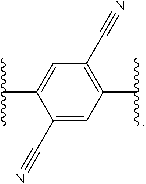 [SMILES: *c1cc(C#N)c(*)cc1C#N]

M and/or M′ can be

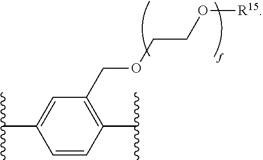

M and/or M′

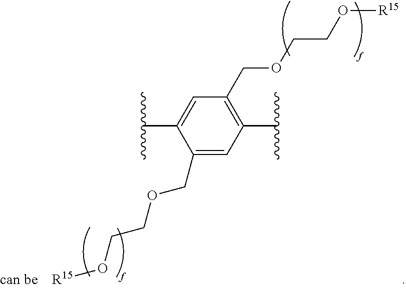

M and/or M′ can be

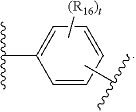

M and/or M′ can be

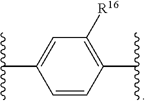

M and/or M′ can be

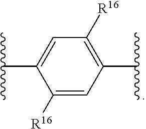

M and/or M′ can be

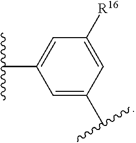

M and/or M′ can be

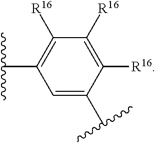

M and/or M′ can be

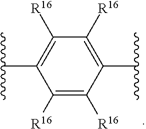

M and/or M′ can be

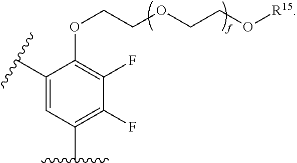

M and/or M′ can be

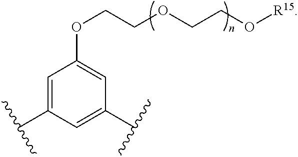

M and/or M′ can be substituted. M and/or M′ can be terminated with a functional group selected from amine, carbamate, carboxylic acid, carboxylate ester, maleimide, activated ester, N-hydroxysuccinimidyl, hydrazine, hydrazide, hydrazone, azide, alkyne, aldehyde, thiol, amide, sulfonamide, ether, thioether, thiocarbamate, hydroxyl, iodoacetyl, hydrazido, hydrazino, ketone, phosphine, epoxide, urea, thiourea, thioester, imine, disulfides, and protected groups thereof, optionally conjugated to a substrate, chromophore, acceptor dye, functional tag, or binding agent. Each of R¹, R², R³ f, and n can be as described above. Each Z³-Z⁶ can independently be CR¹⁴ or N, wherein at least one Z³-Z⁶ is N. Each R¹⁰-R¹⁴ can independently be selected from the group consisting of hydrogen, halogen, cyano, alkoxy, substituted alkoxy, alkyl, substituted alkyl, alkene, substituted alkene, alkyne, azide, PEG moiety, substituted PEG moiety, and water solubilizing moiety. Each R¹⁵ can independently be H or lower alkyl. Each R¹⁶ can independently be selected from the group consisting of H, F, Cl, —CF₃, and —(CH₂CH₂)OR¹⁵.

R⁵ can be halogen. R⁵ can be hydroxyl. R⁵ can be C₁-C₁₂ alkyl. R⁵ can be C₂-C₁₂ alkene. R⁵ can be C₂-C₁₂ alkyne. R⁵ can be C₃-C₁₂ cycloalkyl. R⁵ can be C₁-C₁₂ haloalkyl. R⁵ can be C₁-C₁₂ alkoxy. R⁵ can be C₂-C₁₈ (hetero)aryloxy. R⁵ can be C₂-C₁₈ (hetero)arylamino. R⁵ can be carboxylic acid. R⁵ can be carboxylate ester. R⁵ can be (CH₂)_{x′}(OCH₂—CH₂)_{y′}OCH₃. R⁵ can be C₂₋₁₈ (hetero)aryl group.

x′ can be an integer from 0-20. x′ can be an integer from 0-10. x′ can be an integer from 1-4.

y′ can be an integer from 0-50. y′ can be an integer from 0-40. y′ can be an integer from 0-30. y′ can be an integer from 0-20. y′ can be an integer from 0-10. y′ can be an integer from 1-4.

The water-soluble fluorescent polymers of the present disclosure can also contain linkers represented in Formula I as L. Each optional L can be evenly or randomly distributed along the polymer main chain. L can be —(CH₂)ₚ—O— wherein p is from 1 to 12, e.g., 1 to 6. L can be —O—(CH₂)ₚ— wherein p is from 1 to 12, e.g., 1 to 6. L can be —(CH₂)ₚ— wherein p is from 1 to 12, e.g., 1 to 6. L can be —O—. L can be C₁-C₁₂-alkyl linker, e.g., a C₁-C₆-alkyl linker, wherein one or more backbone atoms are optionally substituted with a heteroatom. L can be an aryl group. L can be a heteroaryl group. When L is an aryl or heteroaryl group, it can be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof. Each L can optionally be conjugated to a substrate, chromophore, acceptor dye, functional tag, or binding agent.

### L can be

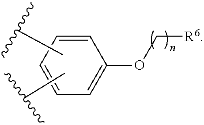

### L be

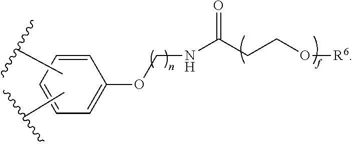

### L can be

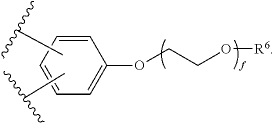

### L can be

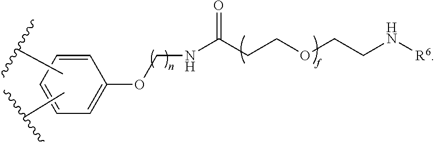

### L can be

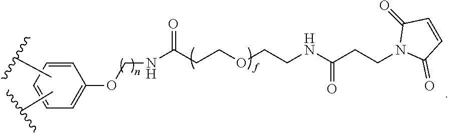

### L can be

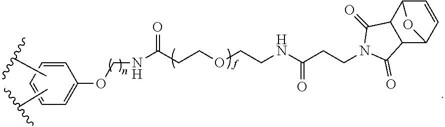

### L can be

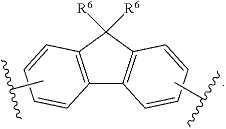

### L can be

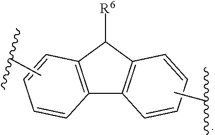

### L can be

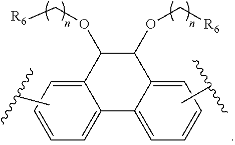

### L can be

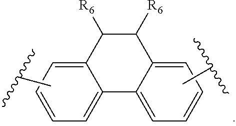

### L can be

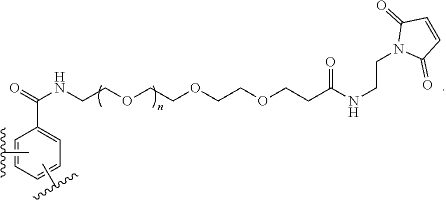

### L can be

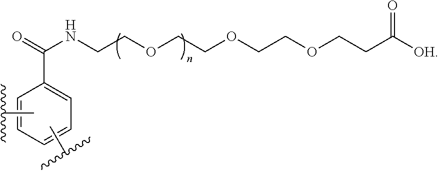

### L can be

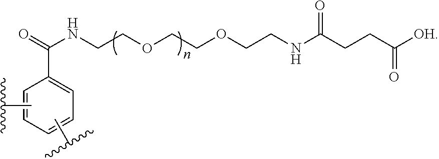

### L can be

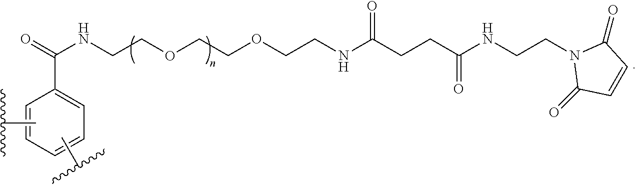

### L can be

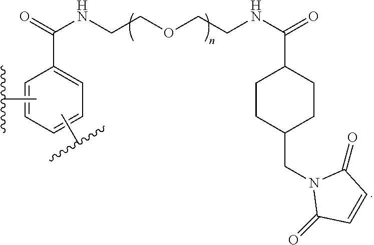

### L can be

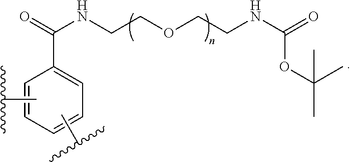

### L can be

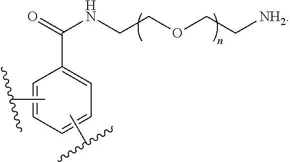

Each R⁶ is independently selected. R⁶ can be H. R⁶ can be OH. R⁶ can be SH. R⁶ can be NHCOO-t-butyl. R⁶ can be (CH₂)ₙCOOH. R⁶ can be (CH₂)ₙCOOR¹⁵. R⁶ can be (CH₂)ₙ(CH₂CH₂O)_{f}COOH. R⁶ can be —(CH₂)ₙCOOCH₃. R⁶ can be —(CH₂)ₙNH₂. R⁶ can be —(CH2)nNH(CH2)nCH3. R6 can be —(CH2)nNHCOOH. R6 can be —(CH₂)ₙNHCO(CH₂)ₙCO(CH₂)ₙCH₃. R⁶ can be —(CH₂)ₙNHCOO(CH₂)ₙCH₃. R⁶ can be —(CH₂)ₙNHCOOC(CH₃)₃. R⁶ can be —(CH₂)ₙNHCO(C₃-C₁₂)cycloalkyl. R⁶ can be (CH₂)ₙNHCO(CH₂CH₂O)_{f}OR¹⁵. R⁶ can be —(CH₂)ₙNHCO(CH₂CH₂O)_{f}. R⁶ can be —(CH₂)ₙNHCO(CH₂)ₙCOOH. R⁶ can be —(CH₂)ₙNHCO(CH₂)ₙCOO(CH₂)ₙCH₃. R⁶ can be (CH₂)ₙ(OCH₂CH₂)_{f}OR¹⁵. R⁶ can be —(CH₂)ₙ(OCH₂CH₂)_{f}OCH₃. R⁶ can be N-maleimide. R⁶ can be halogen, C₂-C₁₂ alkene. R⁶ can be C₂-C₁₂ alkyne. R⁶ can be C₃-C₁₂ cycloalkyl. R⁶ can be C₁-C₁₂ halo alkyl. R⁶ can be C₁-C₁₂ (hetero)aryl. R⁶ can be C₁-C₁₂ (hetero)arylamino. R⁶ can be benzyl optionally substituted with one or more halogen, hydroxyl, C₁-C₁₂ alkoxy, or (OCH₂CH₂)_{f}OCH₃. R⁶ can be carboxylic acid. R⁶ can be carboxylate ester. R⁶ can be

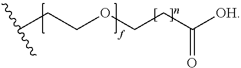

### R⁶ can be

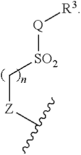

### R⁶ can be

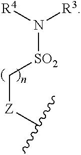

### R⁶ can be

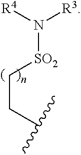

### R⁶ can be

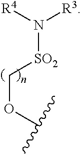

### R⁶ can be

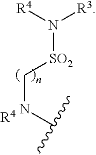

### R⁶ can be

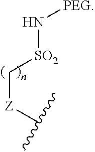

### R⁶ can be

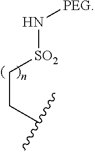

### R⁶ can be

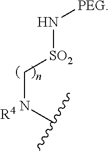

### R⁶ can be

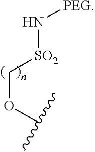

### R⁶ can be

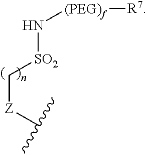

### R⁶ can be

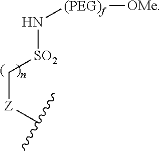

### R⁶ can be

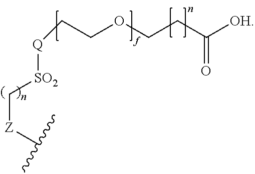

### R⁶ can be

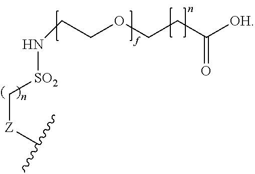

### R⁶ can be

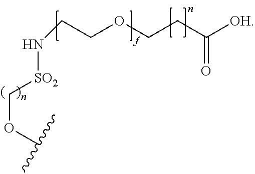

### R⁶ can be

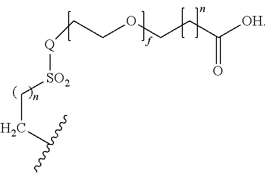

### R⁶ can be

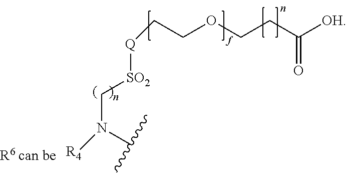

### R⁶ can be

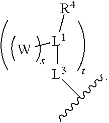

### R⁶ can be

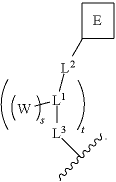

R³, R⁴, f and n can be as described above.

The water-soluble fluorescent polymers of the present disclosure also contain capping units represented in Formula I as each G¹ and G².

G¹ can be hydrogen. G¹ can be halogen. G¹ can be alkyne. G¹ can be optionally substituted aryl. G¹ can be optionally substituted heteroaryl. G¹ can be halogen. G¹ can be substituted aryl. G¹ can be silyl. G¹ can be diazonium salt. G¹ can be triflate. G¹ can be acetyloxy. G¹ can be azide. G¹ can be sulfonate. In some aspect, G¹ can be phosphate. G¹ can be boronic acid substituted aryl. G¹ can be boronic ester substituted aryl. G¹ can be boronic ester. G¹ can be boronic acid. G¹ can be optionally substituted tetrahydropyrene (THP). G¹ can be optionally substituted dihydrophenanthrene (DHP). G¹ can be optionally substituted fluorene. In some aspect, G¹ can be aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof. G¹ can optionally be conjugated to a substrate, binding agent, chromophore, acceptor dye, or functional tag. G¹ can optionally be conjugated to a substrate or binding agent.

In some aspect, G² can be hydrogen. G² can be halogen. G² can be alkyne. G² can be optionally substituted aryl. G² can be optionally substituted heteroaryl. G² can be halogen. G² can be substituted aryl. G² can be silyl. G² can be diazonium salt. G² can be triflate. G² can be acetyloxy. G² can be azide. G² can be sulfonate. G² can be phosphate. G² can be boronic acid substituted aryl. G² can be boronic ester substituted aryl. G² can be boronic ester. G² can be boronic acid. G² can be optionally substituted tetrahydropyrene (THP). G² can be optionally substituted fluorene. G² can be optionally substituted dihydrophenanthrene (DHP). G² can be aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof. G² can optionally be conjugated to a substrate, binding agent, chromophore, acceptor dye, or functional tag. G² can optionally be conjugated to a substrate or binding agent.

G¹ and G² can each independently be optionally substituted dihydrophenanthrene (DHP). G¹ and G² can each independently be optionally substituted fluorene. G¹ and G² can each independently be aryl substituted with one or more pendant chains terminated with a functional group. G¹ and G² can each independently be a heteroaryl substituted with one or more pendant chains terminated with a functional group.

G¹ and G² can each independently be

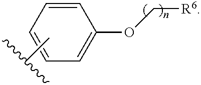

G¹ and G² can each independently be

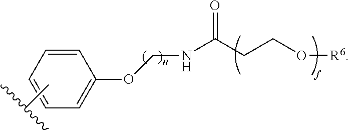

G¹ and G² can each independently be

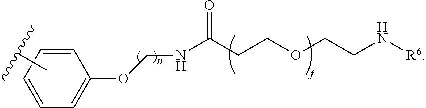

G¹ and G² can each independently be

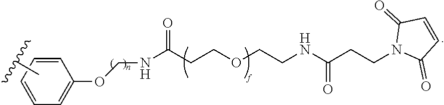

G¹ and G² can each independently be

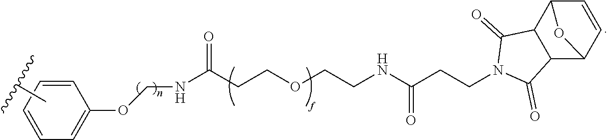

G¹ and G² can each independently be

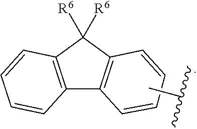

G¹ and G² can each independently be

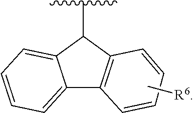

G¹ and G² can each independently be

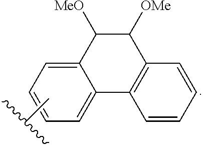

G¹ and G² can each independently be

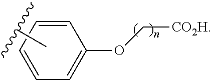

R⁶, f and n can be as described above.

In some embodiments, the water-soluble fluorescent polymer can be

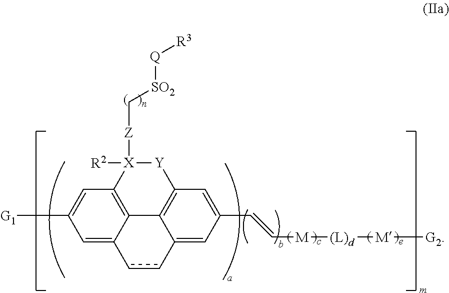

The water-soluble fluorescent polymer can be

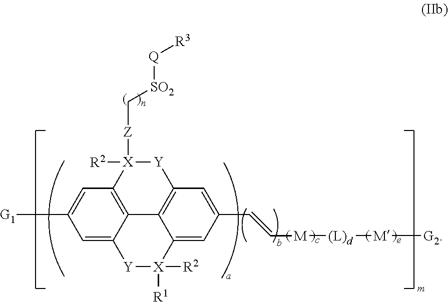

The water-soluble fluorescent polymer can be

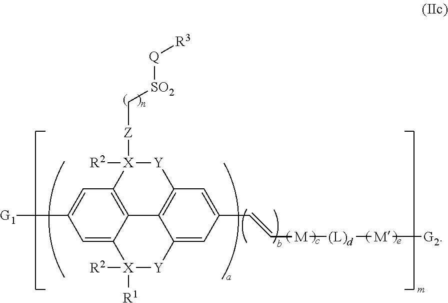

The water-soluble fluorescent polymer can be

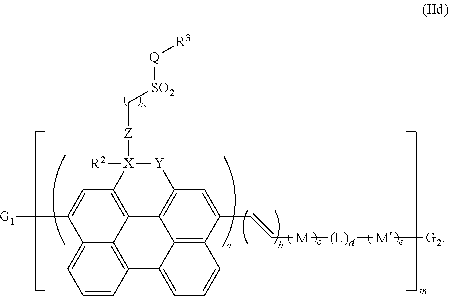

The water-soluble fluorescent polymer can be

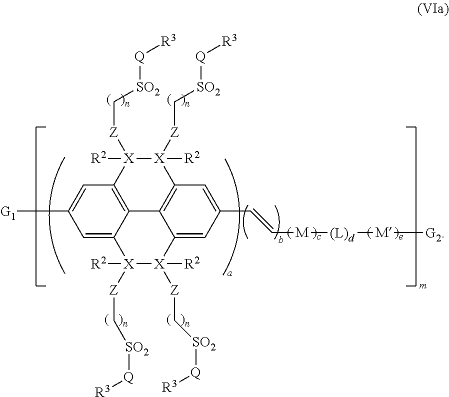

The water-soluble fluorescent polymer can be

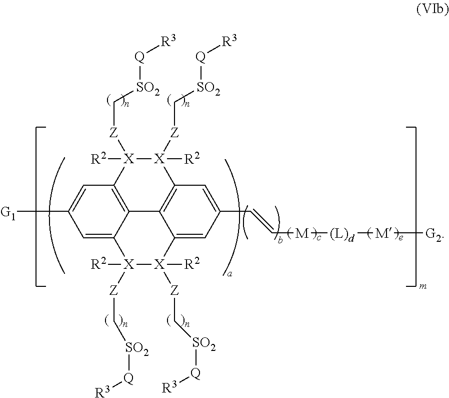

Each of X, Y, R¹, R², R³, Q, Z, R⁴, M, M′, R⁵, L, G¹, G², a, b, c, d, e, m, and n can be as described above.

In some embodiments, the water-soluble fluorescent polymer can be

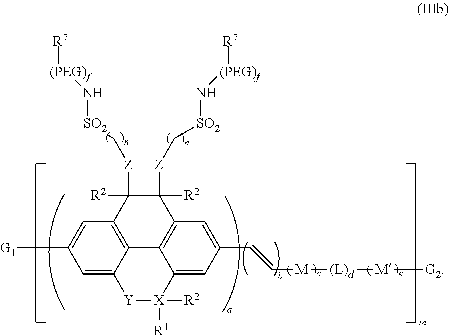

The water-soluble fluorescent polymer can be

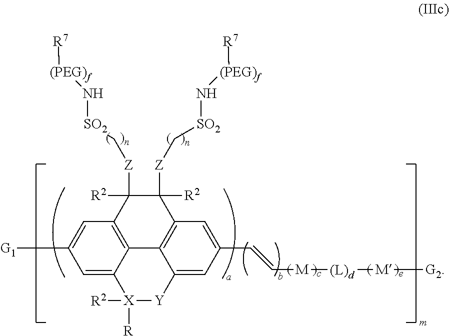

The water-soluble fluorescent polymer can be

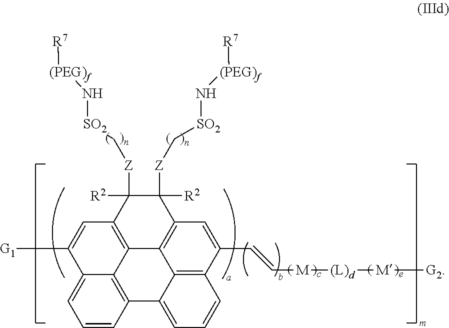

The water-soluble fluorescent polymer can be

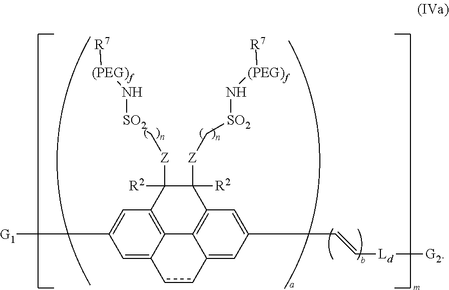

The water-soluble fluorescent polymer can be

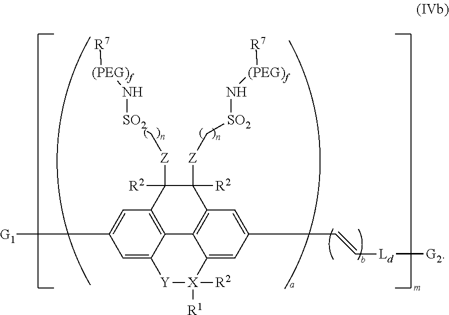

The water-soluble fluorescent polymer can be

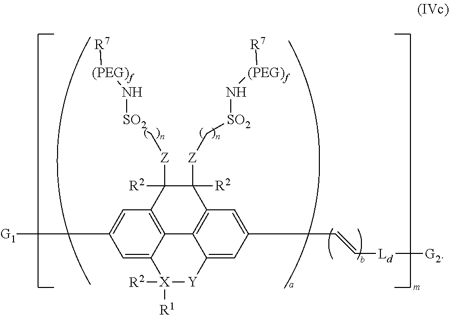

The water-soluble fluorescent polymer can be

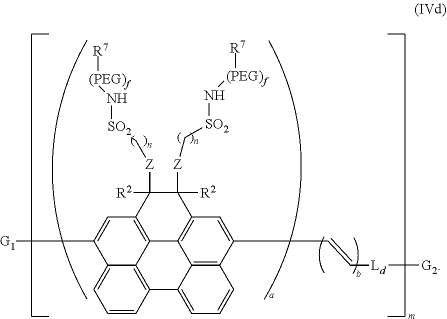

The water-soluable fluorescent polymer can be

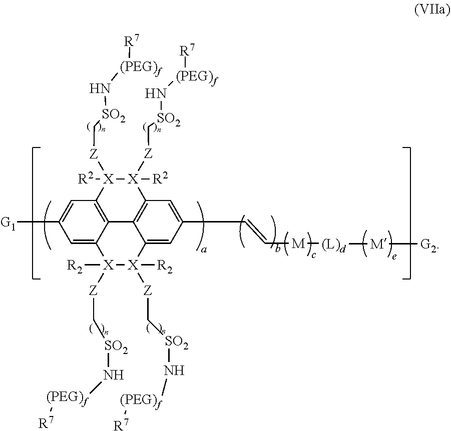

The water-soluble fluorescent polymer can be

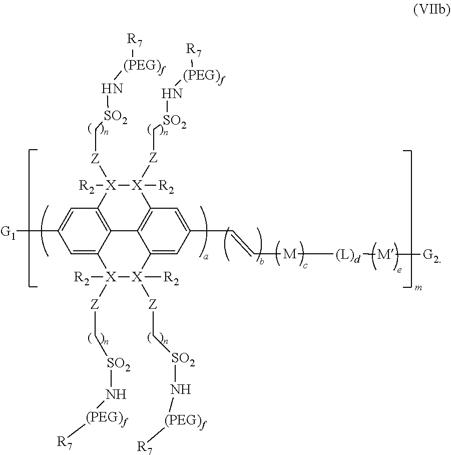

Each of X, Y, R¹, R², R³, Q Z, R⁴, M, M′, R⁵, L, G¹, G², a, b, c, d, e, m, and n can be as described above. R⁷ can be H. R⁷ can be C₁-C₁₂ alkyl. R⁷ can be C₂-C₁₂ alkene. R⁷ can be C₂-C₁₂ alkyne. R⁷ can be C₃-C₁₂ cycloalkyl. R⁷ can be C₁-C₁₂ haloalkyl. R⁷ can be C₁-C₁₂ alkoxy. R⁷ can be C₂-C₁₈ (hetero)aryloxy. R⁷ can be C₂-C₁₈ (hetero)arylamino. R⁷ can be C₂-C₁₂ carboxylic acid. R⁷ can be C₂-C₁₂ carboxylate ester. R⁷ can be C₁-C₁₂ alkoxy. f can independently be an integer from 0 to 50.

In some embodiments, the water-soluble fluorescent polymer can be

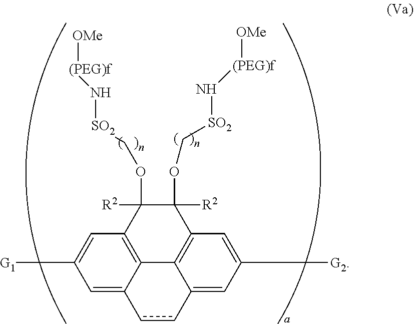

The water-soluble fluorescent polymer can be

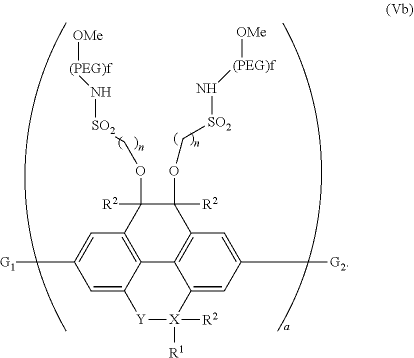

The water-soluble fluorescent polymer can be

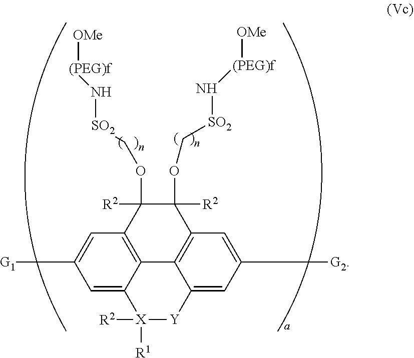

The water-soluble fluorescent polymer can be

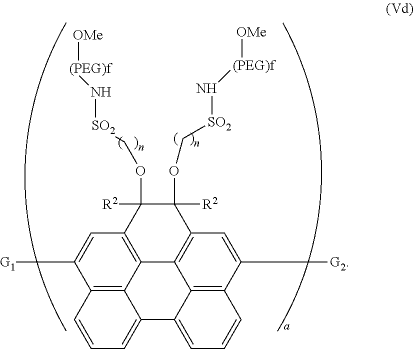

The water-soluble fluorescent polymer can be

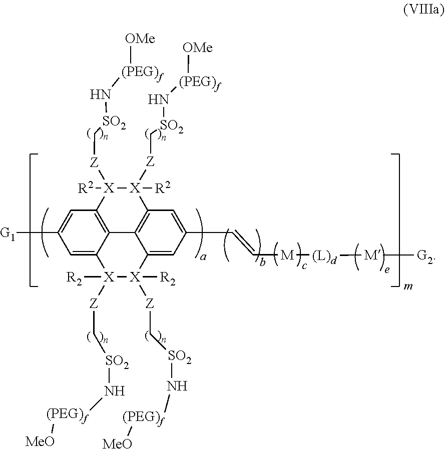

The water-soluble fluorescent polymer can be

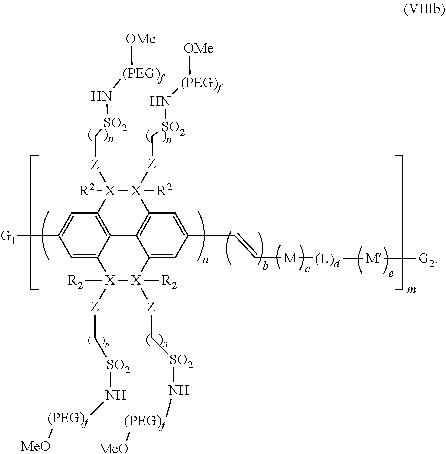

Each of X, Y, R¹, R², R³, Q Z, R⁴, M, M′, G¹, G², a, b, c, d, e, and n can be as described above; and f can independently be an integer from 0 to 50.

In some embodiments, the water-soluble fluorescent polymer of the invention can be a water-soluble fluorescent copolymer comprising a combination of polymers, wherein at least one or more of the water-soluble fluorescent polymers has a structure selected from the group consisting of Formula I, Formula (IIa), Formula II(b), Formula (IIc), Formula (IId), Formula (IIIa), Formula (IIIb), Formula (IIIc), (IIId), Formula (IVa), Formula (IVc), Formula-(IVd), Formula (Va), Formula (Vb), Formula (Vc), Formula (Vd), Formula (VIa), Formula (VIb), Formula (VIIa), Formula (VIIb), Formula (VIIIa), Formula (VIIIb). The copolymers can additionally comprise a co-monomer known in the art. The co-monomer can be a π-conjugated co-monomer. The π-conjugated co-monomer can be selected from the group consisting of optionally substituted fluorene monomers, optionally substituted dihydrophenanthrene (DHP) monomers, optionally substituted fluorenooxepine monomers, or optionally substituted benzene monomers. The co-monomer can be any monomer disclosed in WO 2017/180998A2 and U.S. Pat. Nos. 7,629,448, 8,158,444, 8,362,193, 8,575,303, 8,802,450, 8,969509, 9371559, or 9383353, the disclosures of which are incorporated herein in their entireties.

In some cases, the water-soluble fluorescent copolymer can be

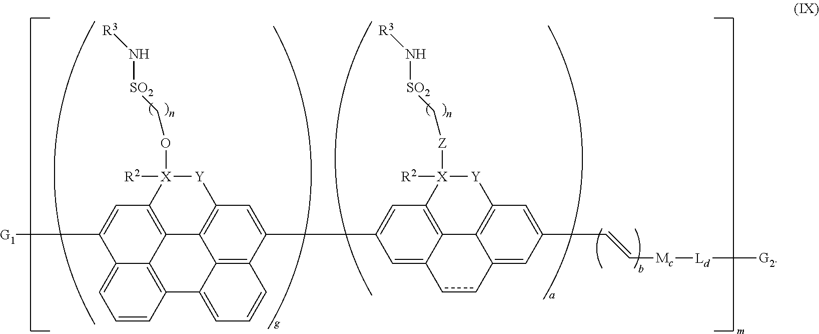

The water-soluable fluorescent copolymer can be

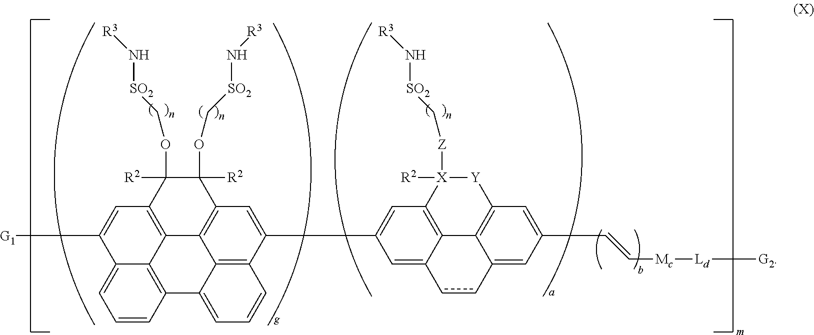

The water-soluable fluorescent copolymer can be

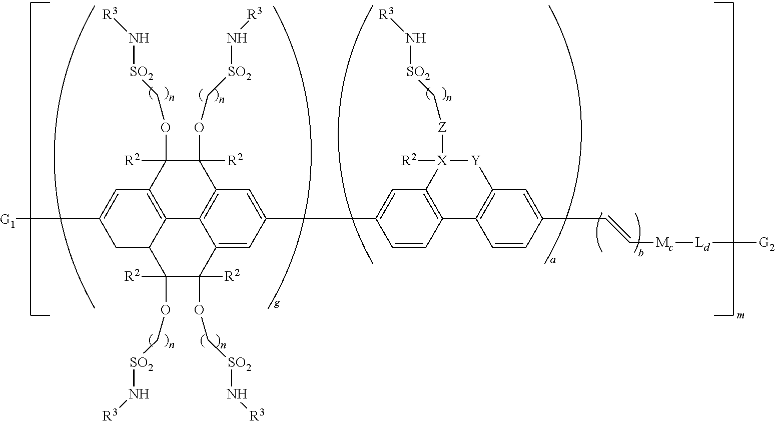

The water-soluable fluorescent copolymer can be

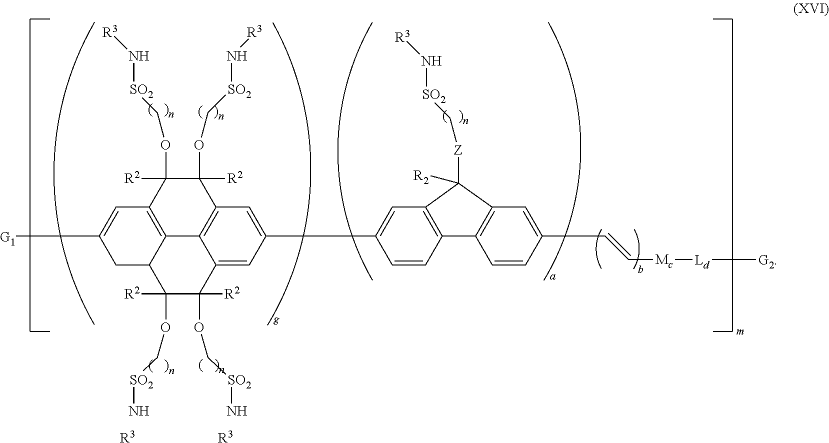

Each of X, Y, R¹, R², R³, Q Z, R⁴, M, R, L, G¹, G², a, b, c, d, m, and n are as described above.

In some cases, the water-soluble fluorescent copolymer can be

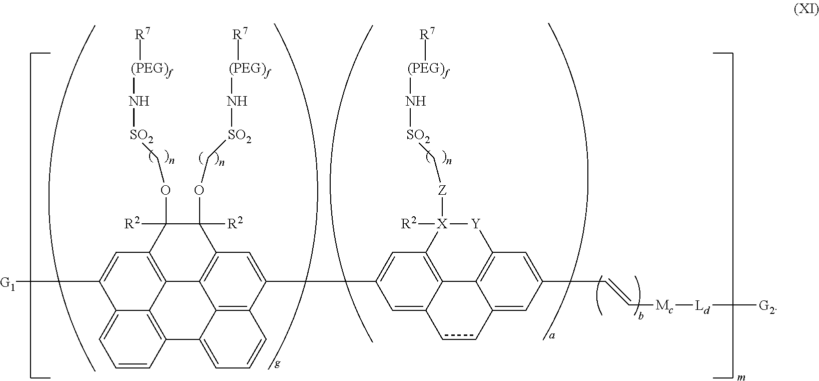

The water-soluble fluorescent copolymer can be

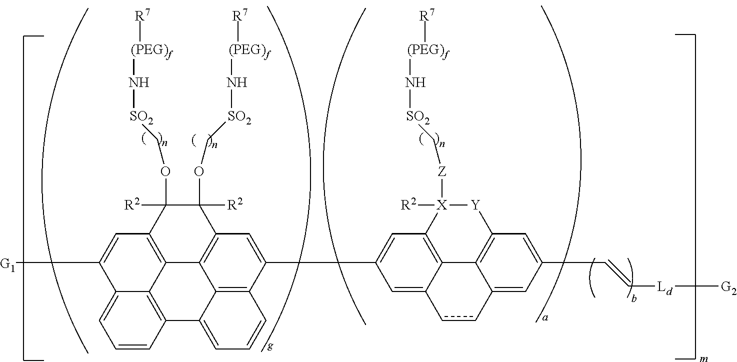

The water-soluble fluorescent copolymer can be

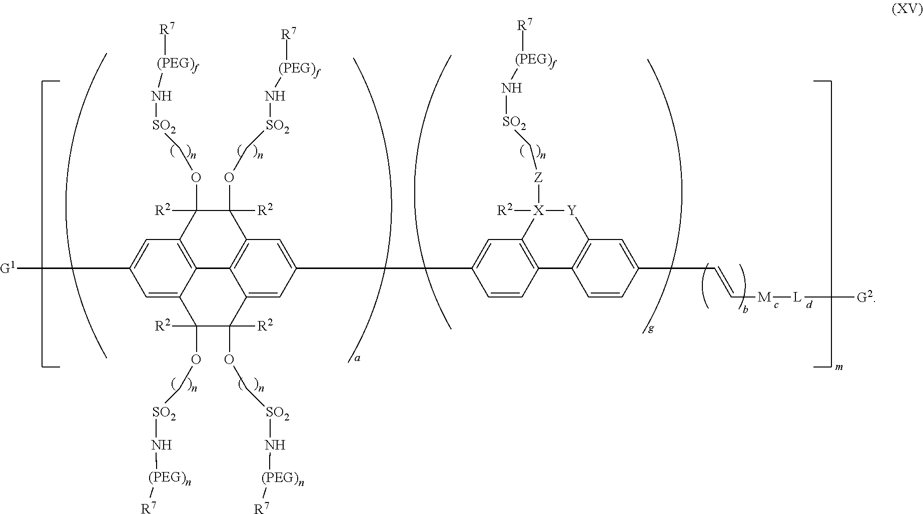

The water-soluble fluorescent copolymer can be

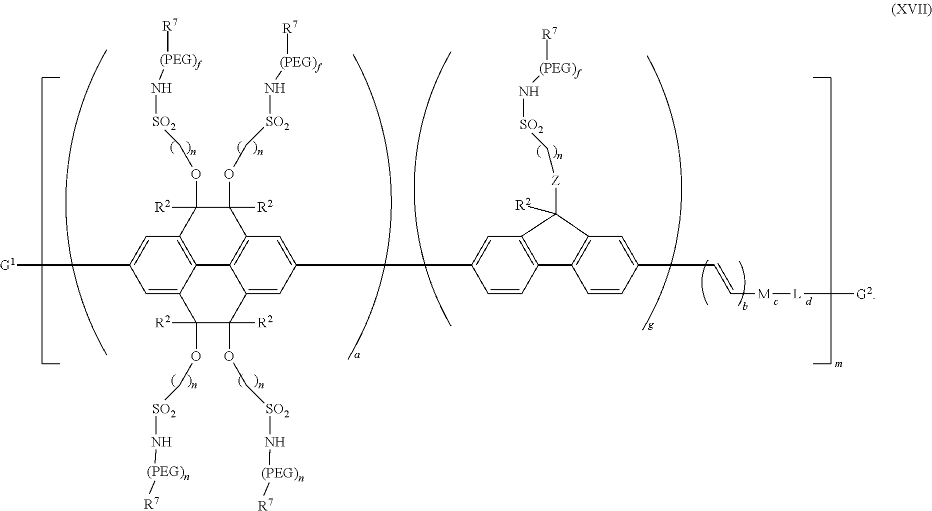

Each of X, Y, R¹, R², R³, Q, Z, R⁴, M, R⁵, L, G¹, G², a, b, c, d, m, and n can be as described above. R⁷ can be H. R⁷ can be C₁-C₁₂alkyl. R⁷ can be C₂-C₁₂alkene. R⁷ can be C₂-C₁₂ alkyne. R⁷ can be C₃-C₁₂cycloalkyl. R⁷ can be C₁-C₁₂ haloalkyl. R⁷ can be C₁-C₁₂ alkoxy. R⁷ can be C₂-C₁₈ (hetero)aryloxy. R⁷ can be C₂-C₁₈ (hetero)arylamino. R⁷ can be C₂-C₁₂ carboxylic acid; R⁷ can be C₂-C₁₂ carboxylate ester; R⁷ can be C₁-C₁₂ alkoxy. f can be independently an integer from 0 to 50.

In some cases, the water-soluble fluorescent copolymer can be

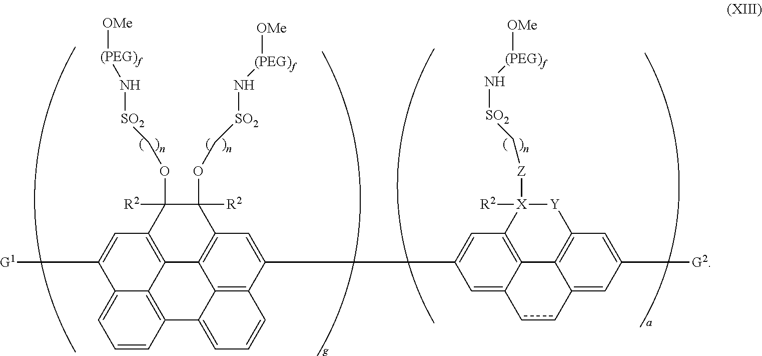

Each of X, Y, R¹, R², R³, Q, Z, R⁴, M, R⁵, L, G¹, G², a, b, c, d, m, and n can be as described above; and f can independently be an integer from 0 to 50.

### Monomers

Water-soluble monomers of the present disclosure include tetrahydropyrenes (THP). For example, water-soluble monomers of the present disclosure include:

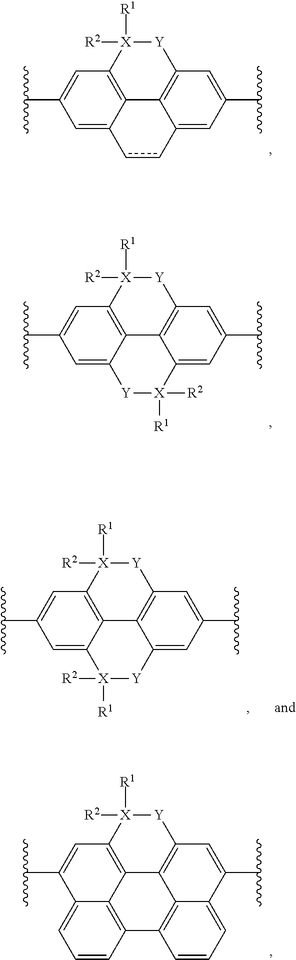

wherein
each of X, Y, R¹, R², R³, Q, Z, R⁴, M, R⁵, L, G¹, G², a, b, c, d, m, and n can be as described above. In some cases, R¹ is

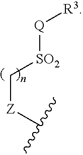

In some cases, R¹ is

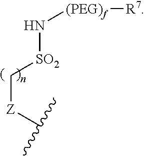

In some cases, R¹ is

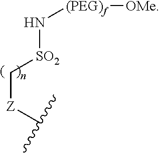

In some cases, Q is NH, R¹ is

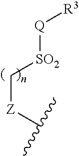

and R³ is PEG. In some cases, Q is NH, R¹ is

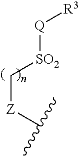

### and R¹ is

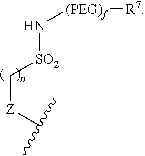

In some cases, Q is NH and R¹ is

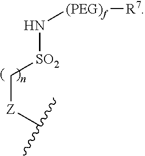

In some cases, Q is NH and R¹ is

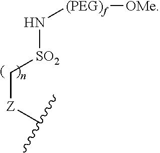

In some cases, X is C and R¹ is

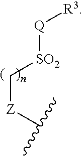

In some cases, X is C and R¹ is

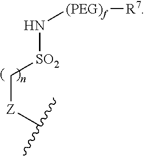

In some cases X is C and R¹ is

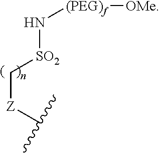

In some cases, X is C and R¹ is

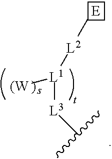

In some cases, X is C, Q is NH, R¹ is

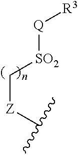

and R³ is PEG. In some cases, X is C, Q is NH, R¹ is

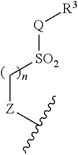

### and R³ is

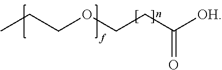

In some cases, X is C, Q is NH and R¹ is

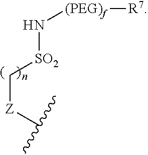

In some cases, X is C, Q is NH and R¹ is

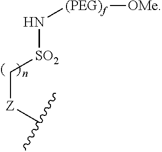

In some X is C and Y is CR¹R². In some cases, X is C, Y is CR¹R², and R¹ is

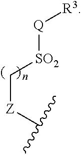

In some cases, X is C, Y is CR¹R², and R¹ is

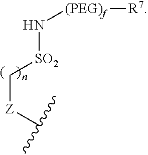

In some cases, X is C, Y is CR¹R², and R¹ is

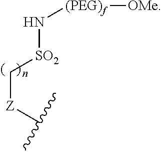

In some cases, X is C, Y is CR¹R², and R¹ is

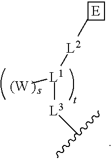

In some cases, X is C, Y is CR¹R², Q is NH, R¹ is

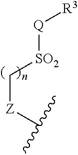

and R³ is PEG. In some cases, X is C, Y is CR¹R², Q is NH, R¹ is

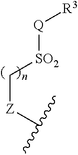

### and R³ is

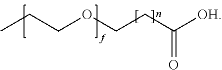

In some cases, X is C, Y is CR¹R², Q is NH and R¹ is

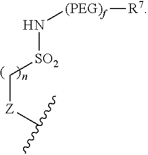

In some cases, is X is C, Y is CR¹R², Q is NH and R¹ is

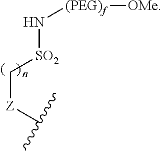

In some embodiments, water-soluble monomers of the present disclosure include:

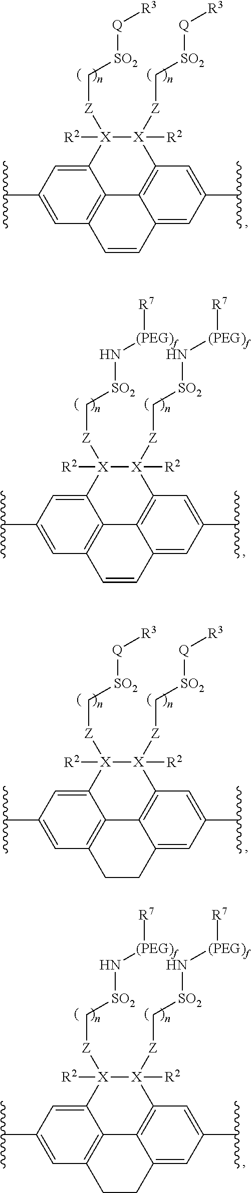

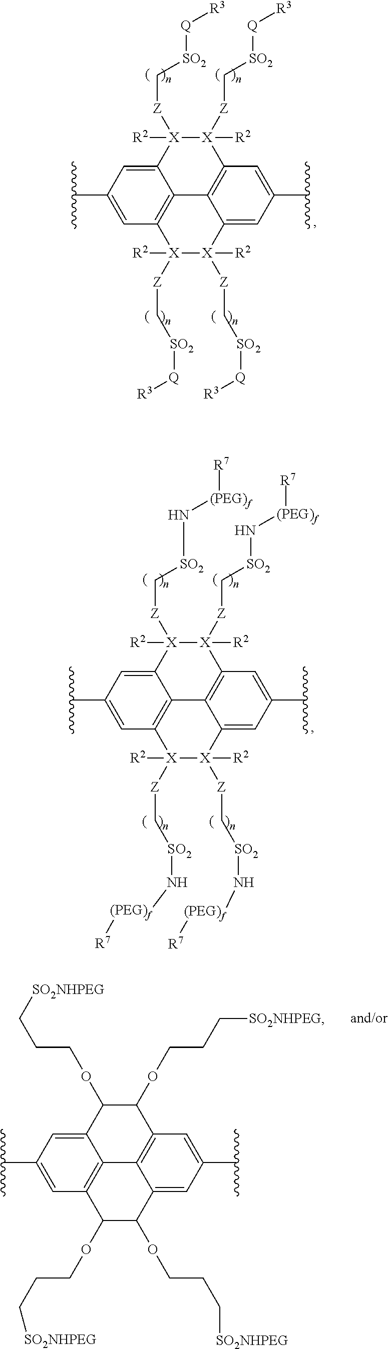

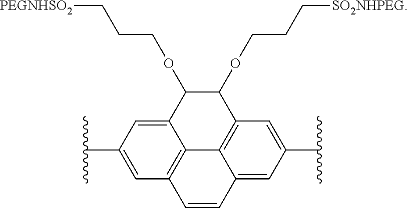

Both terminal ends of the water-soluble monomers of the present invention are independently or both a halogen atom, boronic ester or boronic acid, silyl, diazonium salt, triflate, acetyloxy, sulfonate, or phosphate which can undergo Pd or Nickel salt catalyzed polymerization reactions.

### Synthesis

Water-soluble THP monomers of the present disclosure can be made as shown below.

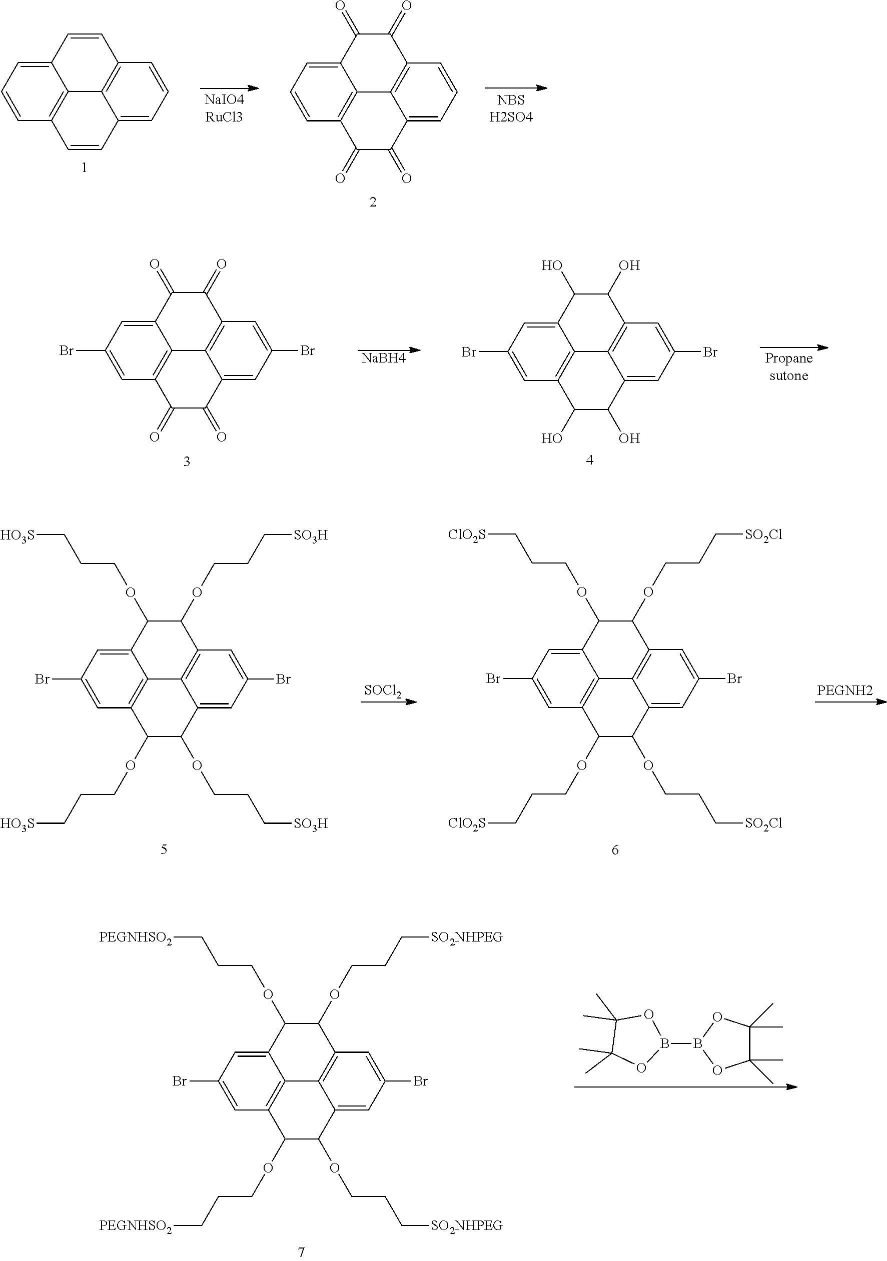

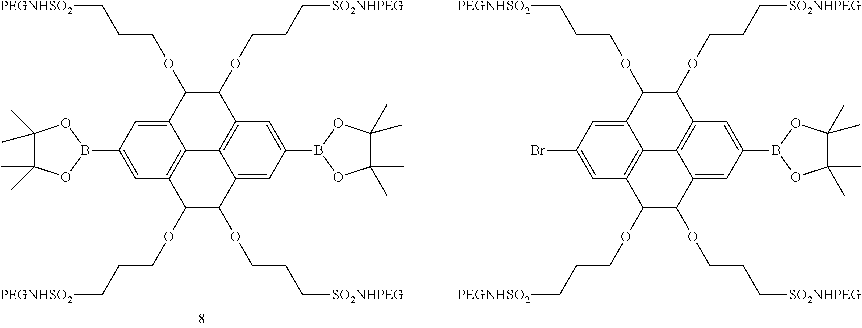

For example, Compound 2 can be prepared as follows: In a conical flask, mix pyrene with dichloromethane, acetonitrile and water for 5 minutes. To this, add NaIO₄ and RuCl₃ and heat the reaction mixture to 40° C. for 16 hours. Wash the crude mixture with water, to obtain pure product Compound 2.

Compound 3 can be made as follows: Add NBS to Compound 2 in sulfuric acid and keep the mixture at 40° C. for overnight. Compound 3 precipitates from water. Filter the precipitated product Compound 3.

Compound 4 can be prepared as follows: In a conical flask, add NaBH₄ into a stirring water-ethanol mixture. To this solution, add Compound 3 portion-wise but quickly (within 5 min). Allow the reaction mixture to stir for a day. Stop the reaction and neutralize the reaction mixture with diluted HCl acid. After the neutralization, filter the white precipitate and wash with excess water. Wash the white precipitate with very cold (<−15° C.) ethanol and methanol to obtain Compound 4.

Compound 5 can be prepared as follows: In a 2 neck round bottom flask, dissolve THP-OH (Compound 4) and 18C6 in THF. Purge the solution with nitrogen for 20 minutes and add while nitrogen purging continues. In another RB, dissolve 1,3 propane sultone in THF and purge with nitrogen. Add this sultone solution to DHP-OH solution by addition funnel over a period of 20-30 minutes. Stir the reaction at RT for 4-5 hrs. Evaporate the solvents and dissolve the precipitate in water. Add acetone to obtain white precipitate in the form of disodium salt. Filter the precipitate and re-dissolve in water (minimal amount), neutralize with HCl, and precipitate again in acetone. Compound 5 is obtained after repeated precipitation (2-3 times) followed by centrifugation.

Compound 6 can be prepared as follows: In a round bottom flask, mix Compound 5 with DMF. To this, add SOCl₂ dropwise and allow the mixture allowed to overnight. The next morning, pour the reaction mixture into water and filter and dry the precipitate to obtain Compound 6.

Compound 7 can be prepared as follows: Mix Compound 6 with 2.2 equivalent of PEG amine in a dichloromethane/TEA mixture. After 3 hours, sonicate the reaction and extract the crude product in dichloromethane followed by column chromatography (silica gel, MeOH—CHCl₃) to obtain Compound 7.

Compound 8 can be prepared as follows: Mix Compound 7 with DMSO under nitrogen. To this mixture, add 3 equivalents of bispinacolatodiboron. React the reagents with 12 equivalent of potassium acetate and 4 equivalents of Pd(dppf)Cl₂ catalyst for 5 hours at 80° C. Cool down the reaction mixture and extract with CHCl₃/water. Concentrate the organic layer and purify by column chromatography (silica gel, MeOH—CHCl₃) to obtain Compound 8.

### Polymerization

The compounds described in the above embodiments may be made using procedures known in the art. In some embodiments, water-soluble fluorescent polymers can be made from water-soluble tetrahydropyrene (THP) monomers combined with electron rich linker units. In some embodiments, bright water-soluble fluorescent polymeric dyes can be made from a combination of THP with any one or more of optionally substituted dihydrophenanthrene (DHP) monomers, optionally substituted fluorene monomers, and/or optionally substituted benzene monomers combined with electron rich linker units. Fluorene and DHP monomers and methods for making them are disclosed in WO 2017/180998. Optionally substituted benzene monomers are known and commercially available, for example, from Sigma Aldrich.

Generally, polymerization of monomer units described above can be accomplished using polymerization techniques known to those of skill in the art or using methods known in the art in combination with methods described herein. For example, synthesis of diboronic ester derivatives from a dihalide monomer can be accomplished via Suzuki coupling with bis(pinacolato) diboron:

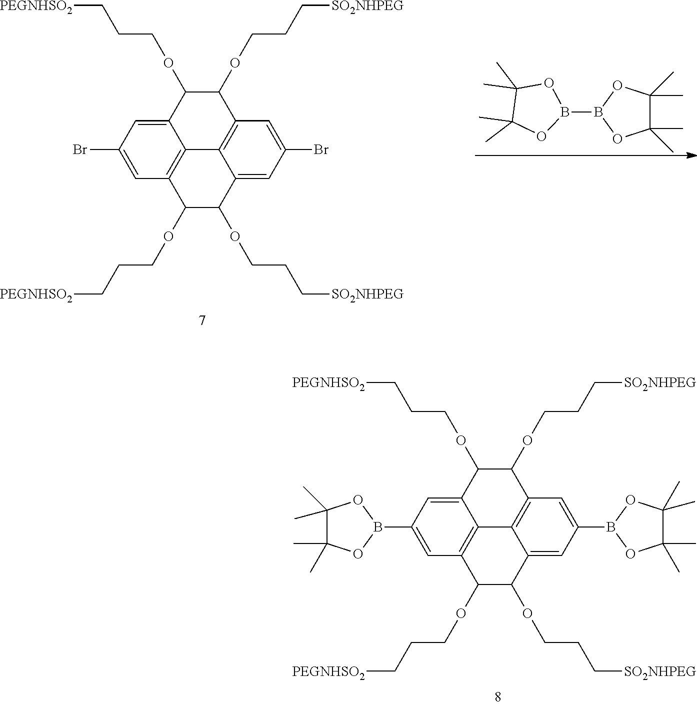

Similarly, polymerization can also be achieved via Suzuki coupling:

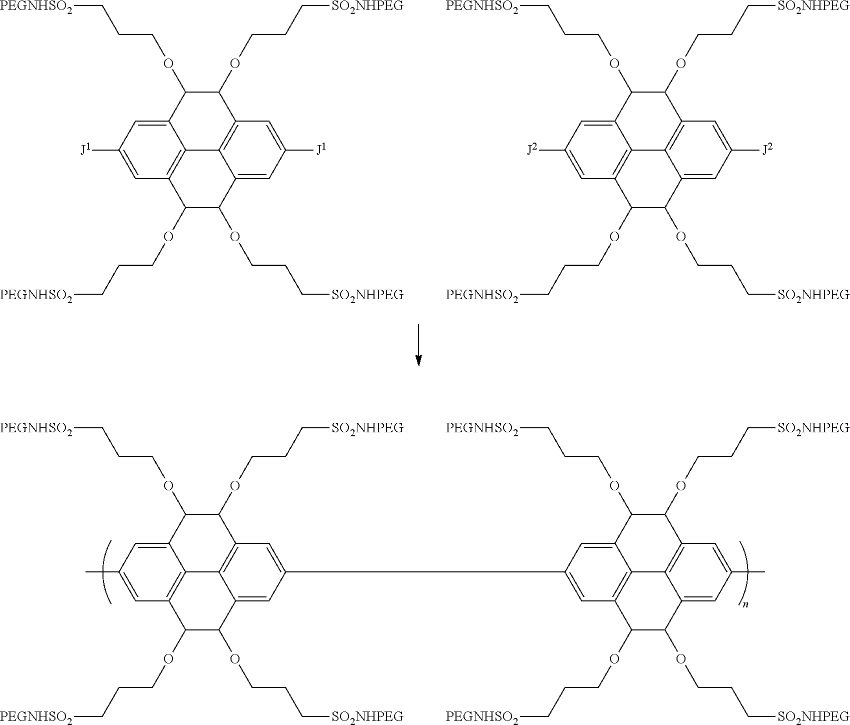

where J¹ and J² are independently H, Br, B(OH)₂, or a boronic ester.

For example, polymerization can proceed as follows. In a round bottom flask, add both the bromo and boronic monomers in (DMF-water) mixture and purge with nitrogen for 10 minutes. Under nitrogen, mix about 20 equivalent of CsF and 10% of Pd(OAc)₂ and heat at 80° C. Monitor polymerization using UV-Vis spectroscopy and SEC chromatography. Later, add a capping agent (selected from G¹) containing an appropriate functional group to the reaction mixture and 3 hours later add a second capping agent (selected from G²) to the reaction mixture. After the reaction is complete, evaporate off the crude reaction mixture and pass it through a gel filtration column to remove small organic molecules and low MW oligomers.

### Capping Units

Linkers and capping units can be conjugated to a water-soluble fluorescent polymer backbone of this disclosure via similar mechanisms as described previously. For example, bromo- and boronic esters of capping units can be used to append one or both ends of a polymer. Utilizing both bromo- and boronic esters of capping units will append both ends of polymer. Utilizing only one form, either a bromo- or boronic ester of a capping unit, will append only those ends terminated with its respective complement and for symmetric polymerizations can be used to statistically modify only one end of a polymer. For asymmetric polymers this approach is used to chemically ensure the polymers are only modified at a single chain terminus. Capping units can also be appended asymmetrically by first reacting a bromo-capping unit with a polymer with Y ends and subsequently reacting the polymer with a boronic ester capping unit.

For example, capping agents of the present disclosure can be made as shown below.

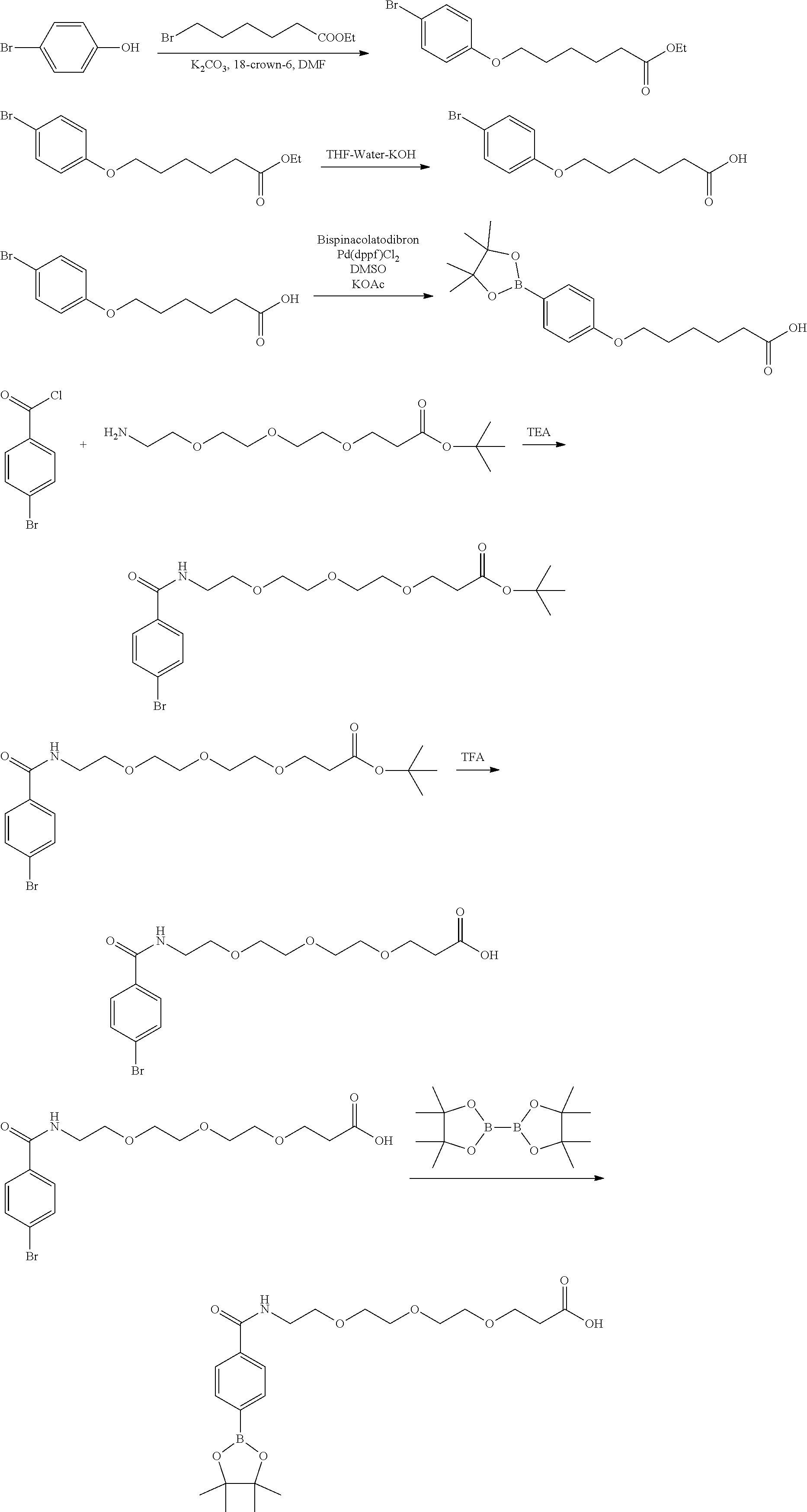

### Binding Agents

A “binding agent” of the invention can be any molecule or complex of molecules capable of specifically binding to target analyte. A binding agent of the invention includes, for example, proteins, small organic molecules, carbohydrates (including polysaccharides), oligonucleotides, polynucleotides, lipids, affinity ligand, antibody, antibody fragment, an aptamer and the like. The antibody fragment can be a target analyte-binding antibody fragment. In some embodiments, the binding agent is an antibody or a fragment thereof. Specific binding in the context of the present disclosure refers to a binding reaction which is determinative of the presence of a target analyte in the presence of a heterogeneous population. Thus, under designated assay conditions, the specified binding agents bind preferentially to a particular protein or isoform of the particular protein and do not bind in a significant amount to other proteins or other isoforms present in the sample.

When the binding agents are antibodies, they may be monoclonal or polyclonal antibodies. The term antibody as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and a Fab expression library.

### Complexes

In general, water-soluble fluorescent polymers of the present disclosure can be conjugated to binding agents to form a conjugated water-soluble fluorescent polymer complex using techniques known to those of skill in the art or using methods known in the art in combination with methods described herein.

In some embodiments, water-soluble fluorescent polymers of the invention can be conjugated to binding agents using the method of direct modification of core polymers described in US2020/0190253, which is incorporated herein by reference in its entirety.

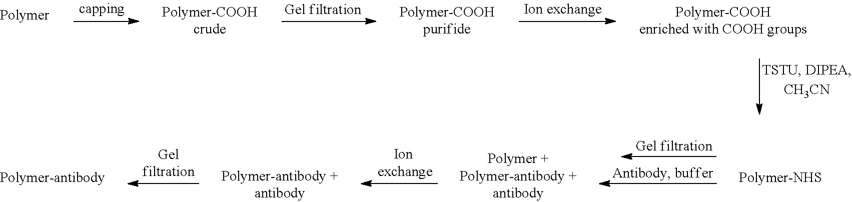

For example, preparation of polymer NHS ester can proceed as follows. Using a clean vial, dissolve 5 mg of polymer in 1 mL dry CH₃CN. To this, add 15 mg N,N,N′,N′—tetramethyl-O—(N-succinimidyl)uranium tetrafluoroborate (TSTU) and stir for 2 more minutes. To this, add 100 uL N,N-diisopropylethylamine (DIPEA) and continue stirring overnight with the cap sealed with parafilm. Later evaporate off the organic solvents in the reaction mixture. Dissolve the crude NHS in about 750 μL of 1×BBS buffer (pH 8.8) by a quick vortex and transfer it to a Zeba column 40K MWCO. Spin down the sample at 2200 RPM for 2 min and use this polymer NHS immediately.

Conjugation of polymer NHS with CD4 can proceed as follows. Take the polymer NHS in 1×BBS (˜800 uL), spin down, add to 0.6 mg of CD4 and mix with 100 μL of 0.5M Borate buffer (pH 9.0). Vortex quickly for 30 seconds and allow to mix for 3-4 hours in the coulter mix.

Purification of polymer-antibody conjugate through Histrap HP column can proceed as follows. Approach 1: After the crude reaction purify the conjugate using a Histrap HP column. Load the sample using 1×PBS buffer and collect the unbound fraction. This can be done using 20 CV of buffer. Later change the buffer to wash the bound fraction which has both conjugate and free antibody. This can be done using 1×PBS with 0.25M imidazole running for 10 CV.

Approach 2: Histrap SP Sepharose FF column. Equilibrate the column and load the sample using 20 mM Citrate buffer pH 3.5 and collect the unbound fraction. This can be done using 20 CV of buffer. Later change the buffer to elute the bound fraction which has both conjugate and free antibody. This can be done using 20 mM Tris buffer pH 8.5 running for 20 CV.

Approach 3: Load the crude conjugate in a Tangential flow filtration system equipped with a 300K MWCO membrane. The conjugate is washed using 1×PBS until the filtrate show no absorption at 405 nm. Later the compound is concentrated.

Purification of polymer-antibody conjugate through SEC column can proceed as follows. Load the crude conjugate containing free antibody to the Size Exclusion Column, using 1×PBS. Pool the tubes after checking the absorption spectra and concentrate in an Amicon Ultra-15 having a 30 KDa MWCO centrifugal concentrator.

Purification of polymer-antibody conjugate through a Nuvia HR-S column (Bio-Rad Laboratories, Inc.) can proceed as follows. Load crude polymer-antibody conjugate mixture to the Nuvia HR-S column using a biological buffer having a pH between about 2 to about 14 and a conductivity less than 3 mS/cm. Due to charge-charge interactions between the matrix and biomolecules, the polymer antibody conjugate will bind to the resin while free polymer dye will not interact with the resin and will flow through. Disrupt the charge-charge interactions between the matrix and polymer antibody conjugate by using a salt (e.g., NaCl, KCl, phosphate etc.) at a concentration ranging from about 0.1 to 2 M. The salt concentration can be reduced by adjusting the pH of the elution buffer. For example, the conjugate can be eluted using a biological buffer and gradient of salt concentration (e.g., NaCl, KCl) between about 100 to 1000 mM at a pH of between about 6 to about 10.

Purification of conjugate through a Nuvia cPrime column (Bio-Rad Laboratories, Inc.) can proceed as follows. Load crude polymer-antibody conjugate mixture to the Nuvia cPrime column using a biological buffer having a pH between about 2 to about 14 and a salt concentration (e.g., NaCl, KCl) ranging from 0 to about 1 M. Unreacted polymers will flow through the column while the polymer-antibody conjugate will bind to the column. Elute the polymer-antibody conjugate by increasing the pH of the elution buffer. For example, the crude polymer antibody conjugate can be loaded into the Nuvia cPrime column using a biological buffer pH 5.0, 5 mM NaCl and eluted with a biological buffer pH 7.0 and gradient of salt concentration 5 to 500 mM.

Purification of conjugate through an Anti-mouse anti-H+L antibody-agarose bead can proceed as follows. Mix crude polymer-antibody conjugate mixture with anti-mouse anti-H+L antibody-agarose bead in a biological buffer having a pH between about 6 to about 8 for about 30 minutes at room temperature. The anti-mouse anti H+L antibody-agarose bead will bind to the polymer antibody conjugate. Remove unreacted polymers by washing with the above-mentioned biological buffer using a benchtop centrifuge with a speed of 300 g for 3 minutes. Repeat the washing process at least three times. To elute the polymer-antibody conjugate, apply an IgG elution buffer with a pH ranging from about 2 to about 4 to the washed antibody-agarose bead and incubate for about 10 to 15 min. Centrifuge to collect the flow through that contains the polymer antibody conjugate.

### Tandem Polymers

In some embodiments, the fluorescent polymers or water-soluble fluorescent polymers of the disclosure, and conjugates thereof, include acceptor dyes or chromophores attached to the backbone. When a light source excites the polymer backbone, the acceptor dyes or chromophores can absorb energy of an appropriate wavelength and emit or transfer energy. Acceptor dyes useful in the disclosure include, for example, a cyanine dye, a xanthene dye, a coumarin dye, a thiazine dye, an acridine dye, FITC, CY3B, Cy55, Alexa 488, Texas red, Cy5, Cy7, Alexa 750, Cy55, Cy3B, Cy3.5, Alexa 750, 800 CW, Biotium CF 555, diethyl coumarin, DY705 (Dyomics), DY431, DY485XL, DY500XL, DY610, DY640, DY654, DY 682, DY 700, DY 701, DY 704, DY 730, DY 731, DY732, DY 734, DY 752, DY 778, DY 782, DY 800, DY 831, 800CW, and the like. The acceptor dye may be a pendant acceptor dye. The tandem dye may be a THP containing polymer according to the present disclosure comprising one or more, two or more, three or more, 1-30, 2-20, or 2.5-10 acceptor dye moieties.

For example, of an acceptor dye attached to the water-soluble fluorescent polymer or water-soluble fluorescent polymer complex backbone can be as follows:

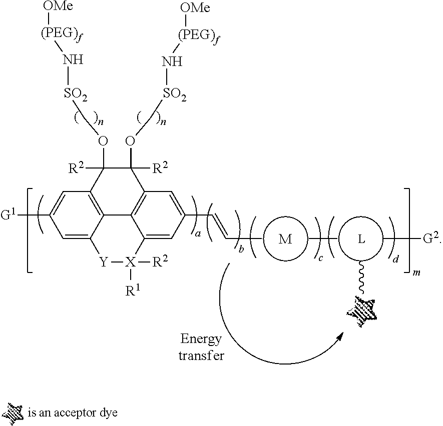

In another example, an acceptor dye attached to a water-soluble fluorescent polymer or water-soluble fluorescent polymer complex backbone can be as follows:

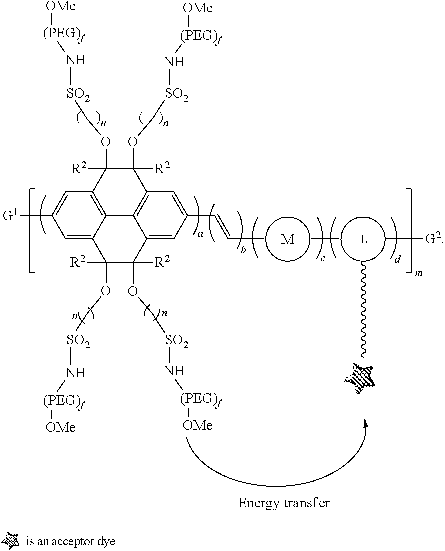

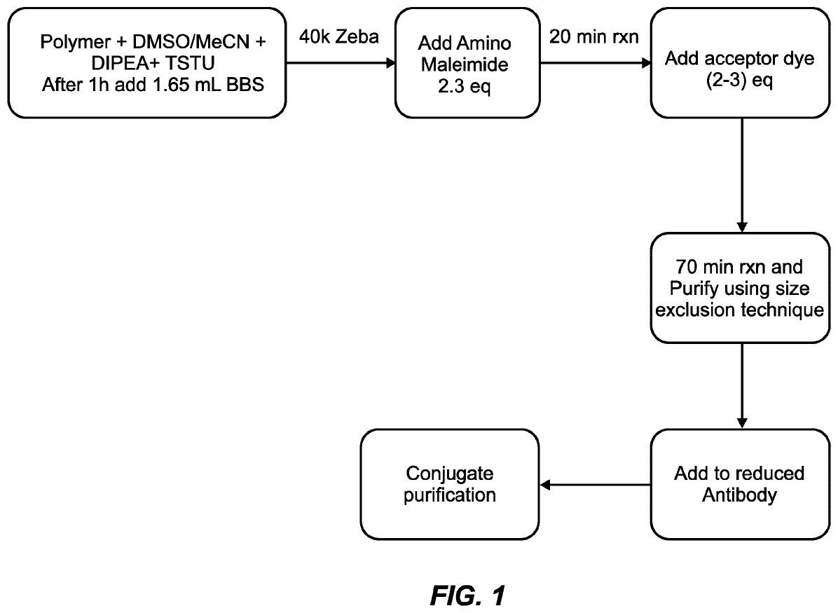

Water-soluble fluorescent tandem polymers or water-soluble fluorescent tandem polymer complexes can be prepared using techniques known to those of skill in the art or using methods known in the art in combination with methods described herein, including as shown in FIG. 1.

In some embodiments, instead of being attached to a Linker “L” in the polymer backbone, acceptor dyes, chromophores, fluorophores, functional moieties, functional tags, and binding agents can be attached to polymers of the invention through a linker moiety side group using the method of direct modification of core polymers described in US2020/0190253, which is incorporated herein by reference in its entirety. For example, the polymer may comprise an acceptor dye covalently attached via side-chain linker moieties L³-L¹ (e.g., when R⁴ is L²-E) to a THP monomer unit, as illustrated in any one of formula (Ia), (Ib), (Ic), (Id):

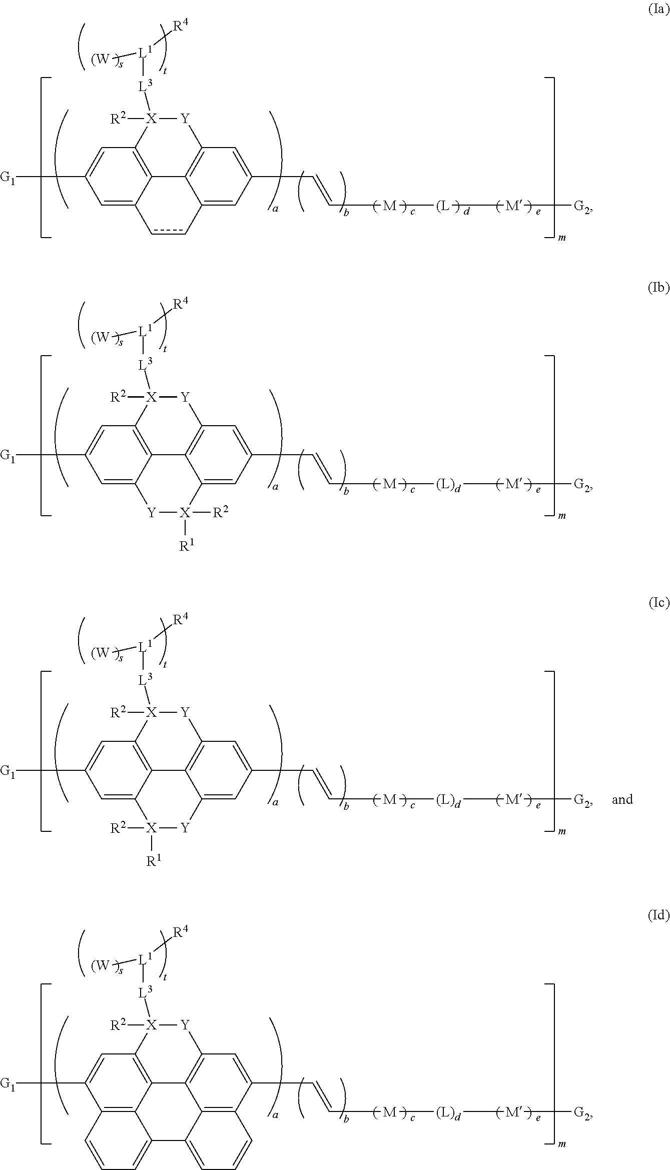

wherein
R⁴ is defined herein above, and at least one, 1-20, 2-15, or 3-12 of R⁴ is an acceptor dye, or linked acceptor dye.

### IV. Methods of Detecting an Analyte

### Overview

The present invention provides a method for detecting an analyte in a sample comprising:
providing a sample that is suspected of containing an analyte; and
contacting the sample with a binding agent conjugated to a water-soluble fluorescent polymer of the invention, wherein the binding agent is capable of interacting with the analyte.

A light source is applied to the sample that can excite the water-soluble fluorescent polymer; and light emitted from the conjugated water-soluble fluorescent polymer complex is detected. In the typical assay, water-soluble fluorescent polymers of the invention are excitable with a light having wavelength between about 395 nm and about 415 nm and the emitted light is typically between about 415 nm and about 475 nm. Alternatively, excitation light can have a wavelength between about 340 nm and about 370 nm and the emitted light can have a wavelength between about 390 nm and about 420 nm. While the water-soluble fluorescent polymers of the invention have a violet excitation spectrum, one of skill in the art will understand that the spectrum can be tuned to the blue, UV or another laser if the polymers are copolymerized with appropriate modifying units.

In the method of the invention, the water-soluble fluorescent polymer can be any-water-soluble fluorescent polymer of the invention as disclosed herein. The water-soluble fluorescent polymer can have the structure of Formula (I). The water-soluble fluorescent polymer can have the structure of any one or more of Formulas (IIa), (IIb), (IIc), (IId), (IIIa), (IIIb), (IIIc), (IIId), (IVa), (IVb), (IVc), (IVd), (Va), (Vb), (Vc), (Vd), (VIa), (VIb), (VIIa), (VIIb), (VIIIa), and (VIIIb).

In the method of the invention, the water-soluble fluorescent polymer can be a water-soluble fluorescent copolymer having the structure of any one of Formulas (IX), (X), (XI), (XII), (XIII), (XIV), (XV), (XVI) and (XVII) disclosed herein. The water-soluble fluorescent polymer can also be a water-soluble fluorescent copolymer comprising (1) at least one water-soluble fluorescent polymer having a structure selected from the group comprising of Formula (I), (IIa), (IIb), (IIc), (IId), (IIIa), (IIIb), (IIIc), (IIId), (IVa), (IVb), (IVc), (IVd), (Va), (Vb), (Vc), (Vd), (VIa), (VIb), (VIIa), (VIIb), (VIIIa), and (VIIIb) as disclosed herein; and (2) a π-conjugated co-monomer. The π-substituted comonomer can be an optionally substituted fluorene monomer. The π-substituted comonomer can be an optionally substituted dihydrophenanthrene (DHP) monomer. The π-substituted comonomer can be an optionally substituted benzene monomer. The optionally substituted fluorene and DHP co-monomer structures can be those disclosed in WO 2017/180998A2.

### Sample

The sample in the methods of the present disclosure can be, for example, blood, bone marrow, spleen cells, lymph cells, bone marrow aspirates (or any cells obtained from bone marrow), urine (lavage), serum, saliva, cerebral spinal fluid, urine, amniotic fluid, interstitial fluid, feces, mucus, or tissue (e.g., tumor samples, disaggregated tissue, disaggregated solid tumor). In certain embodiments, the sample is a blood sample. In some embodiments, the blood sample is whole blood. The whole blood can be obtained from the subject using standard clinical procedures. In some embodiments, the sample is a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). In some embodiments, the sample can be from a cell culture.

The subject can be a human (e.g., a patient suffering from a disease), a commercially significant mammal, including, for example, a monkey, cow, or horse. Samples can also be obtained from household pets, including, for example, a dog or cat. In some embodiments, the subject is a laboratory animal used as an animal model of disease or for drug screening, for example, a mouse, a rat, a rabbit, or guinea pig.

### Analytes

An “analyte” as used herein, refers to a substance, e.g., a molecule, whose abundance/concentration is determined by some analytical procedure. For example, in the present disclosure, an analyte can be a protein, peptide, nucleic acid, lipid, carbohydrate small molecule, or a target-associated biomolecule.

The target analyte may be, for example, nucleic acids (DNA, RNA, mRNA, tRNA, or rRNA), peptides, polypeptides, proteins, lipids, ions, monosaccharides, oligosaccharides, polysaccharides, lipoproteins, glycoproteins, glycolipids, or fragments thereof. In some embodiments, the target analyte is a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels and the like. The protein can be an activatable protein or a protein differentially expressed or activated in diseased or aberrant cells, including but not limited to transcription factors, DNA and/or RNA-binding and modifying proteins, nuclear import and export receptors, regulators of apoptosis or survival and the like.

### Assays

Assay systems utilizing a binding agent and a fluorescent label to quantify bound molecules are well known. Examples of such systems include flow cytometers, scanning cytometers, imaging cytometers, fluorescence microscopes, and confocal fluorescent microscopes.

In some embodiments, flow cytometry is used to detect fluorescence. A number of devices suitable for this use are available and known to those skilled in the art. Examples include BCI Navios, Gallios, Aquios, and CytoFLEX flow cytometers.

In other embodiments, an assay is used. The assay can be an immunoassay. Examples of immunoassays useful in the disclosure include, but are not limited to, fluoroluminescence assay (FLA), and the like. The assays can also be carried out on protein arrays.

When the binding agents are antibodies, antibody or multiple antibody sandwich assays can also be used. A sandwich assay refers to the use of successive recognition events to build up layers of various binding agents and reporting elements to signal the presence of a particular analyte. Examples of sandwich assays are disclosed in U.S. Pat. No. 4,486,530 and in the references noted therein.

### V. Examples

### Example 1: Preparation of Water-Soluble Fluorescent THP Polymer

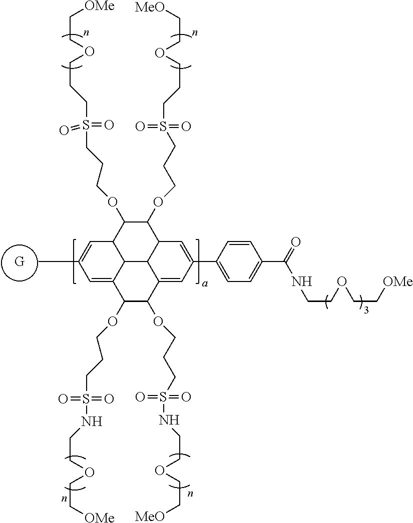

Method 1: In a round bottom flask add both a dibromo THP and a diboronic THP monomer (1:1) in a DMF-water mixture and purge the mixture with nitrogen for 10 minutes. Mix about 20 equivalent of CsF and 10% of Pd(OAc)₂ under nitrogen and heat at 80° C. Monitor polymerization using UV-Vis spectroscopy and SEC chromatography. Later, add a capping agent (selected from G¹) containing an appropriate functional group to the reaction mixture. Three hours later, add a second capping agent (selected from G²) to the reaction mixture. After the reaction is complete, evaporate off the crude reaction mixture and pass it through a gel filtration column to remove small organic molecules and low MW oligomers. Later, pass the crude polymer through a Tangential flow filtration system equipped with a 100K MWCO membrane, and wash using 20% ethanol until the absorption of the filtrate diminishes.

Method 2: Alternatively, the polymerization can be accomplished by self-polymerizing a bromo-boronic ester of a THP molecule. In a round bottom flask add THP bromoboronic ester to a DMF-water mixture and purge with nitrogen for 10 minutes. Mix about 10 equivalents of CsF and 5% of Pd(OAc)₂ under nitrogen and heat at 80° C. Monitor polymerization using UV-Vis spectroscopy and SEC chromatography. Later, add a capping agent (selected from G¹) containing appropriate functional group to the reaction mixture. Three hours later, add a second capping agent (selected from G²) to the reaction mixture. After the reaction, evaporate off the crude reaction mixture and pass it through a gel filtration column to remove small organic molecules and low MW oligomers. Later, pass the crude polymer through a Tangential flow filtration system equipped with a 100K MWCO membrane. Wash using 20% ethanol until the absorption of the filtrate diminishes.

Method 3: In a round bottom flask, dissolve both a dibromo THP monomer and a diboronic THP monomer (1:1) in a THE-water (4:1) mixture containing 10 equivalent of K₂CO₃ and 3% Pd(PPh₃)₄. Put the reaction mixture on a Schlenk line and degas with three freeze-pump-thaw cycles and then heat to 80° C. under nitrogen with vigorous stirring for 18 hours. Later, add a capping agent (selected from G¹) containing an appropriate functional group to the reaction mixture via a cannula under excess nitrogen pressure. Three hours later, add a second capping agent (selected from G²) to the reaction mixture. After the reaction, evaporate the crude reaction mixture off and pass through a gel filtration column to remove small organic molecules and low MW oligomers. Later, pass the crude polymer through a Tangential flow filtration system equipped with a 100K MWCO membrane. Wash using 20% ethanol until the absorption of the filtrate diminishes

Method 4: Alternatively, the polymerization can be accomplished by self-polymerizing a bromo-boronic ester of a THP molecule. In a round bottom flask, dissolve THP bromoboronic ester in THE-water (4:1) mixture containing 10 equivalent of K₂CO₃ and 3% Pd(PPh₃)₄. Put the reaction mixture on a Schlenk line and degas with three freeze-pump-thaw cycles and then heat to 80° C. under nitrogen with vigorous stirring for 18 hours. Later, add a capping agent (selected from G¹) containing an appropriate functional group to the reaction mixture via a cannula under excess nitrogen pressure. Three hours later, add a second capping agent (selected from G²) to the reaction mixture. After the reaction, evaporate off the crude reaction mixture and pass through a gel filtration column to remove small organic molecules and low MW oligomers. Later, pass the crude polymer through a Tangential flow filtration system equipped with a 100K MWCO membrane. Wash using 20% ethanol until the absorption of the filtrate diminishes.

### Example 2: Preparation of Water-Soluble Fluorescent THP-DHP Copolymer

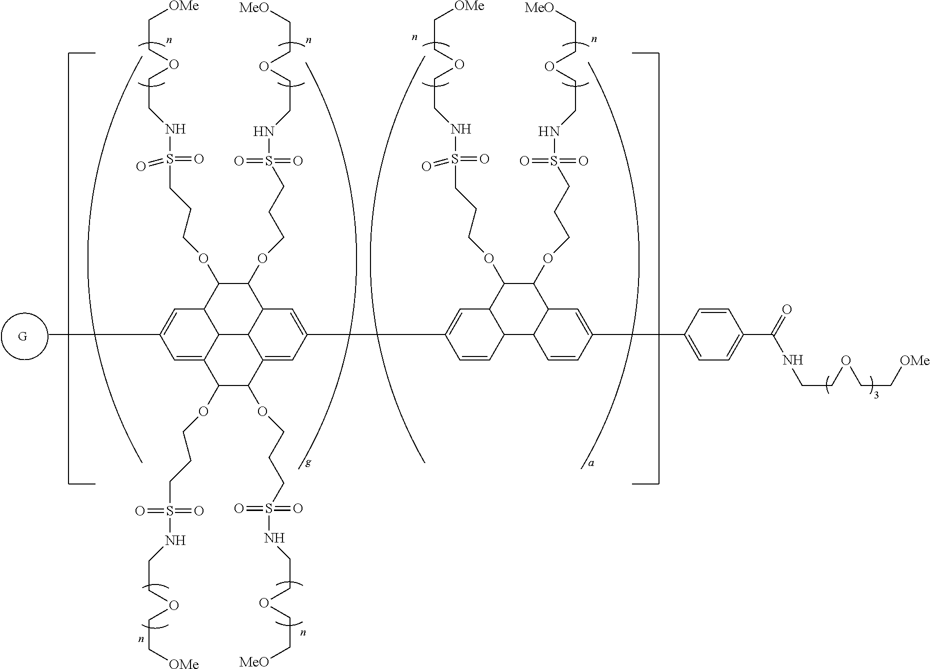

Method 1: In a round bottom flask, dissolve both a dibromo THP and a diboronic DHP monomer (1:1) is taken in a DMF-water mixture and purge with nitrogen for 10 minutes. Mix about 20 equivalents of CsF and 10% of Pd(OAc)₂ under nitrogen and heat at 80° C. Monitor polymerization using UV-Vis spectroscopy and SEC chromatography. Later add a capping agent (selected from G¹) containing an appropriate functional group to the reaction mixture. Three hours later, add a second capping agent (selected from G²) to the reaction mixture. After the reaction, evaporate off the crude reaction mixture and pass through a gel filtration column to remove small organic molecules and low MW oligomers. Later, pass the crude polymer through a Tangential flow filtration system equipped with a 100K MWCO membrane. Wash using 20% ethanol until the absorption of the filtrate diminishes.

Method 2: In a round bottom flask dissolve both a dibromo THP monomer and diboronic DHP monomer (1:1) in a THF-water (4:1) mixture containing 10 equivalents of K₂C₃ and 300 Pd(PPh₃)₄. Put the reaction mixture on a Schlenk line and degas with three freeze-pump-thaw cycles and then heat to 80° C. under nitrogen with vigorous stirring for 18 hours. Later, add a capping agent (selected from G) containing an appropriate functional group to the reaction mixture via a cannula under excess nitrogen pressure. Three hours later, add a second capping agent (selected from G²) to the reaction mixture. After the reaction, evaporate off the crude reaction mixture and pass through a gel filtration column to remove small organic molecules and low MW oligomers. Later, pass the crude polymer through a Tangential flow filtration system equipped with a 100K MWCO membrane. Wash using 20% ethanol until the absorption of the filtrate diminishes

### Example 3: Polymer Characteristics

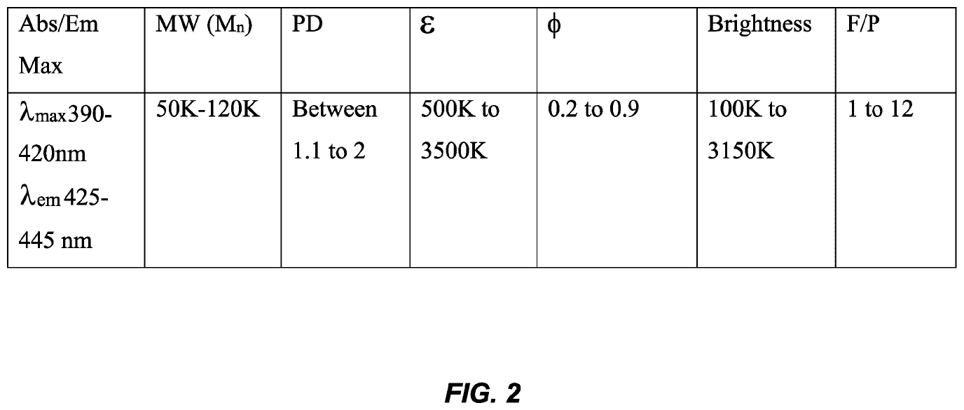

It is believed that water-soluble fluorescent polymers of the present invention possess certain physical and chemical characteristics of absorption, fluorescence, brightness, molecular weight, polydispersity, dye to protein ratio when conjugated to a binding agent (e.g., antibody etc.). In some instances, the ranges of these parameters are those shown in the table of FIG. 2.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A water-soluble fluorescent polymer comprising the structure of Formula I: 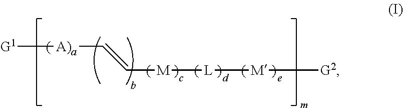
wherein:
A is aryl or heteroaryl wherein at least one A is selected from the group consisting of 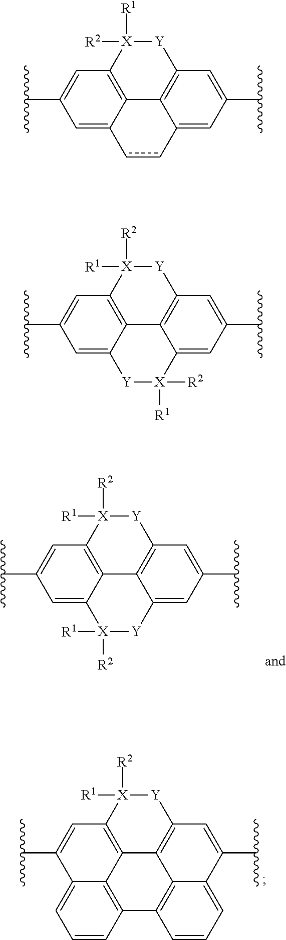
each X is independently selected from the group consisting of a C and Si;
each Y is independently selected from the group consisting of CH₂, CR¹R², and SiR¹R²;
each R¹ is independently selected from the group consisting of a water-solubilizing moiety, alkene, alkyne, cycloalkyl, haloalkyl, (hetero)aryloxy, (hetero)arylamino, PEG, carboxylic acid, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 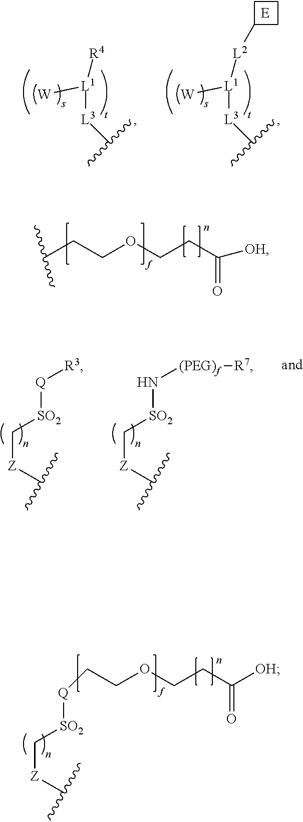
each R² is independently selected from the group consisting of a water-solubilizing moiety, a linker moiety, H, alkene, alkyne, cycloalkyl, haloalkyl, (hetero)aryloxy, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 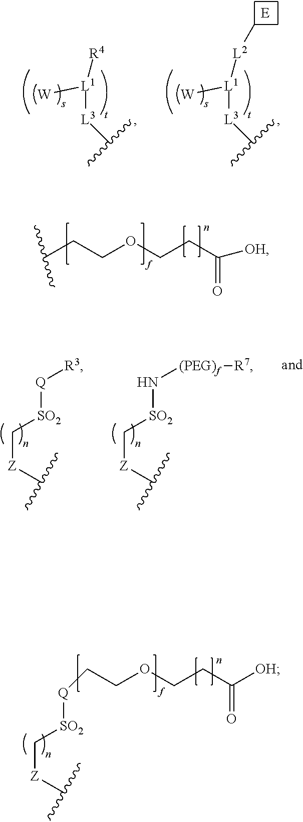
each R³ is independently a water-solubilizing moiety;
Q is a bond, NR⁴, or —CH₂;
Z is CH₂, O, or NR⁴;
W is a water solubilizing moiety;
each R⁴ is independently selected the group consisting of H, a PEG group, a water-solubilizing moiety, a linker moiety, a chromophore, a linked chromophore, a functional group, a linked functional group, a functional tag, a linked functional tag, a substrate, a linked substrate, a binding agent, a linked binding agent, L²-E, halogen, hydroxyl, alkylamino, alkyl, substituted or unsubstituted C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₅ alkyl, x′ is independently an integer from 0-20 and each y′ is independently an integer from 0-50, Z—(CH₂)ₙ—SO₂—Q—R³, a C₂-C₁₈ (hetero)aryl group, amide, amine, carbamate, carboxylic acid, carboxylate ester, maleimide, activated ester, N-hydroxysuccinimidyl, hydrazine, hydrazide, hydrazone, azide, alkyne, aldehyde, and thiol, or protected groups thereof;
each R⁷ is independently selected from the group consisting of H, hydroxyl, C₁-C₁₂alkyl, C₂-C₁₂ alkene, C₂-C₁₂ alkyne, C₃-C₁₂cycloalkyl, C₁-C₁₂haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
at least one of R¹, R², R³, or R⁴ comprises a water-solubilizing moiety;
each optional M and M′ is independently a polymer modifying unit evenly or randomly distributed along the polymer main chain and is optionally substituted with one or more optionally substituted R¹, R², R³, or R⁴ groups;
each E is independently a chromophore, acceptor dye, functional tag, substrate, or binding agent;
each optional L is a linker, optionally conjugated to a chromophore, acceptor dye, substrate, functional tag, or binding agent;
L¹, L², and L³ are each independently selected linker moieties;
G¹ and G² are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene, optionally substituted fluorene, optionally substituted dihydrophenanthrene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, optionally conjugated to a substrate, chromophore, functional tag, or a binding agent;
a, c, d, and e define the mol % of each unit within the structure which each can be evenly or randomly repeated and where each a is a mol % from 10 to 100%, each c is a mol % from 0 to 90%, each d is a mol % from 0 to 25%, and each e is a mol % from 0 to 90%;
each b is independently 0 or 1;
each f is independently an integer from 0 to 50;
m is an integer from 1 to about 10,000;
each n is independently an integer from 1 to 20;
each s is independently 1 or 2; and
each t is independently 1, 2, or 3.

2. The water-soluble fluorescent polymer of claim 1, wherein the polymer has the structure of at least one member selected from the group consisting of Formula (Ia), (Ib), (Ic), (Id), (IIa), (IIb), (IIc), (IId), (IIIa), (IIIb), (IIIc), (IIId), (IVa), (IVb), (IVc), and (IVd):

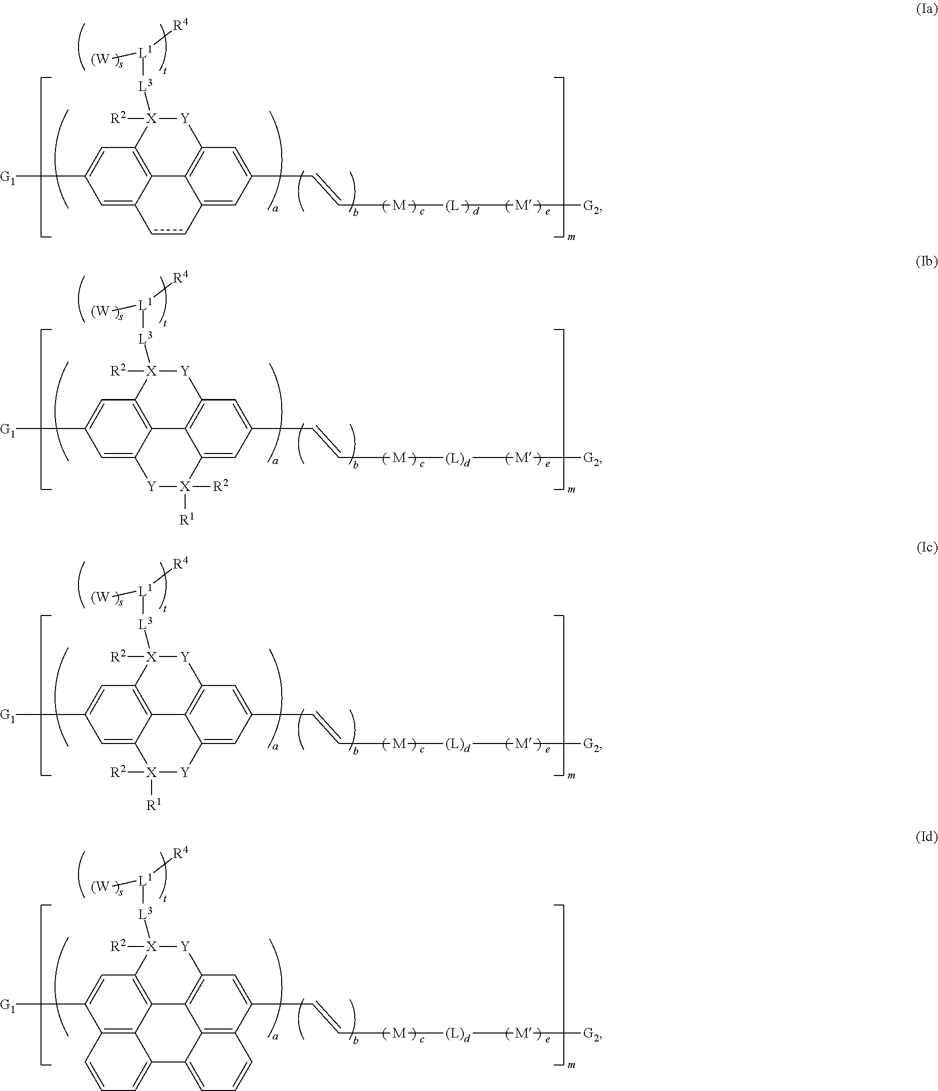

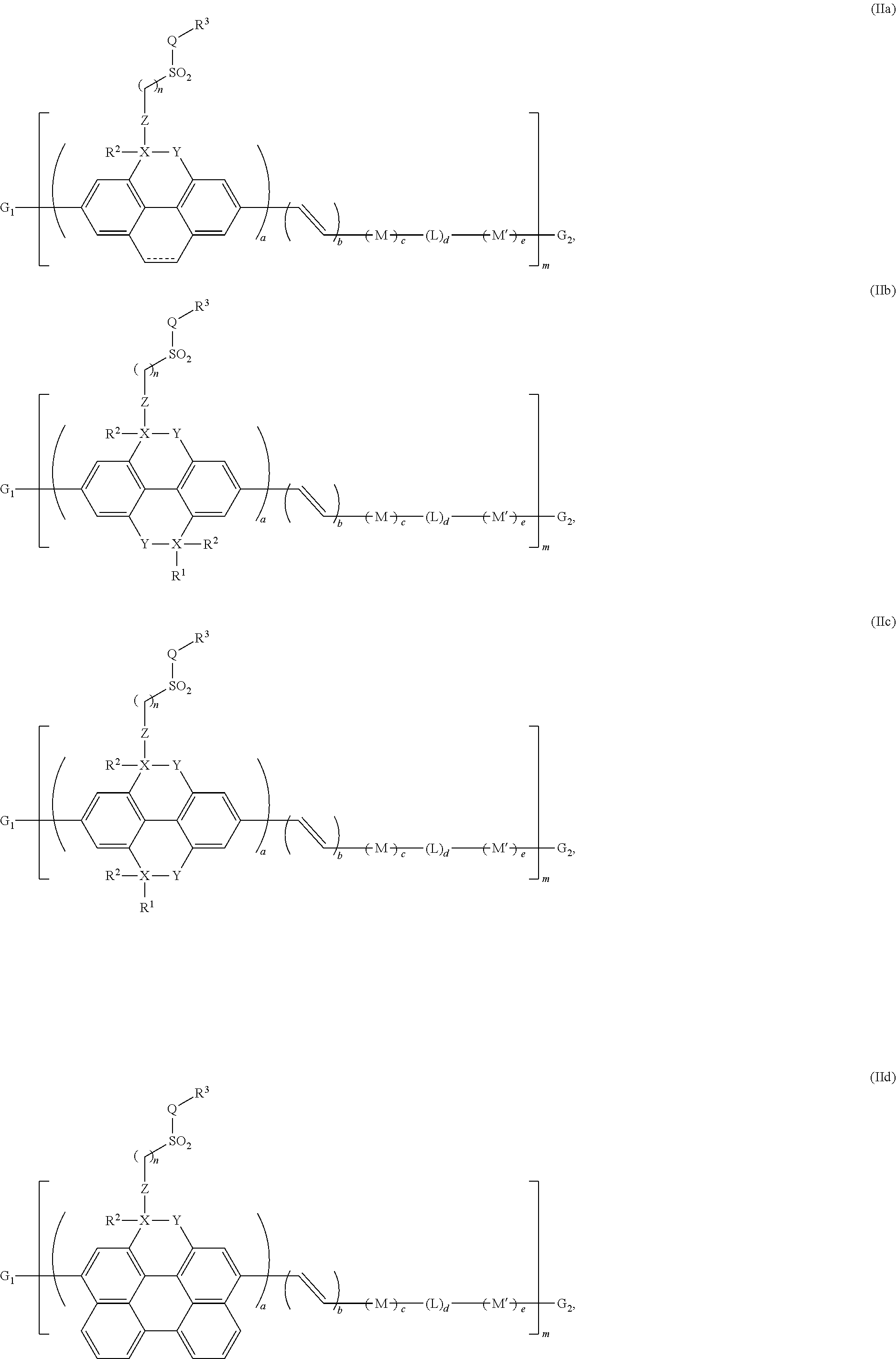

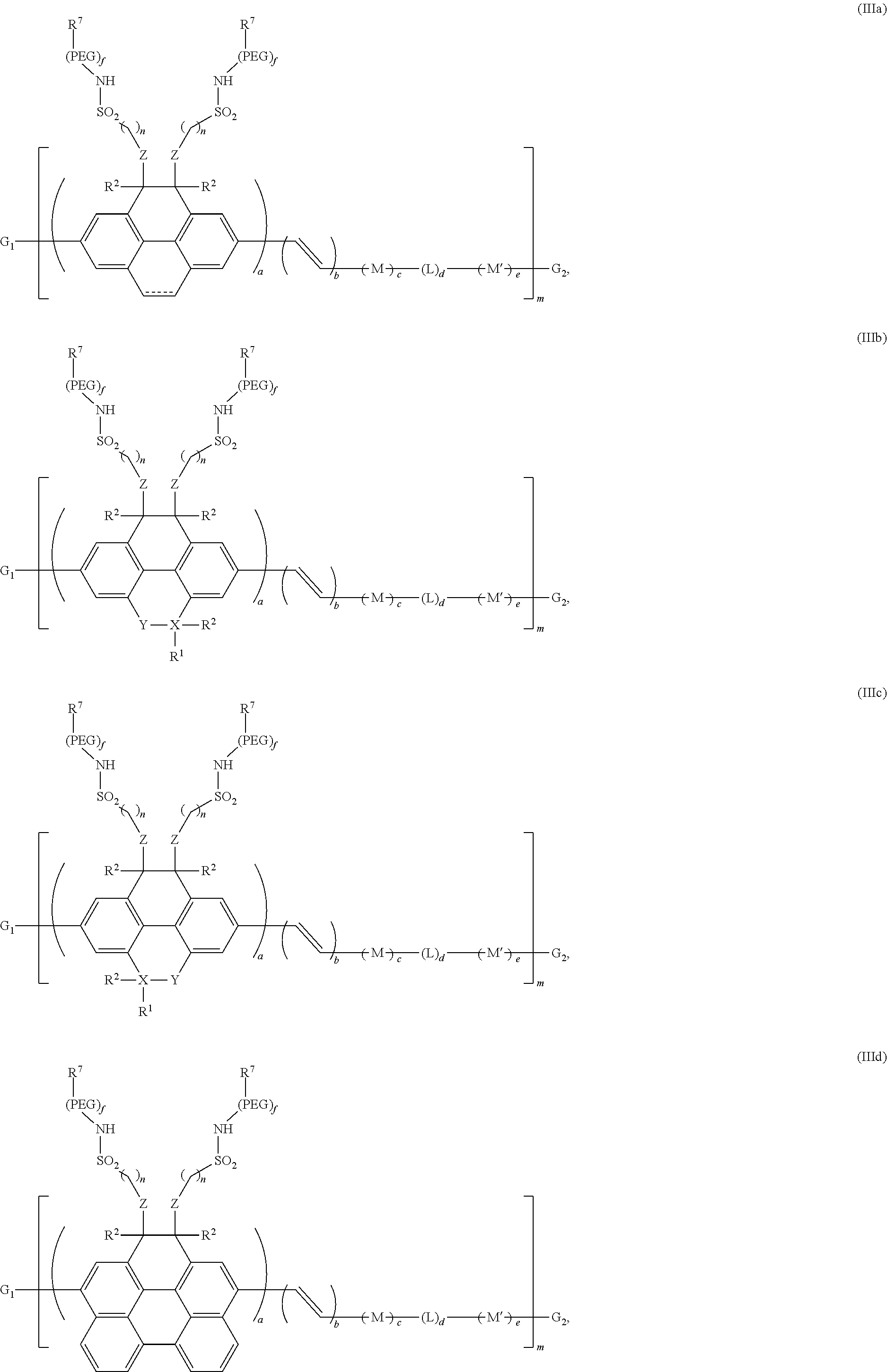

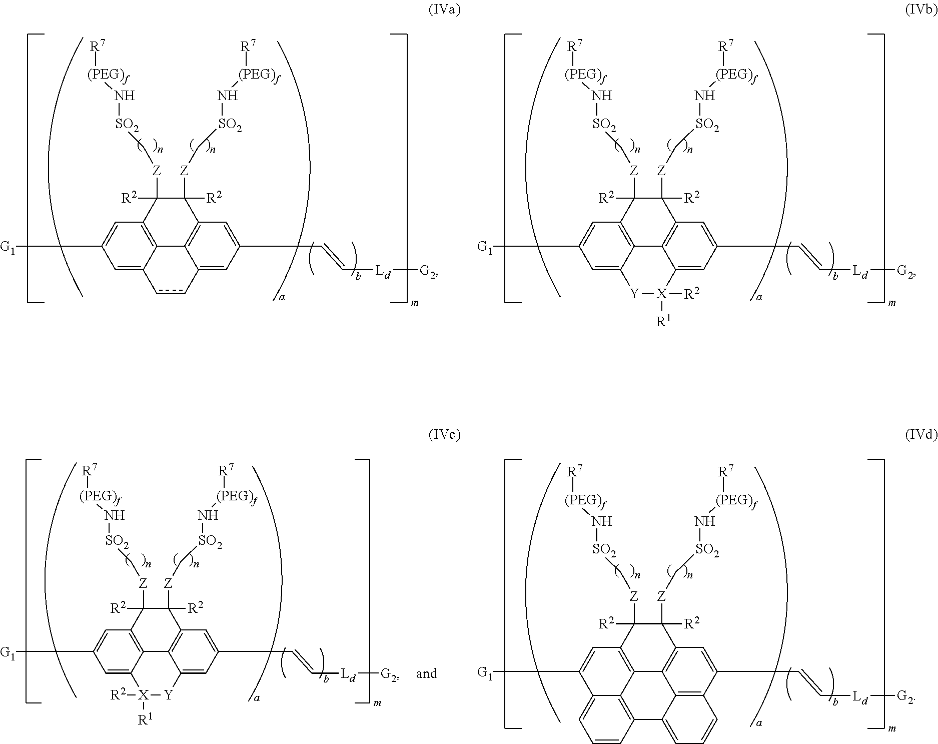

3. The water-soluble fluorescent polymer of claim 1, wherein the polymer has the structure of at least one member selected from the group consisting of Formula (Va)-(Vd):

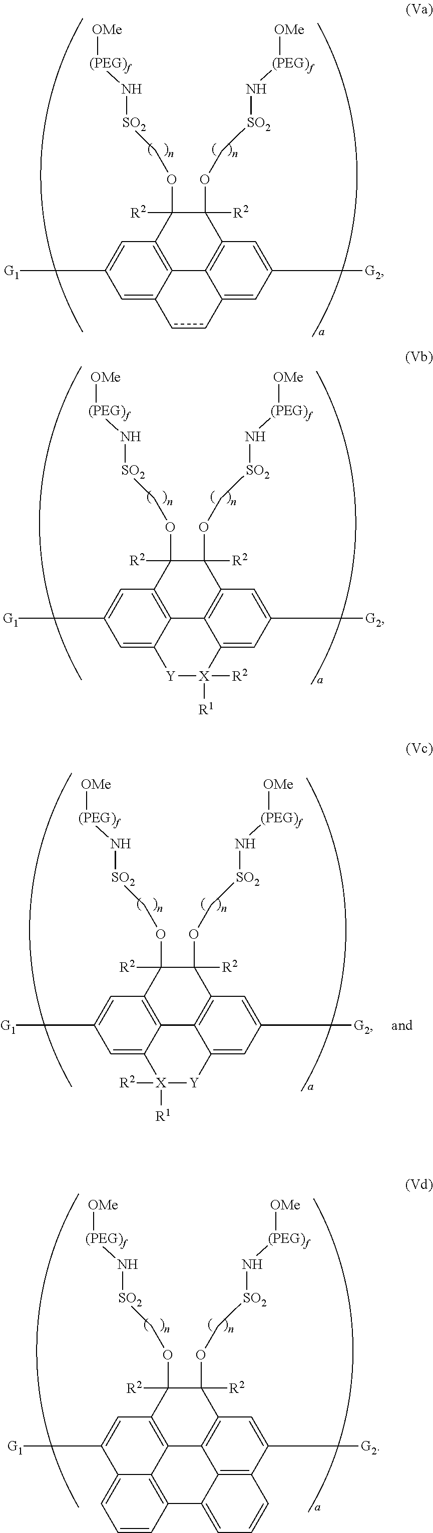

4. The water-soluble fluorescent polymer of claim 1, wherein the polymer has the structure of at least one member selected from the group consisting of Formula (VIa), (VIb), (VIIa), (VIIb), (VIIIa), and (VIIIb):

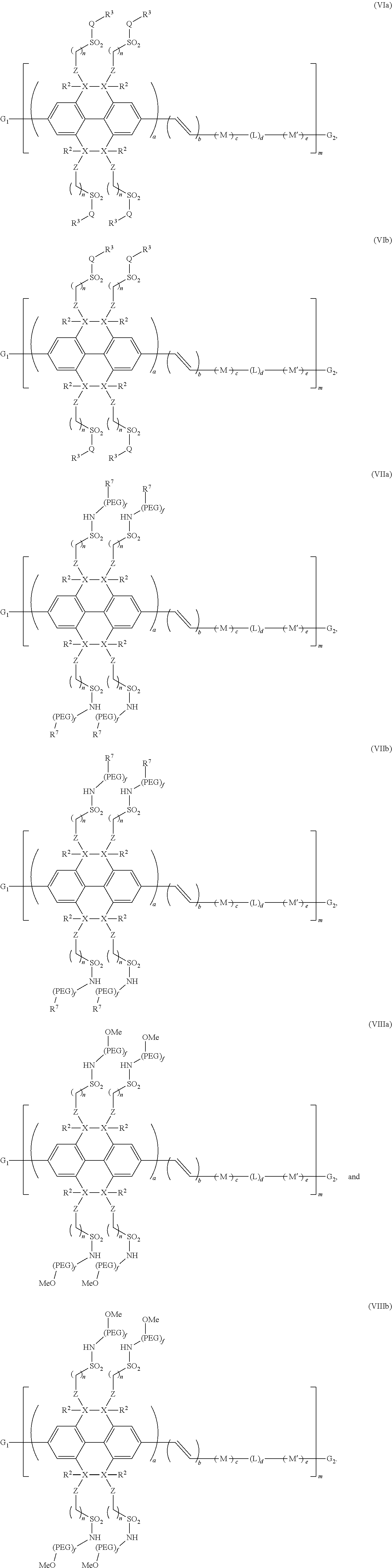

5. The water-soluble fluorescent polymer of claim 1, wherein the polymer is a water-soluble fluorescent copolymer and has the structure of any one of Formulae (IX), (X), (XI), (XII), (XIII), (XIV), (XV), (XVI), and (XVII):

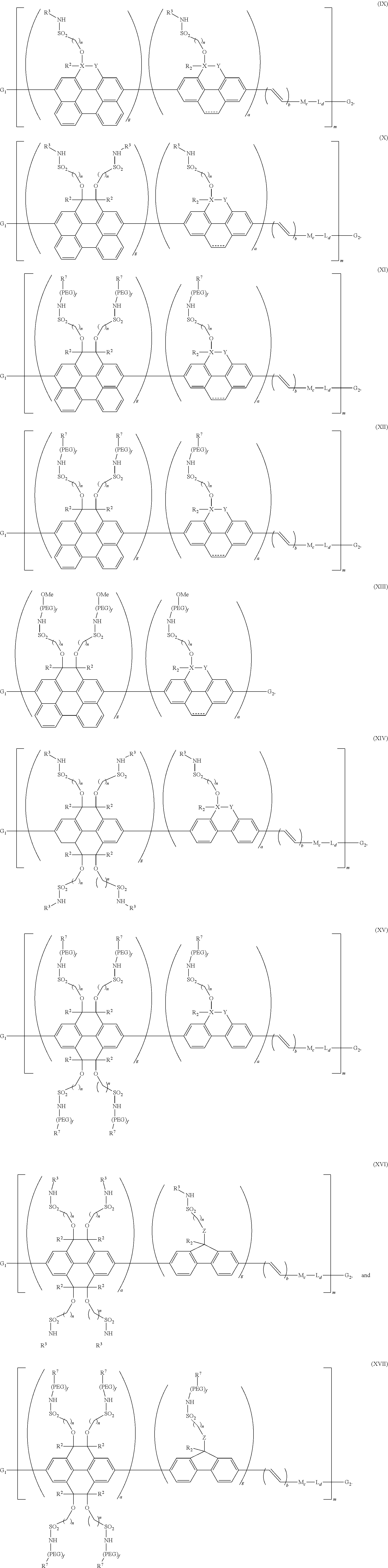

6. The water-soluble fluorescent polymer of claim 1, wherein each M and M′ is independently an optionally substituted arylene or optionally substituted heteroarylene.

7. The water-soluble fluorescent polymer of claim 6, wherein each M and M′ is independently selected from the group consisting of: substituted or unsubstituted 1,4-phenyl, substituted or unsubstituted 1,3-phenyl, substituted or unsubstituted 4,4′-biphenyl, substituted or unsubstituted 2,5-pyridyl, substituted or unsubstituted 2,6-pyridyl, substituted or unsubstituted 9,10-dihydrophenanthrene, substituted or unsubstituted fluorene, substituted or unsubstituted binaphthyl, 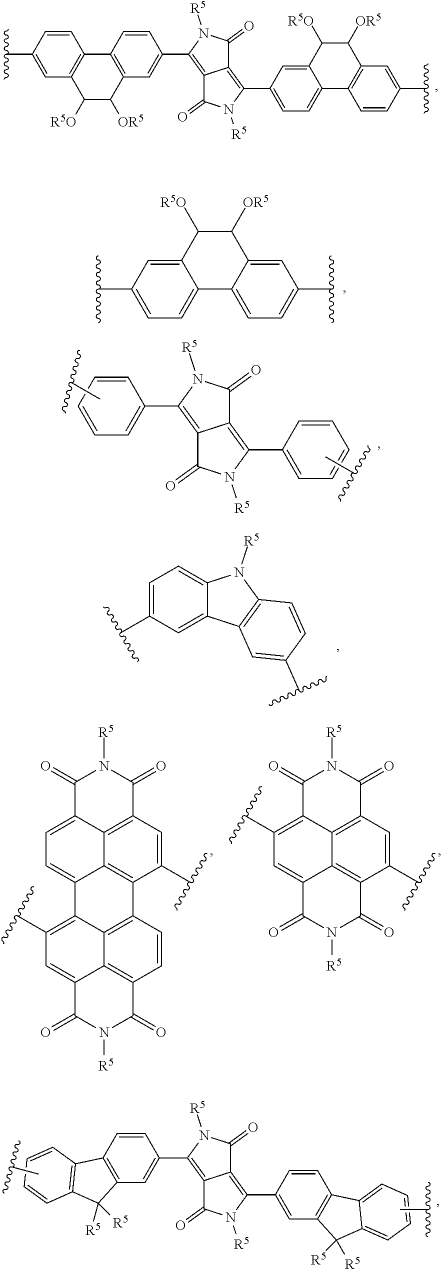 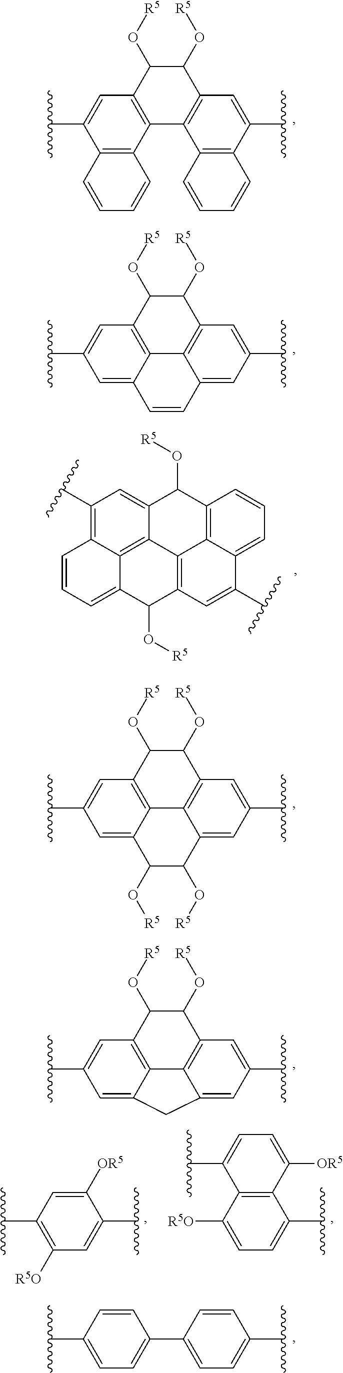 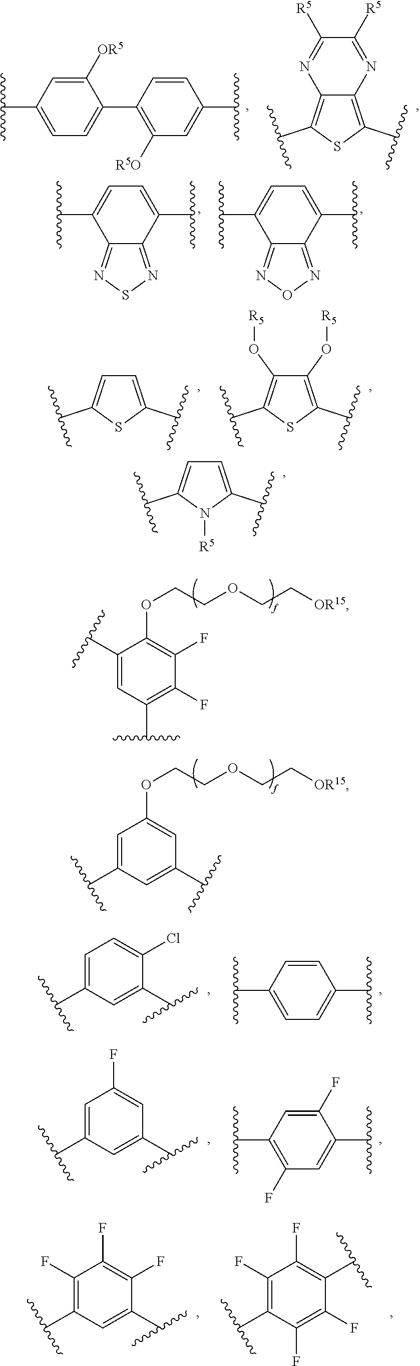 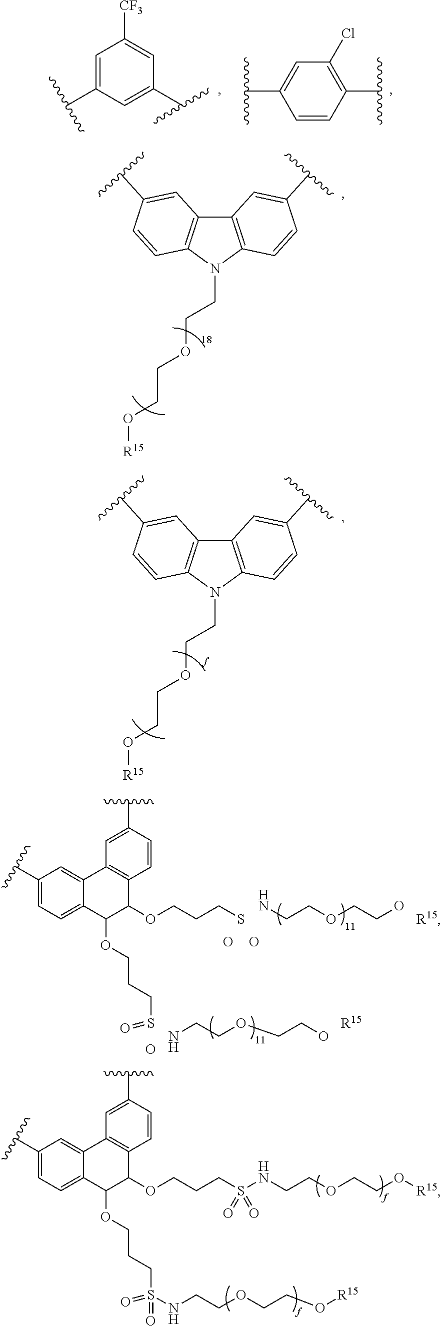 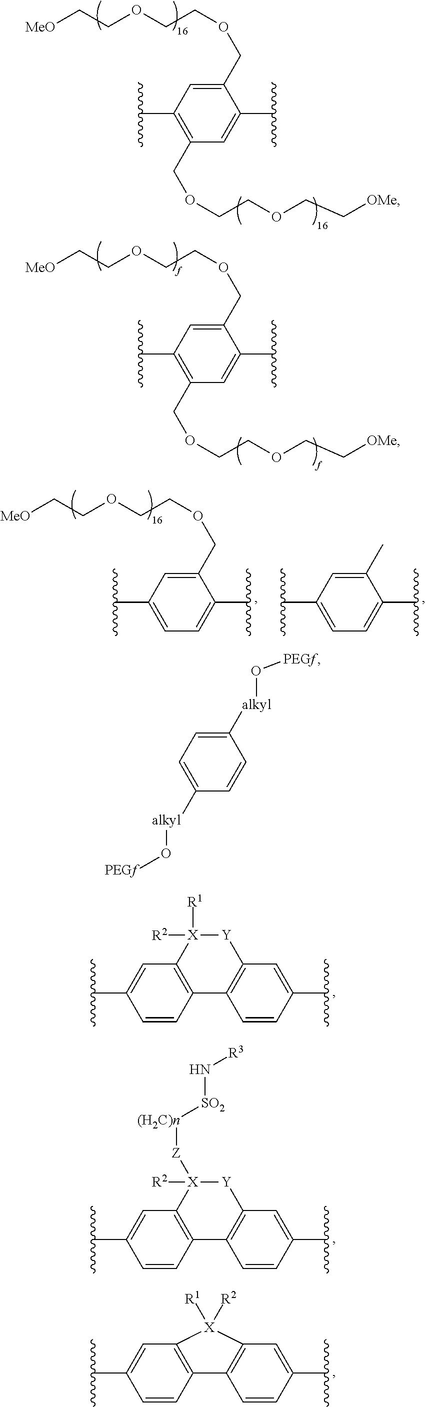 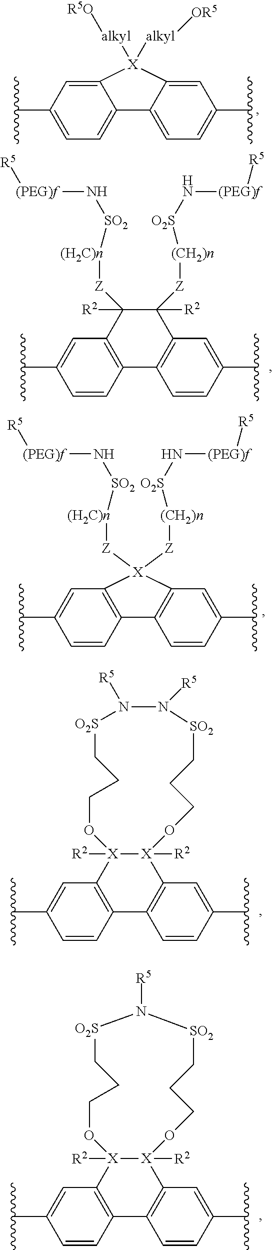 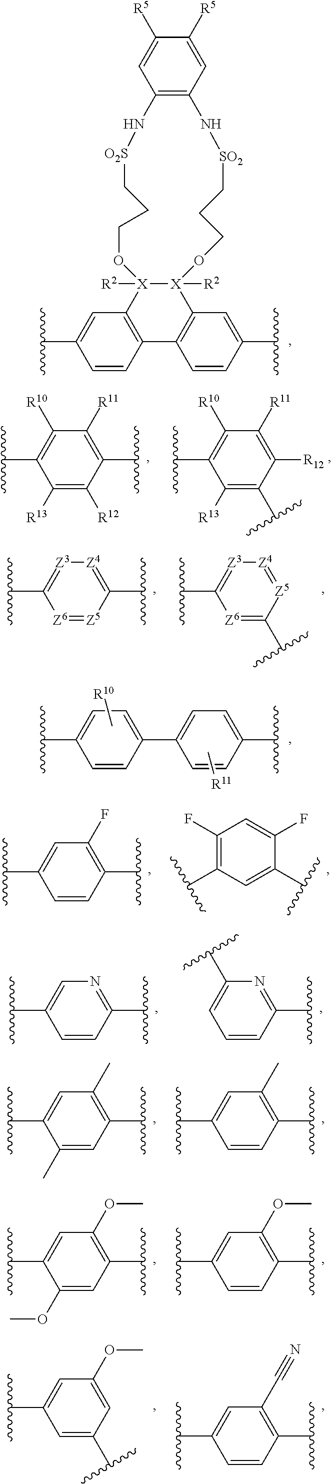 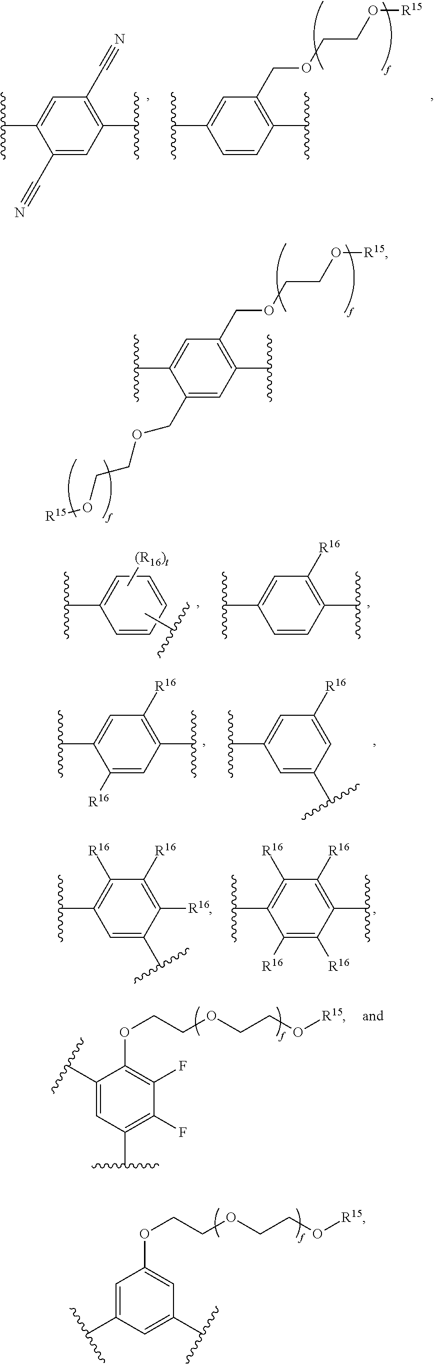
wherein each M and M′ is optionally substituted, and optionally terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, amide, sulfonamide, ether, thioether, thiocarbamate, hydroxyl, iodoacetyl, hydrazido, hydrazino, ketone, phosphine, epoxide, urea, thiourea, thioester, imine, disulfides, and protected groups thereof, optionally conjugated to a substrate, chromophore, acceptor dye, functional tag, or binding agent, and
each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, a C₂-C₁₅ (hetero)aryl group, C₂-C₁₅ (hetero)aryloxy, C₂-C₁₅ (hetero)arylamino, carboxylic acid, carboxylate ester, (CH₂)_{x′}(OCH₂—CH₂)_{y′}OR¹⁵, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50;
each Z³-Z⁶ is independently CR¹⁴ or N, wherein at least one Z³-Z⁶ is N;
each R¹⁰-R¹⁴ is independently selected from the group consisting of hydrogen, halogen, cyano, alkoxy, substituted alkoxy, alkyl, substituted alkyl, alkene, substituted alkene, alkyne, azide, PEG moiety, substituted PEG moiety, and water solubilizing moiety;
each R¹⁵ is independently H or lower alkyl;
each R¹⁶ is independently selected from the group consisting of H, F, Cl, —CF₃, and —(CH₂CH₂)OR¹⁵.

8. The water-soluble fluorescent polymer of claim 1, wherein each L is independently selected from the group consisting of 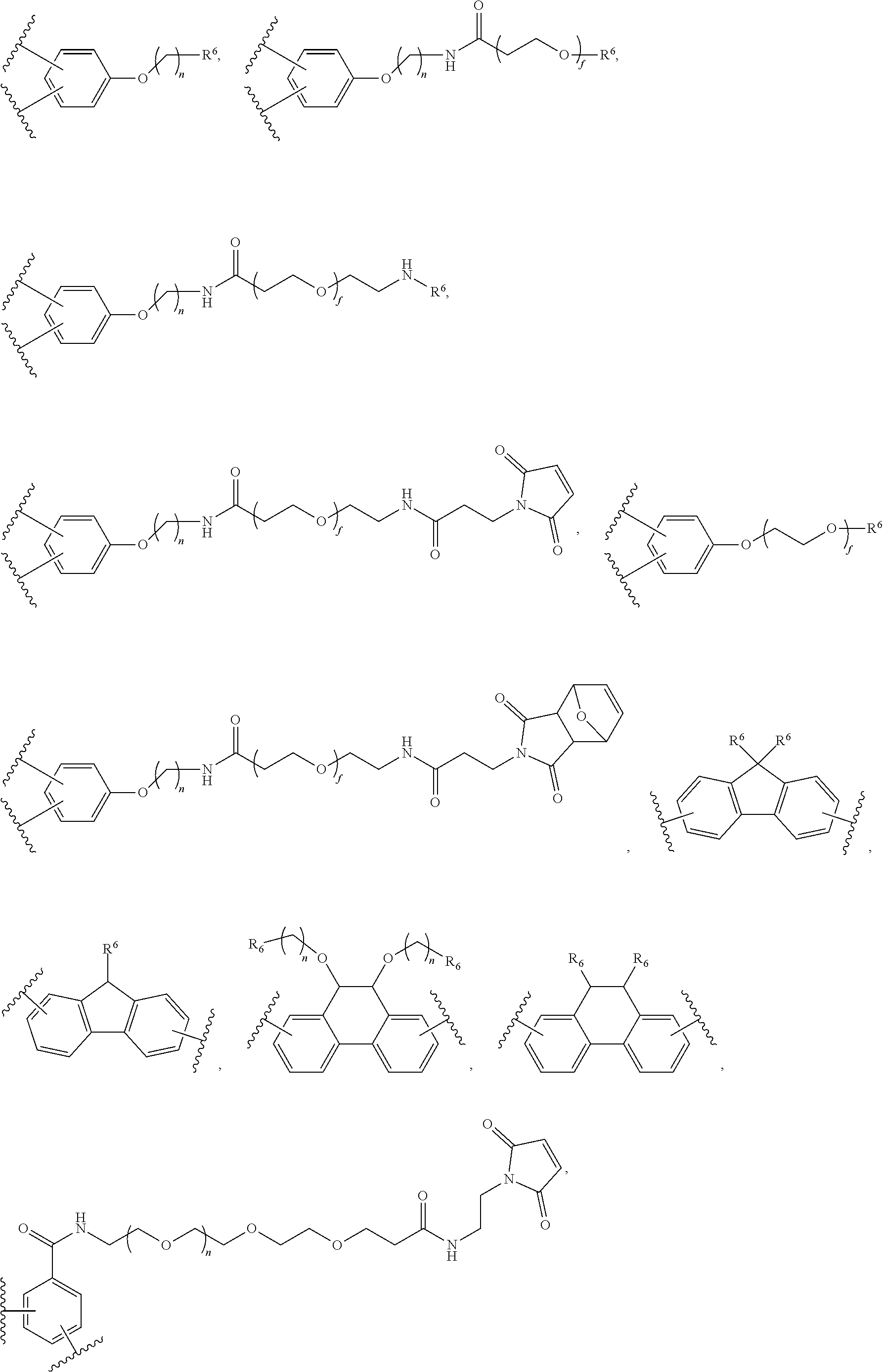 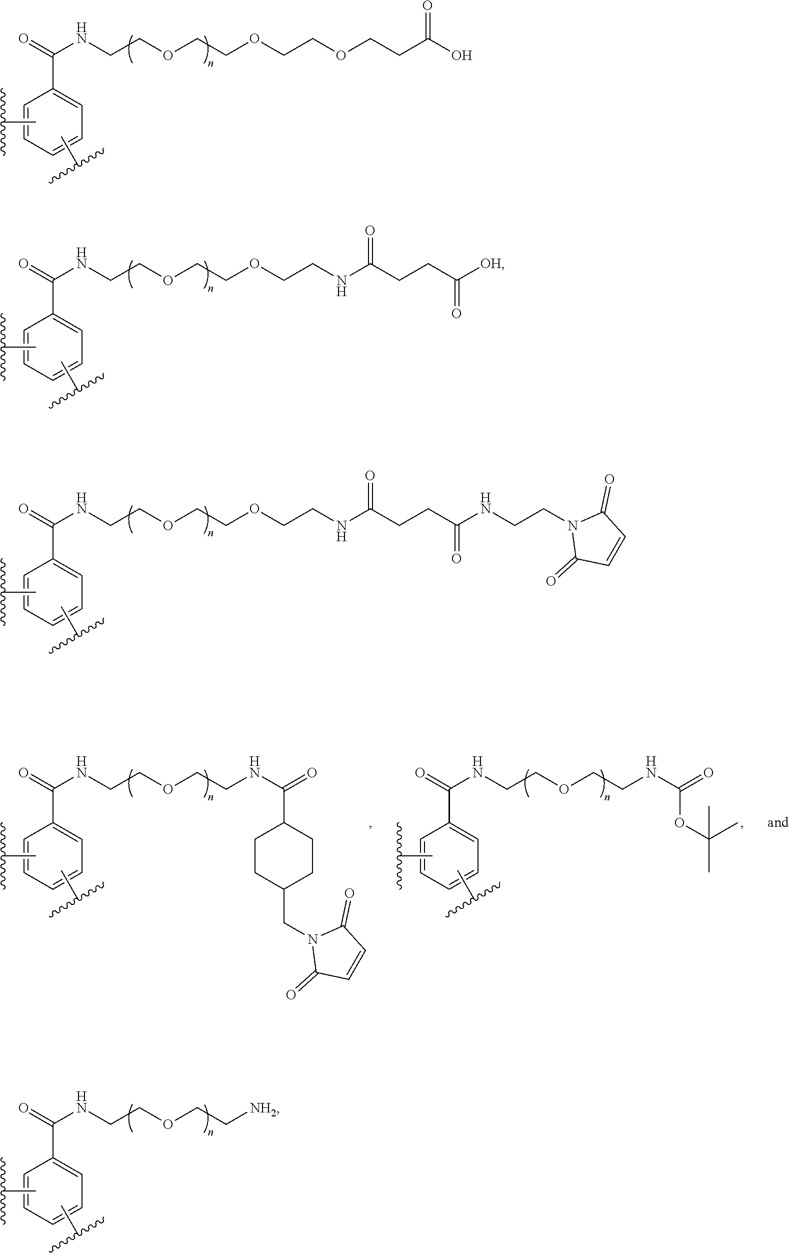
wherein
each R⁶ is independently selected from the group consisting of H, OH, SH, NHCOO-t-butyl, (CH₂)ₙCOOH, (CH₂)ₙCOOR¹⁵, (CH₂)ₙCOOCH₃, (CH₂)ₙNH₂, (CH₂)ₙNH—(CH₂)ᵣ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}OR¹⁵, (CH₂)ₙNHCO(CH₂CH₂O)_{f}OCH₃, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OR¹⁵, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino, azide, benzyl optionally substituted with one or more halogen, hydroxyl, C₁-C₁₂ alkoxy, or (OCH₂CH₂)_{f}OCH₃, 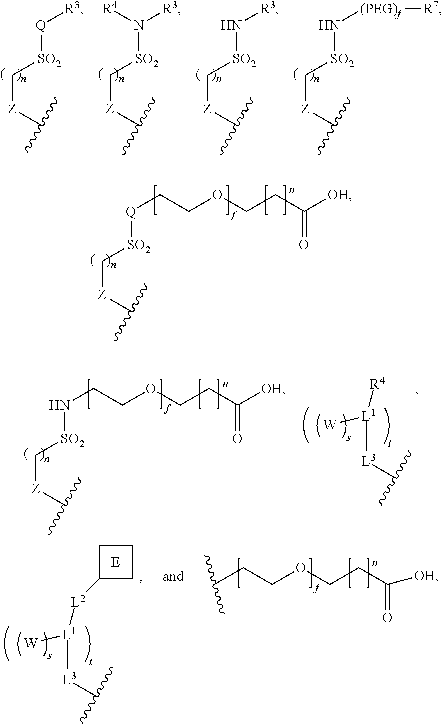
optionally conjugated to a substrate, chromophore, acceptor dye, functional tag, or binding agent.

9. The water-soluble fluorescent polymer of claim 1, wherein G¹ and G² are each independently selected from the group consisting of:
a. optionally substituted dihydrophenanthrene (DHP), tetrahydropyrene (THP), optionally substituted fluorene, aryl substituted with one or more pendant chains terminated with a functional group, and a heteroaryl substituted with one or more pendant chains terminated with a functional group; and b. 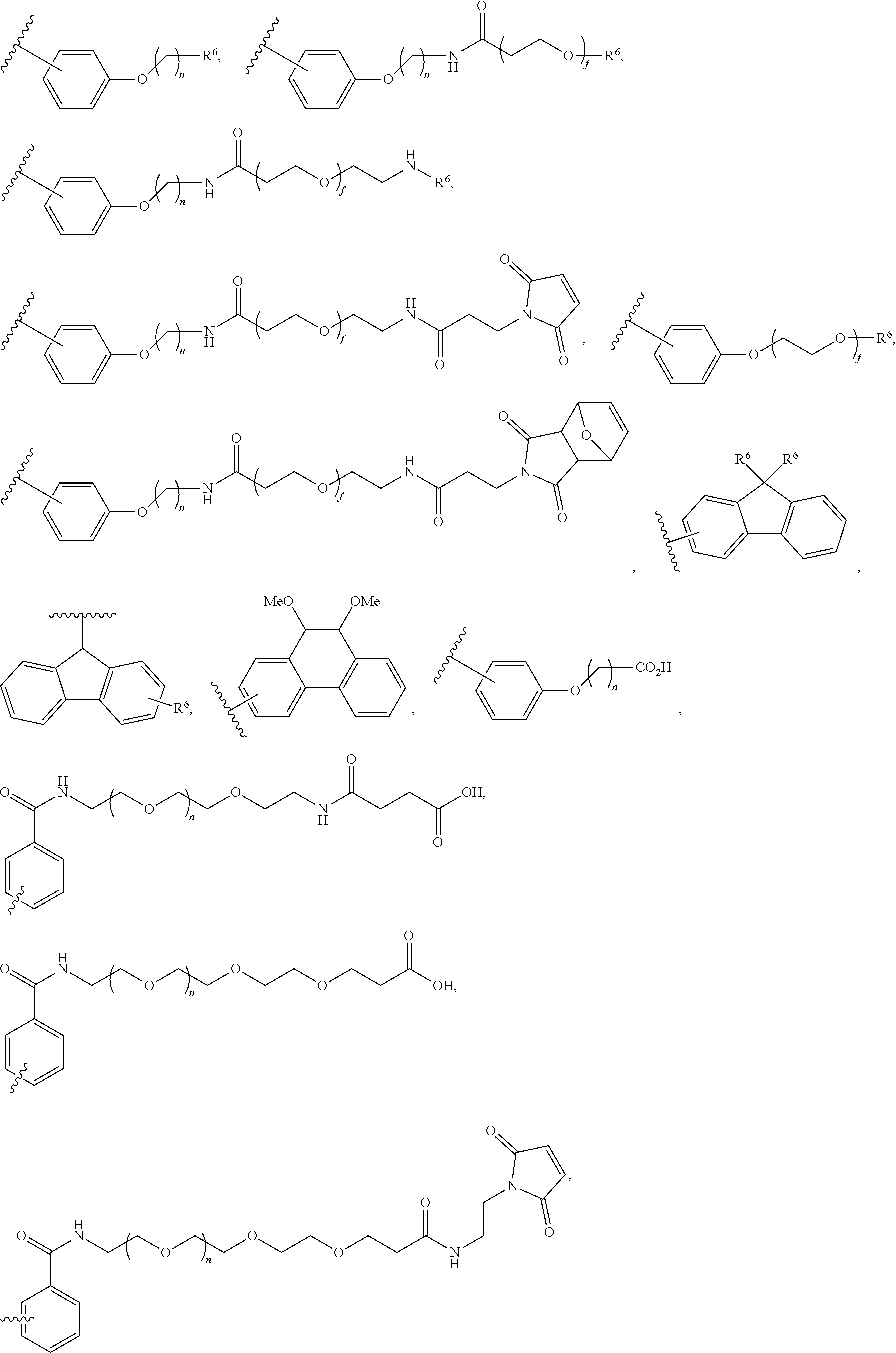 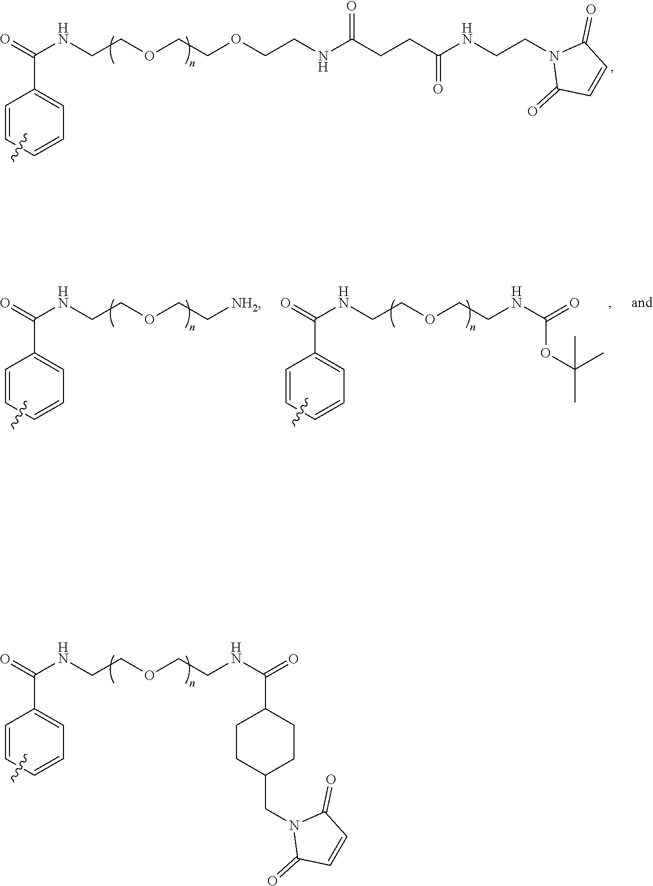
wherein,
each R⁶ is independently selected from the group consisting of H, OH, SH, NHCOO-t-butyl, (CH₂)ₙCOOH, (CH₂)ₙCOOR¹⁵, (CH₂)ₙCOOCH₃, (CH₂)ₙ(CH₂CH₂O)_{f}COOH, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}OR¹⁵, (CH₂)ₙNHCO(CH₂CH₂O)_{f}OCH₃, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OR¹⁵,(CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂halo alkyl, C₁-C₁₂ (hetero)aryl, C₁-C₁₂ (hetero)arylamino, azide, and benzyl optionally substituted with one or more halogen, hydroxyl, C₁-C₁₂ alkoxy, or (OCH₂CH₂)_{f}OCH₃, 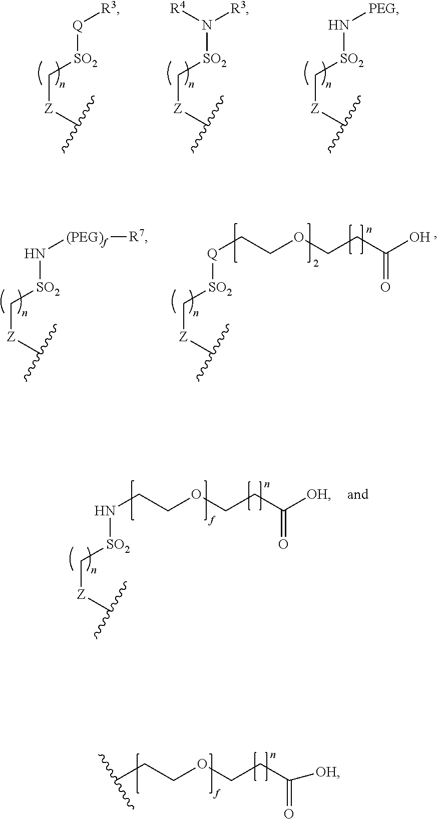
and
each of G¹ and/or G² is optionally conjugated to a substrate, chromophore, acceptor dye, functional tag, or binding agent.

10. The water-soluble fluorescent polymer of claim 1, further comprising a binding agent linked to said polymer, optionally wherein the binding agent is selected from the group consisting of an antibody, antibody fragment, protein, peptide, affinity ligand, carbohydrate, lipid, nucleic acid, and an aptamer.

11. The water-soluble fluorescent polymer of claim 1, further comprising an acceptor dye or chromophore linked to said polymer, optionally wherein the acceptor dye is linked to monomer A or linker L of said polymer.

12. A tandem polymer dye comprising
the water-soluble fluorescent polymer of claim 1; and an acceptor dye or chromophore linked to said polymer, optionally further comprising a binding agent linked to said polymer, optionally wherein the binding agent is selected from the group consisting of an antibody, antibody fragment, protein, peptide, affinity ligand, carbohydrate, lipid, nucleic acid, and an aptamer.

13. A method for detecting an analyte in a sample comprising:
providing a sample that is suspected of containing the analyte; and
contacting the sample with a binding agent conjugated to a water-soluble fluorescent polymer as defined in claim 1, wherein the binding agent is capable of interacting with the analyte.

14. The method of claim 13, wherein the binding agent is an antibody, antibody fragment, protein, peptide, affinity ligand, carbohydrate, lipid, nucleic acid or an aptamer.

15. The method of claim 14, wherein the binding agent is an antibody, optionally wherein:
a. the method is configured for flow cytometry;
b. the water-soluble fluorescent polymer is bound to a substrate;
c. the analyte is a protein expressed on a cell surface;
d. the method is configured as an immunoassay; or
e. the method further comprises providing additional binding agents for detecting additional analytes simultaneously.

16. A kit comprising a water soluble fluorescent polymer according to claim 1.
